(19) United States
(12) Patent Application Publication
(10) Pub. No.: US 2024/0228554 A9
(43) Pub. Date: Jul. 11, 2024

(54) PROTEINS AND NUCLEIC ACIDS USEFUL IN VACCINES TARGETING STAPHYLOCOCCUS AUREUS

(71) Applicant: Evaxion Biotech A/S, Hørsholm (DK)

(72) Inventors: Niels Iversen Møller, Hørsholm (DK); Andreas Holm Mattson, Hørsholm (DK)

(21) Appl. No.: 18/479,501

(22) Filed: Oct. 2, 2023

Related U.S. Application Data
(63) Continuation of application No. 16/600,031, filed on Oct. 11, 2019, now Pat. No. 11,807,669.
Application No. 16/600,031 is a division of application No. 15/101,791, filed on Jun. 3, 2016, now abandoned.

(51) Int. Cl. C07K 14/31; A61K 39/085; C07K 16/12; C12Q 1/689; G01N 33/569

(52) U.S. Cl. CPC C07K 14/31; A61K 39/085; C07K 16/1271; C12Q 1/689; G01N 33/56938; A61K 2039/55505; G01N 2333/31; G01N 2500/04

(57) ABSTRACT

The present invention relates to proteins and nucleic acids derived from Staphylococcus aureus as well as therapeutic and diagnostic uses of the proteins and nucleic acids.

### CROSS REFERENCE TO RELATED APPLICATIONS

In accordance with 37 C.F.R. 1.76, a claim of priority is included in an Application Data Sheet filed concurrently herewith. Accordingly, the present invention claims priority as a continuation of U.S. Utility application Ser. No. 16/600,031, entitled “PROTEINS AND NUCLEIC ACIDS USEFUL IN VACCINES TARGETING STAPHYLOCOCCUS AUREUS”, filed Oct. 11, 2019, which is a divisional of U.S. patent application Ser. No. 15/101,791, entitled “PROTEINS AND NUCLEIC ACIDS USEFUL IN VACCINES TARGETING STAPHYLOCOCCUS AUREUS”, filed Jun. 3, 2016, which is a § 371 national stage entry of International Application No. PCT/EP2014/076398, entitled “PROTEINS AND NUCLEIC ACIDS USEFUL IN VACCINES TARGETING STAPHYLOCOCCUS AUREUS”, filed Dec. 3, 2014, which claims the benefit of the priority of European Patent Application No. 13195472.9, entitled “PROTEINS AND NUCLEIC ACIDS USEFUL IN VACCINES TARGETING STAPHYLOCOCCUS AUREUS”, filed Dec. 12, 2013, the entire contents of each are incorporated herein by reference.

### REFERENCE TO AN ELECTRONIC SEQUENCE LISTING

The contents of the electronic sequence listing (4564167CONSequencelisting.xml; Size: 99 KB and Date of Creation: Jan. 8, 2024) is herein incorporated by reference in its entirety.

### FIELD OF THE INVENTION

The present invention relates to the field of antimicrobial prophylaxis and therapy. In particular the present invention relates to novel proteins and polynucleotides derived from Staphylococcus aureus. The invention further relates to vectors comprising the polynucleotides, transformed host organisms expressing the polynucleotides, antibodies (mono- or polyclonal) specific for the polypeptides as well as diagnostic, prophylactic and therapeutic uses and methods. Finally, also methods of preparation are part of the invention.

### BACKGROUND OF THE INVENTION

Bacterial infections are in most instances successfully treated by administration of antibiotics to patients in need thereof. However, due to careless or thoughtless use of powerful antibiotics, many pathological germs become resistant against antibiotics over time. One threatening example is Staphyloccocus aureus. In particular in hospitals this bacterium is of relevance. So-called Methicillin Resistant S. aureus (MRSA) strains jeopardize patient's survival in hospitals, in particular after surgery.

Vaccination is considered to be a very effective method of preventing infectious diseases in human and veterinary health care. Vaccination is the administration of immungenically effective amounts of antigenic material (the vaccine) to produce immunity to a disease/disease-causing pathogenic agent. Vaccines have contributed to the eradication of smallpox, the near eradication of polio, and the control of a variety of diseases, including rubella, measles, mumps, chickenpox, typhoid fever.

Before “the genomic era”, vaccines were based on killed or live attenuated, microorganisms, or parts purified from them. Subunit vaccines are considered as a modern upgrade of these types of vaccine, as the subunit vaccines contain one or more protective antigens, which are more or less the weak spot of the pathogen. Hence, in order to develop subunit vaccines, it is critical to identify the proteins, which are important for inducing protection and to eliminate others.

An antigen is said to be protective if it is able to induce protection from subsequent challenge by a disease-causing infectious agent in an appropriate animal model following immunization.

The empirical approach to subunit vaccine development, which includes several steps, begins with pathogen cultivation, followed by purification into components, and then testing of antigens for protection. Apart from being time and labour consuming, this approach has several limitations that can lead to failure. It is not possible to develop vaccines using this approach for microorganisms, which cannot easily be cultured and only allows for the identification of the antigens, which can be obtained in sufficient quantities. The empirical approach has a tendency to focus on the most abundant proteins, which in some cases are not immuno-protective. In other cases, the antigen expressed during in vivo infection is not expressed during in vitro cultivation. Furthermore, antigen discovery by use of the empirical approach demands an extreme amount of proteins in order to discover the protective antigens, which are like finding needles in the haystack. This renders it a very expensive approach, and it limits the vaccine development around diseases, which is caused by pathogens with a large genome or disease areas, which perform badly in a cost-effective perspective.

### OBJECT OF THE INVENTION

It is an object of embodiments of the invention to provide S. aureus derived antigenic polypeptides that may serve as constituents in vaccines against S. aureus infections and in diagnosis of S. aureus infections. It is also an object to provide nucleic acids, vectors, transformed cells, vaccine compositions, and other useful means for molecular cloning as well as for therapy and diagnosis with relevance for S. aureus.

### SUMMARY OF THE INVENTION

It has been found by the present inventor(s) that S. aureus, in particular drug resistant S. aureus, expresses a number of hitherto unknown putatively surface exposed proteins which are candidates as vaccine targets as well as candidates as immunizing agents for preparation of antibodies that target S. aureus.

So, in a first aspect the present invention relates to a polypeptide comprising
a) an amino acid sequence selected from the group consisting of any one of SEQ ID NOs: 1-16 and 49, or
b) an amino acid sequence consisting of at least or exactly or at most 5 contiguous amino acid residues from any one of SEQ ID NOs: 1-16 and 49, or
c) an amino acid sequence having a sequence identity of at least 60% with the amino acid sequence of a),
d) an amino acid sequence having a sequence identity of at least 60% with the amino acid sequence of b), or
e) an assembly of amino acids derived from any one of SEQ ID NOs: 1-16 and 49, which has essentially the same 3D conformation as in the protein from which said assembly is derived so as to constitute a B-cell epitope, said polypeptide being antigenic in a mammal.

In another aspect, the invention relates to an isolated nucleic acid fragment, which comprises
i) a nucleotide sequence encoding a polypeptide of the invention, or
ii) a nucleotide sequence consisting of any one of SEQ ID NOs: 17-48, 50, and 51.
iii) a nucleotide sequence consisting of at least or exactly or at most 10 consecutive nucleotides in any one of SEQ ID NOs: 17-48, 50, and 51,
iv) a nucleotide sequence having a sequence identity of at least 60% with the nucleotide sequence in i) or ii),
v) a nucleotide sequence having a sequence identity of at least 60% with the nucleotide sequence in iii),
vi) a nucleotide sequence complementary to the nucleotide sequence in i)-v), or
vii) a nucleotide sequence which hybridizes under stringent conditions with the nucleotide sequence in i)-vi).

In a third aspect, the invention relates to a vector comprising the nucleic acid of the invention, such as a cloning vector or an expression vector.

In fourth aspect, the invention relates to a cell which is transformed so as to carry the vector of the invention.

In a fifth aspect, the invention relates to a pharmaceutical composition comprising a polypeptide of the invention, a nucleic acid fragment of the invention, a vector of the invention, or a transformed cell of the invention, and a pharmaceutically acceptable carrier, vehicle or diluent.

In a sixth aspect, the invention relates to a method for inducing immunity in an animal by administering at least once an immunogenically effective amount of a polypeptide of the invention, a nucleic acid fragment of the invention, a vector of the invention, a transformed cell of the invention, or a pharmaceutical composition of the fifth aspect of the invention so as to induce adaptive immunity against S. aureus in the animal.

In a seventh and eighth aspect, the invention relates to 1) a polyclonal antibody in which the antibodies specifically bind to at least one polypeptide of the invention, and which is essentially free from antibodies binding specifically to other S. aureus polypeptides, and to 2) an isolated monoclonal antibody or antibody analogue which binds specifically to a polypeptide of the invention. In a related ninth aspect, the invention relates to a pharmaceutical composition comprising such a polyclonal or monoclonal antibody and a pharmaceutically acceptable carrier, vehicle or diluent.

In a 10^{th} aspect, the invention relates to a method for prophylaxis, treatment or amelioration of infection with S. aureus, comprising administering a therapeutically effective amount of an antibody of the 7^{th} or 8^{th} aspect of the invention or a pharmaceutical composition of the eighth aspect to an individual in need thereof.

In an 11^{th} aspect, the invention relates to a method for determining, quantitatively or qualitatively, the presence of S. aureus, in particular the presence of multi-resistant S. aureus, in a sample, the method comprising contacting the sample with an antibody of aspects 8 or 9 of the invention and detecting the presence of antibody bound to material in the sample.

In an 12^{th} aspect of the invention is provided a method for determining, quantitatively or qualitatively, the presence of antibodies specific for S. aureus, in particular the presence of antibodies specific for multi-resistant S. aureus, in a sample, the method comprising contacting the sample with a polypeptide of the invention and detecting the presence of antibody that specifically bind said polypeptide.

In a 13^{th} aspect, the invention relates to a method for determining, quantitatively or qualitatively, the presence of a nucleic acid characteristic of S. aureus, in particular the presence of a nucleic acid characteristic of multi-resistant S. aureus, in a sample, the method comprising contacting the sample with a nucleic acid fragment of the invention and detecting the presence of nucleic acid in the sample that hybridizes to said nucleic acid fragment.

In a 14^{th} aspect, the invention relates to a method for the preparation of the polypeptide of the invention, comprising
culturing a transformed cell of the present invention, which is capable of expressing the nucleic acid of the invention, under conditions that facilitate that the transformed cell expresses the nucleic acid fragment of the invention, which encodes a polypeptide of the invention, and subsequently recovering said polypeptide, or
preparing said polypeptide by means of solid or liquid phase peptide synthesis.

In a 15^{th} aspect, the invention relates to a method for determining whether a substance, such as an antibody, is potentially useful for treating infection with S. aureus, the method comprising contacting the polypeptide of the invention with the substance and subsequently establishing whether the substance has at least one of the following characteristics:
1) the ability to bind specifically to said polypeptide,
2) the ability to compeed with said polypeptide for specific binding to a ligand/receptor, and
3) the ability to specifically inactivate said polypeptide.

Finally, in a 16^{th} aspect, the invention relates to a method for determining whether a substance, such as a nucleic acid, is potentially useful for treating infection with S. aureus, the method comprising contacting the substance with the nucleic acid fragment of claim of the invention and subsequently establishing whether the substance has either the ability to
1) bind specifically to the nucleic acid fragment, or
2) bind specifically to a nucleic acid that hybridizes specifically with the nucleic acid fragment.

### LEGENDS TO THE FIGURES

FIG. 1: Survival plots for mice vaccinated with a polypeptide of the invention and a negative control, respectively. Solid line: negative control. Interrupted line: polypeptide of the invention.

FIG. 2: Survival plots for mice vaccinated with a polypeptide of the invention and a negative control, respectively. Solid line: negative control. Interrupted line: polypeptide of the invention.

FIG. 3: Survival plots for mice vaccinated with a polypeptide of the invention and a negative control, respectively. Solid line: negative control. Interrupted line: polypeptide of the invention.

FIG. 4: Survival plots for mice vaccinated with a polypeptide of the invention and a negative control, respectively. Solid line: negative control. Interrupted line: polypeptide of the invention.

FIG. 5: Survival plots for mice vaccinated with a polypeptide of the invention and a negative control, respectively. Interrupted line: negative control. Solid line: polypeptide of the invention.

FIG. 6: Survival plots for mice vaccinated with a polypeptide of the invention and a negative control, respectively. Interrupted line: negative control. Solid line: polypeptide of the invention.

FIG. 7: Survival plots for mice vaccinated with a polypeptide of the invention and a negative control, respectively. Interrupted line: negative control. Solid line: polypeptide of the invention.

### DETAILED DISCLOSURE OF THE INVENTION

### Definitions

The term “polypeptide” is in the present context intended to mean both short peptides of from 2 to 10 amino acid residues, oligopeptides of from 11 to 100 amino acid residues, and polypeptides of more than 100 amino acid residues. Further-more, the term is also intended to include proteins, i.e. functional biomolecules comprising at least one polypeptide; when comprising at least two polypeptides, these may form complexes, be covalently linked, or may be non-covalently linked. The polypeptide (s) in a protein can be glycosylated and/or lipidated and/or comprise prosthetic groups.

The term “subsequence” means any consecutive stretch of at least 3 amino acids or, when relevant, of at least 3 nucleotides, derived directly from a naturally occurring amino acid sequence or nucleic acid sequence, respectively

The term “amino acid sequence” s the order in which amino acid residues, connected by peptide bonds, lie in the chain in peptides and proteins.

The term “adjuvant” has its usual meaning in the art of vaccine technology, i.e. a substance or a composition of matter which is 1) not in itself capable of mounting a specific immune response against the immunogen of the vaccine, but which is 2) nevertheless capable of enhancing the immune response against the immunogen. Or, in other words, vaccination with the adjuvant alone does not provide an immune response against the immunogen, vaccination with the immunogen may or may not give rise to an immune response against the immunogen, but the combined vaccination with immunogen and adjuvant induces an immune response against the immunogen which is stronger than that induced by the immunogen alone.

“Sequence identity” is in the context of the present invention determined by comparing 2 optimally aligned sequences of equal length (e.g. DNA, RNA or amino acid)according to the following formula: (N_{ref}−N_{dif})·100/N_{ref}, wherein N_{ref} is the number of residues in one of the 2 sequences and N_{dif} is the number of residues which are non-identical in the two sequences when they are aligned over their entire lengths and in the same direction. So, two sequences 5′-ATTCGGAACC-3′ and 5′-ATACGGGACC-3′ will provide the sequence identity 80% (N_{ref}=10 and N_{dif}=2).

An “assembly of amino acids” means two or more amino acids bound together by physical or chemical means.

The “3D conformation” is the 3 dimensional structure of a biomolecule such as a protein. In monomeric polypeptides/proteins, the 3D conformation is also termed “the tertiary structure” and denotes the relative locations in 3 dimensional space of the amino acid residues forming the polypeptide.

“An immunogenic carrier” is a molecule or moiety to which an immunogen or a hapten can be coupled in order to enhance or enable the elicitation of an immune response against the immunogen/hapten. Immunogenic carriers are in classical cases relatively large molecules (such as tetanus toxoid, KLH, diphtheria toxoid etc.) which can be fused or conjugated to an immunogen/hapten, which is not sufficiently immunogenic in its own right—typically, the immunogenic carrier is capable of eliciting a strong T-helper lymphocyte response against the combined substance constituted by the immunogen and the immunogenic carrier, and this in turn provides for improved responses against the immungon by B-lymphocytes and cytotoxic lymphocytes. More recently, the large carrier molecules have to a certain extent been substituted by so-called promiscuous T-helper epitopes, i.e. shorter peptides that are recognized by a large fraction of HLA haplotypes in a population, and which elicit T-helper lymphocyte responses.

A “T-helper lymphocyte response” is an immune response elicited on the basis of a peptide, which is able to bind to an MHC class II molecule (e.g. an HLA class II molecule) in an antigen-presenting cell and which stimulates T-helper lymphocytes in an animal species as a consequence of T-cell receptor recognition of the complex between the peptide and the MHC Class II molecule prese

An “immunogen” is a substance of matter which is capable of inducing an adaptive immune response in a host, whose immune system is confronted with the immunogen. As such, immunogens are a subset of the larger genus “antigens”, which are substances that can be recognized specifically by the immune system (e.g. when bound by antibodies or, alternatively, when fragments of the are antigens bound to MHC molecules are being recognized by T-cell receptors) but which are not necessarily capable of inducing immunity -an antigen is, however, always capable of eliciting immunity, meaning that a host that has an established memory immunity against the antigen will mount a specific immune response against the antigen.

A “hapten” is a small molecule, which can neither induce or elicit an immune response, but if conjugated to an immunogenic carrier, antibodies or TCRs that recognize the hapten can be induced upon confrontation of the immune system with the hapten carrier conjugate.

An “adaptive immune response” is an immune response in response to confrontation with an antigen or immunogen, where the immune response is specific for antigenic determinants of the antigen/immunogen—examples of adaptive immune responses are induction of antigen specific antibody production or antigen specific induction/activation of T helper lymphocytes or cytotoxic lymphocytes.

A “protective, adaptive immune response” is an antigen-specific immune response induced in a subject as a reaction to immunization (artificial or natural) with an antigen, where the immune response is capable of protecting the subject against subsequent challenges with the antigen or a pathology-related agent that includes the antigen. Typically, prophylactic vaccination aims at establishing a protective adaptive immune response against one or several pathogens.

“Stimulation of the immune system” means that a substance or composition of matter exhibits a general, non-specific immunostimulatory effect. A number of adjuvants and putative adjuvants (such as certain cytokines) share the ability to stimulate the immune system. The result of using an immunostimulating agent is an increased “alertness” of the immune system meaning that simultaneous or subsequent immunization with an immunogen induces a significantly more effective immune response compared to isolated use of the immunogen.

Hybridization under “stringent conditions” is herein defined as hybridization performed under conditions by which a probe will hybridize to its target sequence, to a detectably greater degree than to other sequences. Stringent conditions are target-sequence-dependent and will differ depending on the structure of the polynucleotide. By controlling the stringency of the hybridization and/or washing conditions, target sequences can be identified which are 100% complementary to a probe (homologous probing). Alternatively, stringency conditions can be adjusted to allow some mismatching in sequences so that lower degrees of similarity are detected (heterologous probing). Specificity is typically the function of post-hybridization washes, the critical factors being the ionic strength and temperature of the final wash solution. Generally, stringent wash temperature conditions are selected to be about 5° C. to about 2° C. lower than the melting point (Tm) for the specific sequence at a defined ionic strength and pH. The melting point, or denaturation, of DNA occurs over a narrow temperature range and represents the disruption of the double helix into its complementary single strands. The process is described by the temperature of the midpoint of transition, Tm, which is also called the melting temperature. Formulas are available in the art for the determination of melting temperatures.

The term “animal” is in the present context in general intended to denote an animal species (preferably mammalian), such as Homo sapiens, Canis domesticus, etc. and not just one single animal. However, the term also denotes a population of such an animal species, since it is important that the individuals immunized according to the method of the invention substantially all will mount an immune response against the immunogen of the present invention.

As used herein, the term “antibody”refers to a polypeptide or group of polypeptides composed of at least one antibody combining site. An “antibody combining site” is the three-dimensional binding space with an internal surface shape and charge distribution complementary to the features of an epitope of an antigen, which allows a binding of the antibody with the antigen. “Antibody”includes, for example, vertebrate antibodies, hybrid antibodies, chimeric antibodies, humanised antibodies, altered antibodies, univalent antibodies, Fab proteins, and single domain antibodies.

“Specific binding” denotes binding between two substances which goes beyond binding of either substance to randomly chosen substances and also goes beyond simple association between substances that tend to aggregate because they share the same overall hydrophobicity or hydrophilicity. As such, specific binding usually involves a combination of electrostatic and other interactions between two conformationally complementary areas on the two substances, meaning that the substances can “recognize” each other in a complex mixture.

The term “vector” is used to refer to a carrier nucleic acid molecule into which a heterologous nucleic acid sequence can be inserted for introduction into a cell where it can be replicated and expressed. The term further denotes certain biological vehicles useful for the same purpose, e.g. viral vectors and phage—both these infectious agents are capable of introducing a heterelogous nucleic acid sequence

The term “expression vector” refers to a vector containing a nucleic acid sequence coding for at least part of a gene product capable of being transcribed. In some cases, when the transcription product is an mRNA molecule, this is in trun translated into a protein, polypeptide, or peptide.

### Specific Embodiments of the Invention

### The Polypeptides of the Invention

In some embodiments the at least 5 contiguous amino acids referred to in option b) in the definition of the first aspect of the invention constitute at least or exactly or at most 6 contiguous amino acid residues, such as at least or exactly or at most 7, at least or exactly or at most 8, at least or exactly or at most 9, at least or exactly or at most 10, at least or exactly or at most 11, at least or exactly or at most 12, at least or exactly or at most 13, at least or exactly or at most 14, at least or exactly or at most 15, at least or exactly or at most 16, at least or exactly or at most 17, at least or exactly or at most 18, at least or exactly or at most 19, at least or exactly or at most 20, at least or exactly or at most 21, at least or exactly or at most 22, at least or exactly or at most 23, at least or exactly or at most 24, at least or exactly or at most 25, at least or exactly or at most 26, at least or exactly or at most 27 at least or exactly or at most 28, at least or exactly or at most 29, at least or exactly or at most 30, at least or exactly or at most 31, at least or exactly or at most 32, at least or exactly or at most 33, at least or exactly or at most 34, at least or exactly or at most 35, at least or exactly or at most 36, at least or exactly or at most 37, at least or exactly or at most 38, at least or exactly or at most 39, at least or exactly or at most 40, at least or exactly or at most 41, at least or exactly or at most 42, at least or exactly or at most 43, at least or exactly or at most 44, at least or exactly or at most 45, at least or exactly or at most 46, at least or exactly or at most 47, at least or exactly or at most 48, at least or exactly or at most 49, at least or exactly or at most 50, at least or exactly or at most 51, at least or exactly or at most 52, at least or exactly or at most 53, at least or exactly or at most 54, at least or exactly or at most 55, at least or exactly or at most 56, at least or exactly or at most 57, at least or exactly or at most 58, at least or exactly or at most 59, at least or exactly or at most 60, at least or exactly or at most 61, at least or exactly or at most 62, at least or exactly or at most 63, at least or exactly or at most 64, at least or exactly or at most 65, at least or exactly or at most 66, at least or exactly or at most 67, at least or exactly or at most 68, at least or exactly or at most 69, at least or exactly or at most 70, at least or exactly or at most 71, at least or exactly or at most 72, at least or exactly or at most 73, at least or exactly or at most 74, at least or exactly or at most 75, at least or exactly or at most 76, at least or exactly or at most 77, at least or exactly or at most 78, at least or exactly or at most 79, at least or exactly or at most 80, at least or exactly or at most 81, at least or exactly or at most 82, at least or exactly or at most 83, at least or exactly or at most 84, at least or exactly or at most 85, at least or exactly or at most 86, at least or exactly or at most 87, at least or exactly or at most 88, at least or exactly or at most 89, at least or exactly or at most 90, at least or exactly or at most 91, at least or exactly or at most 92, at least or exactly or at most 93, at least or exactly or at most 94, at least or exactly or at most 95, at least or exactly or at most 96, at least or exactly or at most 97, at least or exactly or at most 98, at least or exactly or at most 99, at least or exactly or at most 100, at least or exactly or at most 101, at least or exactly or at most 102, at least or exactly or at most 103, at least or exactly or at most 104, at least or exactly or at most 105, at least or exactly or at most 106, at least or exactly or at most 107, at least or exactly or at most 108, at least or exactly or at most 109, at least or exactly or at most 110, at least or exactly or at most 111, at least or exactly or at most 112, at least or exactly or at most 113, at least or exactly or at most 114, at least or exactly or at most 115, at least or exactly or at most 116, at least or exactly or at most 117, at least or exactly or at most 118, at least or exactly or at most 119, at least or exactly or at most 120, at least or exactly or at most 121, at least or exactly or at most 122, at least or exactly or at most 123, at least or exactly or at most 124, at least or exactly or at most 125, at least or exactly or at most 126, at least or exactly or at most 127, at least or exactly or at most 128, at least or exactly or at most 129, at least or exactly or at most 130, at least or exactly or at most 131, at least or exactly or at most 132, at least or exactly or at most 133, at least or exactly or at most 134, at least or exactly or at most 135, at least or exactly or at most 136, at least or exactly or at most 137, at least or exactly or at most 138, and at least or exactly or at most 139 contiguous amino acid residues.

The number of contiguous amino acids can be higher, for all of SEQ ID NOs. 2-16 and 49. Another way to phrase this is that for each of SEQ ID NOs: 1-16 and 49, the number of the contiguous amino acid residues is at least or exactly or at most N−n, where N is the length of the sequence ID in question and n is any integer between N−5 and 0; that is, the at least 5 contiguous amino acids can be at least any number between 5 and the length of the reference sequence minus one, in increments of one. Consequently:

Insofar as embodiment b relates to SEQ ID NO: 2-16 and 49, the at least 5 contiguous amino acids referred to in option b) in the definition of the first aspect of the invention will preferably consititute at least or exactly or at most 140, at least or exactly or at most 141, at least or exactly or at most 142, at least or exactly or at most 143, at least or exactly or at most 144, at least or exactly or at most 145, at least or exactly or at most 146, at least or exactly or at most 147, at least or exactly or at most 148, at least or exactly or at most 149, at least or exactly or at most 150, at least or exactly or at most 151, at least or exactly or at most 152, at least or exactly or at most 153, at least or exactly or at most 154, at least or exactly or at most 155, at least or exactly or at most 156, at least or exactly or at most 157, at least or exactly or at most 158, at least or exactly or at most 159, at least or exactly or at most 160, at least or exactly or at most 161, at least or exactly or at most 162, at least or exactly or at most 163, at least or exactly or at most 164, at least or exactly or at most 165, at least or exactly or at most 166, at least or exactly or at most 167, at least or exactly or at most 168, at least or exactly or at most 169, at least or exactly or at most 170, at least or exactly or at most 171, at least or exactly or at most 172, at least or exactly or at most 173, at least or exactly or at most 174, at least or exactly or at most 175, at least or exactly or at most 176, at least or exactly or at most 177, at least or exactly or at most 178, at least or exactly or at most 179, at least or exactly or at most 180, at least or exactly or at most 181, at least or exactly or at most 182, at least or exactly or at most 183, at least or exactly or at most 184, at least or exactly or at most 185, at least or exactly or at most 186, at least or exactly or at most 187, at least or exactly or at most 188, at least or exactly or at most 189, at least or exactly or at most 190, at least or exactly or at most 191, or at least or exactly or at most 192 contiguous amino acid residues.

Insofar as embodiment b relates to SEQ ID NO: 3-16 and 49, the at least 5 contiguous amino acids referred to in option b) in the definition of the first aspect of the invention will preferably consititute at least or exactly or at most 193, at least or exactly or at most 194, at least or exactly or at most 195, at least or exactly or at most 196, at least or exactly or at most 197, at least or exactly or at most 198, at least or exactly or at most 199, at least or exactly or at most 200, at least or exactly or at most 201, at least or exactly or at most 202, at least or exactly or at most 203, at least or exactly or at most 204, at least or exactly or at most 205, at least or exactly or at most 206, at least or exactly or at most 207, or at least or exactly or at most 208 contiguous amino acid residues.

Insofar as embodiment b relates to SEQ ID NO: 4-16 and 49, the at least 5 contiguous amino acids referred to in option b) in the definition of the first aspect of the invention will preferably consititute at least or exactly or at most 209, at least or exactly or at most 210, at least or exactly or at most 211, at least or exactly or at most 212, at least or exactly or at most 213, at least or exactly or at most 214, at least or exactly or at most 215, at least or exactly or at most 216, at least or exactly or at most 217, at least or exactly or at most 218, at least or exactly or at most 219, at least or exactly or at most 220, at least or exactly or at most 221, at least or exactly or at most 222, at least or exactly or at most 223, at least or exactly or at most 224, at least or exactly or at most 225, at least or exactly or at most 226, at least or exactly or at most 227, at least or exactly or at most 228, at least or exactly or at most 229, at least or exactly or at most 230, at least or exactly or at most 231, at least or exactly or at most 232, at least or exactly or at most 233, at least or exactly or at most 234, at least or exactly or at most 235, at least or exactly or at most 236, at least or exactly or at most 237, at least or exactly or at most 238, at least or exactly or at most 239, at least or exactly or at most 240, or at least or exactly or at most 241 contiguous amino acid residues.

Insofar as embodiment b relates to SEQ ID NO: 5-16 and 49, the at least 5 contiguous amino acids referred to in option b) in the definition of the first aspect of the invention will preferably consititute at least or exactly or at most 242, at least or exactly or at most 243, at least or exactly or at most 244, at least or exactly or at most 245, at least or exactly or at most 246, at least or exactly or at most 247, at least or exactly or at most 248, at least or exactly or at most 249, at least or exactly or at most 250, at least or exactly or at most 251, at least or exactly or at most 252, at least or exactly or at most 253, at least or exactly or at most 254, at least or exactly or at most 255, at least or exactly or at most 256, at least or exactly or at most 257, at least or exactly or at most 258, at least or exactly or at most 259, at least or exactly or at most 260, at least or exactly or at most 261, at least or exactly or at most 262, at least or exactly or at most 263, at least or exactly or at most 264, at least or exactly or at most 265, at least or exactly or at most 266, at least or exactly or at most 267, at least or exactly or at most 268, at least or exactly or at most 269, at least or exactly or at most 270, at least or exactly or at most 271, at least or exactly or at most 272, at least or exactly or at most 273, at least or exactly or at most 274, at least or exactly or at most 275, at least or exactly or at most 276, at least or exactly or at most 277, at least or exactly or at most 278, at least or exactly or at most 279, at least or exactly or at most 280, at least or exactly or at most 281, at least or exactly or at most 282, at least or exactly or at most 283, at least or exactly or at most 284, at least or exactly or at most 285, at least or exactly or at most 286, at least or exactly or at most 287, at least or exactly or at most 288, at least or exactly or at most 289, at least or exactly or at most 290, at least or exactly or at most 291, at least or exactly or at most 292, at least or exactly or at most 293, at least or exactly or at most 294, at least or exactly or at most 295, at least or exactly or at most 296, at least or exactly or at most 297, at least or exactly or at most 298, at least or exactly or at most 299, at least or exactly or at most 300, at least or exactly or at most 301, at least or exactly or at most 302, at least or exactly or at most 303, at least or exactly or at most 304, at least or exactly or at most 305, at least or exactly or at most 306, at least or exactly or at most 307, at least or exactly or at most 308, at least or exactly or at most 309, at least or exactly or at most 310, at least or exactly or at most 311, at least or exactly or at most 312, at least or exactly or at most 313, at least or exactly or at most 314, at least or exactly or at most 315, at least or exactly or at most 316, at least or exactly or at most 317, at least or exactly or at most 318, at least or exactly or at most 319, at least or exactly or at most 320, at least or exactly or at most 321, at least or exactly or at most 322, at least or exactly or at most 323, at least or exactly or at most 324, at least or exactly or at most 325, or at least or exactly or at most 326 contiguous amino acid residues.

Insofar as embodiment b relates to SEQ ID NO: 6-16 and 49, the at least 5 contiguous amino acids referred to in option b) in the definition of the first aspect of the invention will preferably consititute at least or exactly or at most 327, at least or exactly or at most 328, at least or exactly or at most 329, at least or exactly or at most 330, at least or exactly or at most 331, at least or exactly or at most 332, at least or exactly or at most 333, at least or exactly or at most 334, at least or exactly or at most 335, at least or exactly or at most 336, at least or exactly or at most 337, at least or exactly or at most 338, at least or exactly or at most 339, at least or exactly or at most 340, at least or exactly or at most 341, at least or exactly or at most 342, at least or exactly or at most 343, at least or exactly or at most 344, at least or exactly or at most 345, at least or exactly or at most 346, at least or exactly or at most 347, at least or exactly or at most 348, at least or exactly or at most 349, at least or exactly or at most 350, at least or exactly or at most 351, at least or exactly or at most 352, at least or exactly or at most 353, at least or exactly or at most 354, at least or exactly or at most 355, or at least or exactly or at most 356 contiguous amino acid residues.

Insofar as embodiment b relates to SEQ ID NO: 7-16 and 49, the at least 5 contiguous amino acids referred to in option b) in the definition of the first aspect of the invention will preferably consititute at least or exactly or at most 357, at least or exactly or at most 358, at least or exactly or at most 359, at least or exactly or at most 360, at least or exactly or at most 361, at least or exactly or at most 362, at least or exactly or at most 363, at least or exactly or at most 364, at least or exactly or at most 365, at least or exactly or at most 366, at least or exactly or at most 367, at least or exactly or at most 368, at least or exactly or at most 369, at least or exactly or at most 370, at least or exactly or at most 371, at least or exactly or at most 372, at least or exactly or at most 373, at least or exactly or at most 374, at least or exactly or at most 375, at least or exactly or at most 376, at least or exactly or at most 377, at least or exactly or at most 378, at least or exactly or at most 379, at least or exactly or at most 380, at least or exactly or at most 381, at least or exactly or at most 382, at least or exactly or at most 383, at least or exactly or at most 384, at least or exactly or at most 385, at least or exactly or at most 386, at least or exactly or at most 387, at least or exactly or at most 388, at least or exactly or at most 389, at least or exactly or at most 390, at least or exactly or at most 391, at least or exactly or at most 392, at least or exactly or at most 393, at least or exactly or at most 394, at least or exactly or at most 395, at least or exactly or at most 396, at least or exactly or at most 397, or at least or exactly or at most 398 contiguous amino acid residues.

Insofar as embodiment b relates to SEQ ID NO: 8-18 and 49, the at least 5 contiguous amino acids referred to in option b) in the definition of the first aspect of the invention will preferably consititute at least or exactly or at most 399, at least or exactly or at most 400, at least or exactly or at most 401, at least or exactly or at most 402, at least or exactly or at most 403, at least or exactly or at most 404, at least or exactly or at most 405, at least or exactly or at most 406, or at least or exactly or at most 407 contiguous amino acid residues.

Insofar as embodiment b relates to SEQ ID NO: 8-18, the at least 5 contiguous amino acids referred to in option b) in the definition of the first aspect of the invention will preferably consititute at least or exactly or at most 408, at least or exactly or at most 409, at least or exactly or at most 410, at least or exactly or at most 411, at least or exactly or at most 412, at least or exactly or at most 413, at least or exactly or at most 414, at least or exactly or at most 415, at least or exactly or at most 416, at least or exactly or at most 417, at least or exactly or at most 418, at least or exactly or at most 419, at least or exactly or at most 420, at least or exactly or at most 421, at least or exactly or at most 422, at least or exactly or at most 423, at least or exactly or at most 424, at least or exactly or at most 425, at least or exactly or at most 426, at least or exactly or at most 427, at least or exactly or at most 428, at least or exactly or at most 429, at least or exactly or at most 430, at least or exactly or at most 431, at least or exactly or at most 432, at least or exactly or at most 433, at least or exactly or at most 434, at least or exactly or at most 435, at least or exactly or at most 436, at least or exactly or at most 437, at least or exactly or at most 438, at least or exactly or at most 439, at least or exactly or at most 440, at least or exactly or at most 441, at least or exactly or at most 442, at least or exactly or at most 443, at least or exactly or at most 444, at least or exactly or at most 445, at least or exactly or at most 446, at least or exactly or at most 447, at least or exactly or at most 448, at least or exactly or at most 449, at least or exactly or at most 450, at least or exactly or at most 451, at least or exactly or at most 452, at least or exactly or at most 453, at least or exactly or at most 454, at least or exactly or at most 455, at least or exactly or at most 456, at least or exactly or at most 457, at least or exactly or at most 458, at least or exactly or at most 459, at least or exactly or at most 460, at least or exactly or at most 461, at least or exactly or at most 462, at least or exactly or at most 463, at least or exactly or at most 464, at least or exactly or at most 465, at least or exactly or at most 466, at least or exactly or at most 467, at least or exactly or at most 468, at least or exactly or at most 469, at least or exactly or at most 470, at least or exactly or at most 471, at least or exactly or at most 472, at least or exactly or at most 473, at least or exactly or at most 474, at least or exactly or at most 475, at least or exactly or at most 476, at least or exactly or at most 477, at least or exactly or at most 478, at least or exactly or at most 479, at least or exactly or at most 480, at least or exactly or at most 481, at least or exactly or at most 482, at least or exactly or at most 483, at least or exactly or at most 484, at least or exactly or at most 485, at least or exactly or at most 486, at least or exactly or at most 487, at least or exactly or at most 488, at least or exactly or at most 489, at least or exactly or at most 490, at least or exactly or at most 491, at least or exactly or at most 492, at least or exactly or at most 493, or at least or exactly or at most 494 contiguous amino acid residues.

Insofar as embodiment b relates to SEQ ID NO: 9-18, the at least 5 contiguous amino acids referred to in option b) in the definition of the first aspect of the invention will preferably consititute at least or exactly or at most 495, at least or exactly or at most 496, at least or exactly or at most 497, at least or exactly or at most 498, at least or exactly or at most 499, at least or exactly or at most 500, at least or exactly or at most 501, at least or exactly or at most 502, at least or exactly or at most 503, at least or exactly or at most 504, at least or exactly or at most 505, at least or exactly or at most 506 contiguous amino acid residues.

Insofar as embodiment b relates to SEQ ID NO: 10-18, the at least 5 contiguous amino acids referred to in option b) in the definition of the first aspect of the invention will preferably consititute at least or exactly or at most 507 contiguous amino acid residues or at least or exactly or at most 508 contiguous amino acid residues.

Insofar as embodiment b relates to SEQ ID NO: 11-18, the at least 5 contiguous amino acids referred to in option b) in the definition of the first aspect of the invention will preferably consititute at least or exactly or at most 509, at least or exactly or at most 510, at least or exactly or at most 511, at least or exactly or at most 512, at least or exactly or at most 513, or at least or exactly or at most 514 contiguous amino acid residues.

Insofar as embodiment b relates to SEQ ID NO: 12-18, the at least 5 contiguous amino acids referred to in option b) in the definition of the first aspect of the invention will preferably consititute at least or exactly or at most 515, at least or exactly or at most 516, at least or exactly or at most 517, at least or exactly or at most 518, at least or exactly or at most 519, at least or exactly or at most 520, at least or exactly or at most 521, at least or exactly or at most 522, at least or exactly or at most 523, at least or exactly or at most 524, at least or exactly or at most 525, at least or exactly or at most 526, at least or exactly or at most 527, at least or exactly or at most 528, at least or exactly or at most 529, at least or exactly or at most 530, at least or exactly or at most 531, at least or exactly or at most 532, at least or exactly or at most 533, at least or exactly or at most 534, at least or exactly or at most 535, at least or exactly or at most 536, at least or exactly or at most 537, at least or exactly or at most 538, at least or exactly or at most 539, at least or exactly or at most 540, at least or exactly or at most 541, at least or exactly or at most 542, at least or exactly or at most 543, at least or exactly or at most 544, at least or exactly or at most 545, at least or exactly or at most 546, at least or exactly or at most 547, at least or exactly or at most 548, at least or exactly or at most 549, at least or exactly or at most 550, at least or exactly or at most 551, at least or exactly or at most 552, at least or exactly or at most 553, at least or exactly or at most 554, at least or exactly or at most 555, at least or exactly or at most 556, at least or exactly or at most 557, at least or exactly or at most 558, at least or exactly or at most 559, at least or exactly or at most 560, at least or exactly or at most 561, at least or exactly or at most 562, or at least or exactly or at most 563 contiguous amino acid residues.

Insofar as embodiment b relates to SEQ ID NO: 13-18, the at least 5 contiguous amino acids referred to in option b) in the definition of the first aspect of the invention will preferably consititute at least or exactly or at most 564, at least or exactly or at most 565, at least or exactly or at most 566, at least or exactly or at most 567, at least or exactly or at most 568, at least or exactly or at most 569, at least or exactly or at most 570, at least or exactly or at most 571, at least or exactly or at most 572, at least or exactly or at most 573, at least or exactly or at most 574, at least or exactly or at most 575, at least or exactly or at most 576, at least or exactly or at most 577, at least or exactly or at most 578, at least or exactly or at most 579, at least or exactly or at most 580, at least or exactly or at most 581, at least or exactly or at most 582, at least or exactly or at most 583, at least or exactly or at most 584, at least or exactly or at most 585, at least or exactly or at most 586, at least or exactly or at most 587, at least or exactly or at most 588, at least or exactly or at most 589, at least or exactly or at most 590, at least or exactly or at most 591, at least or exactly or at most 592,at least or exactly or at most 593, at least or exactly or at most 594, at least or exactly or at most 595, at least or exactly or at most 596, at least or exactly or at most 597, at least or exactly or at most 598, at least or exactly or at most 599, at least or exactly or at most 600, at least or exactly or at most 601, at least or exactly or at most 602, at least or exactly or at most 603, at least or exactly or at most 604, at least or exactly or at most 605, at least or exactly or at most 606, at least or exactly or at most 607, at least or exactly or at most 608, at least or exactly or at most 609, at least or exactly or at most 610, at least or exactly or at most 611, at least or exactly or at most 612, at least or exactly or at most 613, at least or exactly or at most 614, at least or exactly or at most 615, at least or exactly or at most 616, at least or exactly or at most 617, or at least or exactly or at most 618 contiguous amino acid residues.

Insofar as embodiment b relates to SEQ ID NO: 14-18, the at least 5 contiguous amino acids referred to in option b) in the definition of the first aspect of the invention will preferably consititute at least or exactly or at most 619, at least or exactly or at most 620, at least or exactly or at most 621, at least or exactly or at most 622, at least or exactly or at most 623, at least or exactly or at most 624, at least or exactly or at most 625, at least or exactly or at most 626, at least or exactly or at most 627, at least or exactly or at most 628, at least or exactly or at most 629, at least or exactly or at most 630, at least or exactly or at most 631, at least or exactly or at most 632, at least or exactly or at most 633, at least or exactly or at most 634, at least or exactly or at most 635, at least or exactly or at most 636, at least or exactly or at most 637, at least or exactly or at most 638, at least or exactly or at most 639, at least or exactly or at most 640, at least or exactly or at most 641, at least or exactly or at most 642, at least or exactly or at most 643, at least or exactly or at most 644, at least or exactly or at most 645, at least or exactly or at most 646, at least or exactly or at most 647, at least or exactly or at most 648, at least or exactly or at most 649, at least or exactly or at most 650, at least or exactly or at most 651, at least or exactly or at most 652, at least or exactly or at most 653, at least or exactly or at most 654, at least or exactly or at most 655, at least or exactly or at most 656, at least or exactly or at most 657, at least or exactly or at most 658, at least or exactly or at most 659, at least or exactly or at most 660, at least or exactly or at most 661, at least or exactly or at most 662, at least or exactly or at most 663, at least or exactly or at most 664, at least or exactly or at most 665, at least or exactly or at most 666, at least or exactly or at most 667, at least or exactly or at most 668, at least or exactly or at most 669, at least or exactly or at most 670, at least or exactly or at most 671, at least or exactly or at most 672, at least or exactly or at most 673, at least or exactly or at most 674, at least or exactly or at most 675, at least or exactly or at most 676, at least or exactly or at most 677, at least or exactly or at most 678, at least or exactly or at most 679, or at least or exactly or at most 680 contiguous amino acid residues.

Insofar as embodiment b relates to SEQ ID NO: 15-18, the at least 5 contiguous amino acids referred to in option b) in the definition of the first aspect of the invention will preferably consititute at least or exactly or at most 681, at least or exactly or at most 682, at least or exactly or at most 683, at least or exactly or at most 684, at least or exactly or at most 685, at least or exactly or at most 686, at least or exactly or at most 687, at least or exactly or at most 688, at least or exactly or at most 689, at least or exactly or at most 690, at least or exactly or at most 691, at least or exactly or at most 692, at least or exactly or at most 693, at least or exactly or at most 694, at least or exactly or at most 695, at least or exactly or at most 696, at least or exactly or at most 697, at least or exactly or at most 698, at least or exactly or at most 699, at least or exactly or at most 700, at least or exactly or at most 701, at least or exactly or at most 702, at least or exactly or at most 703, at least or exactly or at most 704, at least or exactly or at most 705, at least or exactly or at most 706, at least or exactly or at most 707, at least or exactly or at most 708, at least or exactly or at most 709, at least or exactly or at most 710, at least or exactly or at most 711, at least or exactly or at most 712, at least or exactly or at most 713, at least or exactly or at most 714, at least or exactly or at most 715, at least or exactly or at most 716, at least or exactly or at most 717, at least or exactly or at most 718, at least or exactly or at most 719, at least or exactly or at most 720, at least or exactly or at most 721, at least or exactly or at most 722, at least or exactly or at most 723, at least or exactly or at most 724, at least or exactly or at most 725, at least or exactly or at most 726, at least or exactly or at most 727, at least or exactly or at most 728, at least or exactly or at most 729, at least or exactly or at most 730, at least or exactly or at most 731, at least or exactly or at most 732, at least or exactly or at most 733, at least or exactly or at most 734, at least or exactly or at most 735, at least or exactly or at most 736, at least or exactly or at most 737, at least or exactly or at most 738, at least or exactly or at most 739, at least or exactly or at most 740, at least or exactly or at most 741, at least or exactly or at most 742, at least or exactly or at most 743, at least or exactly or at most 744, at least or exactly or at most 745, at least or exactly or at most 746, at least or exactly or at most 747, at least or exactly or at most 748, at least or exactly or at most 749, at least or exactly or at most 750, at least or exactly or at most 751, at least or exactly or at most 752, at least or exactly or at most 753, at least or exactly or at most 754, at least or exactly or at most 755, at least or exactly or at most 756, at least or exactly or at most 757, at least or exactly or at most 758, at least or exactly or at most 759, at least or exactly or at most 760, at least or exactly or at most 761, at least or exactly or at most 762, at least or exactly or at most 763, at least or exactly or at most 764, at least or exactly or at most 765, at least or exactly or at most 766, at least or exactly or at most 767, at least or exactly or at most 768, at least or exactly or at most 769, at least or exactly or at most 770, at least or exactly or at most 771, at least or exactly or at most 772, at least or exactly or at most 773, at least or exactly or at most 774, at least or exactly or at most 775, at least or exactly or at most 776, at least or exactly or at most 777, at least or exactly or at most 778, at least or exactly or at most 779, at least or exactly or at most 780, at least or exactly or at most 781, at least or exactly or at most 782, at least or exactly or at most 783, at least or exactly or at most 784, at least or exactly or at most 785, at least or exactly or at most 786, at least or exactly or at most 787, at least or exactly or at most 788, at least or exactly or at most 789, at least or exactly or at most 790, at least or exactly or at most 791, at least or exactly or at most 792, at least or exactly or at most 793, at least or exactly or at most 794, at least or exactly or at most 795, at least or exactly or at most 796, at least or exactly or at most 797, at least or exactly or at most 798, at least or exactly or at most 799, at least or exactly or at most 800, at least or exactly or at most 801, at least or exactly or at most 802, at least or exactly or at most 803, at least or exactly or at most 804, or at least or exactly or at most 805 contiguous amino acid residues.

Insofar as embodiment b relates to SEQ ID NO: 16, the at least 5 contiguous amino acids referred to in option b) in the definition of the first aspect of the invention will preferably consititute at least or exactly or at most 806 contiguous amino acid residues or at least or exactly or at most 807 contiguous amino acid residues.

In some embodiments, the polypeptide of the invention also has a sequence identity with the amino acid sequence of a) defined above of at least 65%, such as at least 70%, at least 75%, at least 80%, at least 85%, at least 90%, at least 91%, at least 92%, at least 93%, at least 94%, at least 95%, at least 96%, at least 97%, at least 98%, and at least 99%. Similarly, the polypeptide of the invention in some embodiments also has a sequence identity with the amino acid sequence of b) defined above of at least 60%, such as at least 65%, at least 70%, at least 75%, at least 80%, at least 85%, at least 90%, at least 91%, at least 92%, at least 93%, at least 94%, at least 95%, at least 96%, at least 97%, at least 98%, and at least 99%.

In the embodiments defined by option b) above, the polypeptide of the invention is also one that has at least or exactly or at most 5 contiguous amino acid residues defined for option b) above and also has its N-terminal amino acid residue corresponding to any one of amino acid residues 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40,42, 43, 44, 45, 46, 47, 48, 49, 15 50, 51, 52, 53, 54, 55, 56, 57, 58, 59, 60, 61, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 110, 111, 112, 113, 114, 115, 116, 117, 118, 119, 120, 121, 122, 123, 124, 125, 126, 127, 128, 129, 130, 131, 132, 133, 134, 135, 136 in any one of SEQ ID NOs: 1-16, if the length of the at least or exactly or at most 5 amino acid residues so permit—if the length of the at least or exactly or at most 5 amino acids is higher than 5, the N-terminal first residue will not be higher numbered than N−L+1, where N is the number of amino acid residues of the reference sequence and L is the number of amino acids defined for option b.

In the embodiments defined by option b) above, the polypeptide of the invention is also one that has at least or exactly or at most 5 contiguous amino acid residues defined for option b) above and also has its N-terminal amino acid residue corresponding to any one of amino acid residues 137, 138, 139, 140, 141, 142, 143, 144, 145, 146, 147, 148, 149, 150, 151, 152, 153, 154, 155, 156, 157, 158, 159, 160, 171, 172, 173, 174, 175, 176, 177, 178, 179, 180, 181, 182, 183, 184, 185, 186, 187, 188 and 189 in any on of SEQ ID NOs: 2-16, if the length of the at least or exactly or at most 5 amino acid residues so permit—if the length of the at least or exactly or at most 5 amino acids is higher than 5, the N-terminal first residue will not be higher numbered than N−L+1, where N is the number of amino acid residues of the reference sequence and L is the number of amino acids defined for option b.

In the embodiments defined by option b) above, the polypeptide of the invention is also one that has at least or exactly or at most 5 contiguous amino acid residues defined for option b) above and also has its N-terminal amino acid residue corresponding to any one of amino acid residues 190, 191, 192, 193, 194, 195, 196, 197, 198, 199, 200, 201, 202, 203, 204 and 205 in any one of SEQ ID NOs: 3-16, if the length of the at least or exactly or at most 5 amino acid residues so permit—if the length of the at least or exactly or at most 5 amino acids is higher than 5, the N-terminal first residue will not be higher numbered than N−L+1, where N is the number of amino acid residues of the reference sequence and L is the number of amino acids defined for option b.

In the embodiments defined by option b) above, the polypeptide of the invention is also one that has at least or exactly or at most 5 contiguous amino acid residues defined for option b) above and also has its N-terminal amino acid residue corresponding to any one of amino acid residues 206, 207, 208, 209, 210, 211, 212, 213, 214, 215, 216, 217, 218, 219, 220, 221, 222, 223, 224, 225, 226, 227, 228, 229, 230, 231, 232, 233, 234, 235, 236, 237 and 238 in any one of SEQ ID NOs: 4-16, if the length of the at least or exactly or at most 5 amino acid residues so permit—if the length of the at least or exactly or at most 5 amino acids is higher than 5, the N-terminal first residue will not be higher numbered than N−L+1, where N is the number of amino acid residues of the reference sequence and L is the number of amino acids defined for option b.

In the embodiments defined by option b) above, the polypeptide of the invention is also one that has at least or exactly or at most 5 contiguous amino acid residues defined for option b) above and also has its N-terminal amino acid residue corresponding to any one of amino acid residues 239, 240, 241, 242, 243, 244, 245, 246, 247, 248, 249, 250, 251, 252, 253, 254, 255, 256, 257, 258, 259, 260, 271, 272, 273, 274, 275, 276, 277, 278, 279, 280, 281, 282, 283, 284, 285, 286, 287, 288, 289, 290, 291, 292, 293, 294, 295, 296, 297, 298, 299, 300, 301, 302, 303, 304, 305, 306, 307, 308, 309, 310, 310, 311, 312, 313, 314, 315, 316, 317, 318, 319, 320, 321, 322 and 323 in any one of SEQ ID NOs: 5-16, if the length of the at least or exactly or at most 5 amino acid residues so permit—if the length of the at least or exactly or at most 5 amino acids is higher than 5, the N-terminal first residue will not be higher numbered than N−L+1, where N is the number of amino acid residues of the reference sequence and L is the number of amino acids defined for option b.

In the embodiments defined by option b) above, the polypeptide of the invention is also one that has at least or exactly or at most 5 contiguous amino acid residues defined for option b) above and also has its N-terminal amino acid residue corresponding to any one of amino acid residues 324, 325, 326, 327, 328, 329, 330, 331, 332, 333, 334, 335, 336, 337, 338, 339, 340, 341, 342, 343, 344, 345, 346, 347, 348, 349, 350, 351, 352 and 353 in any one of SEQ ID NOs: 6-16, if the length of the at least or exactly or at most 5 amino acid residues so permit—if the length of the at least or exactly or at most 5 amino acids is higher than 5, the N-terminal first residue will not be higher numbered than N−L+1, where N is the number of amino acid residues of the reference sequence and L is the number of amino acids defined for option b.

In the embodiments defined by option b) above, the polypeptide of the invention is also one that has at least or exactly or at most 5 contiguous amino acid residues defined for option b) above and also has its N-terminal amino acid residue corresponding to any one of amino acid residues 354, 355, 356, 357, 358, 359, 360, 361, 362, 363, 364, 365, 366, 367, 368, 369, 370, 371, 372, 373, 374, 375, 376, 377, 378, 379, 380, 381, 382, 383, 384, 385, 386, 387, 388, 389, 390, 391, 392, 393, 394 and 395 in any one of SEQ ID NOs: 7-16, if the length of the at least or exactly or at most 5 amino acid residues so permit—if the length of the at least or exactly or at most 5 amino acids is higher than 5, the N-terminal first residue will not be higher numbered than N−L+1, where N is the number of amino acid residues of the reference sequence and L is the number of amino acids defined for option b.

In the embodiments defined by option b) above, the polypeptide of the invention is also one that has at least or exactly or at most 5 contiguous amino acid residues defined for option b) above and also has its N-terminal amino acid residue corresponding to any one of amino acid residues 396, 397, 398, 399, 400, 401, 402, 403, 404, 405, 406, 407, 408, 409, 410, 411, 412, 413, 414, 415, 416, 417, 418, 419, 420, 421, 422, 423, 424, 425, 426, 427, 428, 429, 430, 431, 432, 433, 434, 435, 436, 437, 438, 439, 440, 441, 442, 443, 444, 445, 446, 447, 448, 449, 450, 451, 452, 453, 454, 455, 456, 457, 458, 459, 460, 461, 462, 463, 464, 465, 466, 467, 468, 469, 470, 471, 472, 473, 474, 475, 476, 477, 478, 479, 480, 481, 482, 483, 484, 485, 486, 487, 488, 489, 490 and 491 in any one of SEQ ID NOs: 8-16, if the length of the at least or exactly or at most 5 amino acid residues so permit—if the length of the at least or exactly or at most 5 amino acids is higher than 5, the N-terminal first residue will not be higher numbered than N−L+1, where N is the number of amino acid residues of the reference sequence and L is the number of amino acids defined for option b.

In the embodiments defined by option b) above, the polypeptide of the invention is also one that has at least or exactly or at most 5 contiguous amino acid residues defined for option b) above and also has its N-terminal amino acid residue corresponding to any one of amino acid residues 492, 493, 494, 495, 496, 497, 498, 499, 500, 501, 502 and 503 in any one of SEQ ID NOs: 9-16, if the length of the at least or exactly or at most 5 amino acid residues so permit—if the length of the at least or exactly or at most 5 amino acids is higher than 5, the N-terminal first residue will not be higher numbered than N−L+1, where N is the number of amino acid residues of the reference sequence and L is the number of amino acids defined for option b.

In the embodiments defined by option b) above, the polypeptide of the invention is also one that has at least or exactly or at most 5 contiguous amino acid residues defined for option b) above and also has its N-terminal amino acid residue corresponding to any one of amino acid residues 504 and 505 in any one of SEQ ID NOs: 10-16, if the length of the at least or exactly or at most 5 amino acid residues so permit—if the length of the at least or exactly or at most 5 amino acids is higher than 5, the N-terminal first residue will not be higher numbered than N−L+1, where N is the number of amino acid residues of the reference sequence and L is the number of amino acids defined for option b.

In the embodiments defined by option b) above, the polypeptide of the invention is also one that has at least or exactly or at most 5 contiguous amino acid residues defined for option b) above and also has its N-terminal amino acid residue corresponding to any one of amino acid residues 506, 507, 508, 509, 510 and 511 in any one of SEQ ID NOs: 11-16, if the length of the at least or exactly or at most 5 amino acid residues so permit—if the length of the at least or exactly or at most 5 amino acids is higher than 5, the N-terminal first residue will not be higher numbered than N−L+1, where N is the number of amino acid residues of the reference sequence and L is the number of amino acids defined for option b.

In the embodiments defined by option b) above, the polypeptide of the invention is also one that has at least or exactly or at most 5 contiguous amino acid residues defined for option b) above and also has its N-terminal amino acid residue corresponding to any one of amino acid residues 512, 513, 514, 515, 516, 517, 518, 519, 520, 521, 522, 523, 524, 525, 526, 527, 528, 529, 530, 531, 532, 533, 534, 535, 536, 537, 538, 539, 540, 541, 542, 543, 544, 545, 546, 547, 548, 549, 550, 551, 552, 553, 554, 555, 556, 557, 558, 559, 560 and 561 in any one of SEQ ID NOs: 12-16, if the length of the at least or exactly or at most 5 amino acid residues so permit—if the length of the at least or exactly or at most 5 amino acids is higher than 5, the N-terminal first residue will not be higher numbered than N−L+1, where N is the number of amino acid residues of the reference sequence and L is the number of amino acids defined for option b.

In the embodiments defined by option b) above, the polypeptide of the invention is also one that has at least or exactly or at most 5 contiguous amino acid residues defined for option b) above and also has its N-terminal amino acid residue corresponding to any one of amino acid residues 562, 563, 564, 565, 566, 567, 568, 569, 570, 571, 572, 573, 574, 575, 576, 577, 578, 579, 580, 581, 582, 583, 584, 585, 586, 587, 588, 589, 590, 591, 592, 593, 594, 595, 596, 597, 598, 599, 600, 601, 602, 603, 604, 605, 606, 607, 608, 609, 610, 611, 612, 613, 614 and 615 in any one of SEQ ID NOs: 13-16, if the length of the at least or exactly or at most 5 amino acid residues so permit—if the length of the at least or exactly or at most 5 amino acids is higher than 5, the N-terminal first residue will not be higher numbered than N−L+1, where N is the number of amino acid residues of the reference sequence and L is the number of amino acids defined for option b.

In the embodiments defined by option b) above, the polypeptide of the invention is also one that has at least or exactly or at most 5 contiguous amino acid residues defined for option b) above and also has its N-terminal amino acid residue corresponding to any one of amino acid residues 616, 617, 618, 619, 620, 621, 622, 623, 624, 625, 626, 627, 628, 629, 630, 631, 632, 633, 634, 635, 636, 637, 638, 639, 640, 641, 642, 643, 644, 645, 646, 647, 648, 649, 650, 651, 652, 653, 654, 655, 656, 657, 658, 659, 660, 661, 662, 663, 664, 665, 666, 667, 668, 669, 670, 671, 672, 673, 674, 675, 676 and 677 in SEQ ID NOs: 14-16, if the length of the at least or exactly or at most 5 amino acid residues so permit—if the length of the at least or exactly or at most 5 amino acids is higher than 5, the N-terminal first residue will not be higher numbered than N−L+1, where N is the number of amino acid residues of the reference sequence and L is the number of amino acids defined for option b.sss

In the embodiments defined by option b) above, the polypeptide of the invention is also one that has at least or exactly or at most 5 contiguous amino acid residues defined for option b) above and also has its N-terminal amino acid residue corresponding to any one of amino acid residues 678, 679, 680, 681, 682, 683, 684, 685, 686, 687, 688, 689, 690, 691, 692, 693, 694, 695, 696, 697, 698, 699, 700, 701, 702, 703, 704, 705, 706, 707, 708, 709, 710, 711, 712, 713, 714, 715, 716, 717, 718, 719, 720, 721, 722, 723, 724, 725, 726, 727, 728, 729, 15 730, 731, 732, 733, 734, 735, 736, 737, 738, 739, 740, 741, 742, 743, 744, 745, 746, 747, 748, 749, 750, 751, 752, 753, 754, 755, 756, 757, 758, 759, 760, 761, 762, 763, 764, 765, 766, 767, 768, 769, 770, 771, 772, 773, 774, 775, 776, 777, 778, 779, 780, 781, 782, 783, 784, 785, 786, 787, 788, 789, 790, 791, 792, 793, 794, 795, 796, 797, 798, 799, 800, 801 and 802 in SEQ ID NOs: 15-16, if the length of the at least or exactly or at most 5 amino acid residues so permit—if the length of the at least or exactly or at most 5 amino acids is higher than 5, the N-terminal first residue will not be higher numbered than N−L+1, where N is the number of amino acid residues of the reference sequence and L is the number of amino acids defined for option b.

In the embodiments defined by option b) above, the polypeptide of the invention is also one that has at least or exactly or at most 5 contiguous amino acid residues defined for option b) above and also has its N-terminal amino acid residue corresponding to any one of amino acid residues 803 and 804 in SEQ ID NO: 16, if the length of the at least or exactly or at most 5 amino acid residues so permit—if the length of the at least or exactly or at most 5 amino acids is higher than 5, the N-terminal first residue will not be higher numbered than N−L+1, where N is the number of amino acid residues of the reference sequence and L is the number of amino acids defined for option b.

The polypeptide of the invention is in certain embodiments also fused or conjugated to an immunogenic carrier molecule; or, phrased otherwise, the polypeptide of the invention also includes such an immunogenic carrier molecule in addition to the material derived from SEQ ID NOs. 1-16. The immunogenic carrier molecule is a typically polypeptide that induces T-helper lymphocyte responses in a majority of humans, such as immunogenic carrier proteins selected from the group consisting of keyhole limpet hemocyanino or a fragment thereof, tetanus toxoid or a fragment thereof, dipththeria toxoid or a fragment thereof. Other suitable carrier molecules are discussed infra. One further fusion partner, which is preferably incorporated is a “His tag”, i.e. a stretch of amino acids, which is rich or only consists of histidinyl residues so as to facilitate protein purification.

In preferred embodiments, the polypeptide of the invention detailed above is capable of inducing an adaptive immune response against the polypeptide in a mammal, in particular in a human being. Preferably, the adaptive immune response is a protective adaptive immune response against infection with S. aureus, in particular multi-resistant S. aureus. The polypeptide may in these cases induce a humeral and/or a cellular immune response.

A particularly preferred polypeptide of the invention is derived from SEQ ID NO: 13 and is otherwise as defined above.

### Epitopes

SEQ ID NOs: 1-16 include antigenic determinants (epitopes) that are as such recognized by antibodies and/or when bound to MHC molecules by T-cell receptors. For the purposes of the present invention, B-cell epitopes (i.e. antibody binding epitopes) are of particular relevance.

It is relatively uncomplicated to identify linear B-cell epitopes—one very simple approach entails that antibodies raised agains S. aureus or S. aureus derived proteins disclosed herein are tested for binding to overlapping oligomeric peptides derived from any one of SEQ ID NO: 1-16. Thereby, the regions of the S. aureus polypeptide which are responsible for or contribute to binding to the antibodies can be identified.

Alternatively, or additionally, one can produce mutated versions of the polypeptides of the invention, e.g. version where each single non-alanine residue in SEQ ID NOs.: 1-16 are point mutated to alanine—this method also assists in identifying complex assembled B-cell epitopes; this is the case when binding of the same antibody is modified by exchanging amino acids in different areas of the full-length polypeptide.

Also, in silico methods for B-cell epitope prediction can be employed: useful state-of-the-art systems for β-turn prediction is provided in Petersen B et al. (November 2010), Plos One 5(11): e15079; prediction of linear B-cell epitopes, cf: Larsen J E P et al. (April 2006), Immunome Research, 2:2; prediction of solvent exposed amino acids: Petersen B et al (July 2009), BMC Structural Biology, 9:51.

### The Nucleic Acid Fragments of the Invention

The nucleic acid fragment of the invention referred to above is preferably is a DNA fragment (such as SEQ ID NOs: 17-32 and 50) or an RNA fragment (such as SEQ ID NOs 33-48 and 51).

The nucleic acid fragment of the invention typically consists of at least or exactly or at most 11, such as at least or exactly or at most 12, at least or exactly or at most 13, at least or exactly or at most 14, at least or exactly or at most 15, at least or exactly or at most 16, at least or exactly or at most 17 at least or exactly or at most 18, at least or exactly or at most 19, at least or exactly or at most 20, at least or exactly or at most 21, at least or exactly or at most 22, at least or exactly or at most 23, at least or exactly or at most 24, at least or exactly or at most 25, at least or exactly or at most 26, at least or exactly or at most 27, at least or exactly or at most 28, at least or exactly or at most 29, at least or exactly or at most 30, at least or exactly or at most 31, at least or exactly or at most 32, at least or exactly or at most 33, at least or exactly or at most 34, at least or exactly or at most 35, at least or exactly or at most 36, at least or exactly or at most 37, at least or exactly or at most 38, at least or exactly or at most 39, at least or exactly or at most 40, at least or exactly or at most 41, at least or exactly or at most 42, at least or exactly or at most 43, at least or exactly or at most 44, at least or exactly or at most 45, at least or exactly or at most 46, at least or exactly or at most 47, at least or exactly or at most 48, at least or exactly or at most 49, at least or exactly or at most 50, at least or exactly or at most 51, at least or exactly or at most 52, at least or exactly or at most 53, at least or exactly or at most 54, at least or exactly or at most 55, at least or exactly or at most 56, at least or exactly or at most 57, at least or exactly or at most 58, at least or exactly or at most 59, at least or exactly or at most 60, at least or exactly or at most 61, at least or exactly or at most 62, at least or exactly or at most 63, at least or exactly or at most 64, at least or exactly or at most 65, at least or exactly or at most 66, at least or exactly or at most 67, at least or exactly or at most 68, at least or exactly or at most 69, at least or exactly or at most 70, at least or exactly or at most 71, at least or exactly or at most 72, at least or exactly or at most 73, at least or exactly or at most 74, at least or exactly or at most 75, at least or exactly or at most 76, at least or exactly or at most 77, at least or exactly or at most 78, at least or exactly or at most 79, at least or exactly or at most 80, at least or exactly or at most 81, at least or exactly or at most 82, at least or exactly or at most 83, at least or exactly or at most 84, at least or exactly or at most 85, at least or exactly or at most 86, at least or exactly or at most 87, at least or exactly or at most 88, at least or exactly or at most 89, at least or exactly or at most 90, at least or exactly or at most 91, at least or exactly or at most 92, at least or exactly or at most 93, at least or exactly or at most 94, at least or exactly or at most 95, at least or exactly or at most 96, at least or exactly or at most 97, at least or exactly or at most 98, at least or exactly or at most 99, at least or exactly or at most 100, at least or exactly or at most 101, at least or exactly or at most 102, at least or exactly or at most 103, at least or exactly or at most 104, at least or exactly or at most 105, at least or exactly or at most 106, at least or exactly or at most 107, at least or exactly or at most 108, at least or exactly or at most 109, at least or exactly or at most 110, at least or exactly or at most 111, at least or exactly or at most 112, at least or exactly or at most 113, at least or exactly or at most 114, at least or exactly or at most 115, at least or exactly or at most 116, at least or exactly or at most 117, at least or exactly or at most 118, at least or exactly or at most 119, at least or exactly or at most 120, at least or exactly or at most 121, at least or exactly or at most 122, at least or exactly or at most 123, at least or exactly or at most 124, at least or exactly or at most 125, at least or exactly or at most 126, at least or exactly or at most 127, at least or exactly or at most 128, at least or exactly or at most 129, at least or exactly or at most 130, at least or exactly or at most 131, at least or exactly or at most 132, at least or exactly or at most 133, at least or exactly or at most 134, at least or exactly or at most 135, at least or exactly or at most 136, at least or exactly or at most 137, at least or exactly or at most 138, at least or exactly or at most 139, at least or exactly or at most 140, at least or exactly or at most 141, at least or exactly or at most 142, at least or exactly or at most 143, at least or exactly or at most 144, at least or exactly or at most 145, at least or exactly or at most 146, at least or exactly or at most 147, at least or exactly or at most 148, at least or exactly or at most 149, at least or exactly or at most 150, at least or exactly or at most 151, at least or exactly or at most 152, at least or exactly or at most 153, at least or exactly or at most 154, at least or exactly or at most 155, at least or exactly or at most 156, at least or exactly or at most 157, at least or exactly or at most 158, at least or exactly or at most 159, at least or exactly or at most 160, at least or exactly or at most 171, at least or exactly or at most 172, at least or exactly or at most 173, at least or exactly or at most 174, at least or exactly or at most 175, at least or exactly or at most 176, at least or exactly or at most 177, at least or exactly or at most 178, at least or exactly or at most 179, at least or exactly or at most 180, at least or exactly or at most 181, at least or exactly or at most 182, at least or exactly or at most 183, at least or exactly or at most 184, at least or exactly or at most 185, at least or exactly or at most 186, at least or exactly or at most 187, at least or exactly or at most 188, at least or exactly or at most 189, at least or exactly or at most 190, at least or exactly or at most 191, at least or exactly or at most 192, at least or exactly or at most 193, at least or exactly or at most 194, at least or exactly or at most 195, at least or exactly or at most 196, at least or exactly or at most 197, at least or exactly or at most 198, at least or exactly or at most 199, at least or exactly or at most 200, at least or exactly or at most 201, at least or exactly or at most 202, at least or exactly or at most 203, at least or exactly or at most 204, at least or exactly or at most 205, at least or exactly or at most 206, at least or exactly or at most 207, at least or exactly or at most 208, at least or exactly or at most 209, at least or exactly or at most 210, at least or exactly or at most 211, at least or exactly or at most 212, at least or exactly or at most 213, at least or exactly or at most 214, at least or exactly or at most 215, at least or exactly or at most 216, at least or exactly or at most 217, at least or exactly or at most 218, at least or exactly or at most 219, at least or exactly or at most 220, at least or exactly or at most 221, at least or exactly or at most 222, at least or exactly or at most 223, at least or exactly or at most 224, at least or exactly or at most 225, at least or exactly or at most 226, at least or exactly or at most 227, at least or exactly or at most 228, at least or exactly or at most 229, at least or exactly or at most 230, at least or exactly or at most 231, at least or exactly or at most 232, at least or exactly or at most 233, at least or exactly or at most 234, at least or exactly or at most 235, at least or exactly or at most 236, at least or exactly or at most 237, at least or exactly or at most 238, at least or exactly or at most 239, at least or exactly or at most 240, at least or exactly or at most 241, at least or exactly or at most 242, at least or exactly or at most 243, at least or exactly or at most 244, at least or exactly or at most 245, at least or exactly or at most 246, at least or exactly or at most 247, at least or exactly or at most 248, at least or exactly or at most 249, at least or exactly or at most 250, at least or exactly or at most 251, at least or exactly or at most 252, at least or exactly or at most 253, at least or exactly or at most 254, at least or exactly or at most 255, at least or exactly or at most 256, at least or exactly or at most 257, at least or exactly or at most 258, at least or exactly or at most 259, at least or exactly or at most 260, at least or exactly or at most 271, at least or exactly or at most 272, at least or exactly or at most 273, at least or exactly or at most 274, at least or exactly or at most 275, at least or exactly or at most 276, at least or exactly or at most 277, at least or exactly or at most 278, at least or exactly or at most 279, at least or exactly or at most 280, at least or exactly or at most 281, at least or exactly or at most 282, at least or exactly or at most 283, at least or exactly or at most 284, at least or exactly or at most 285, at least or exactly or at most 286, at least or exactly or at most 287, at least or exactly or at most 288, at least or exactly or at most 289, at least or exactly or at most 290, at least or exactly or at most 291, at least or exactly or at most 292, at least or exactly or at most 293, at least or exactly or at most 294, at least or exactly or at most 295, at least or exactly or at most 296, at least or exactly or at most 297, at least or exactly or at most 298, at least or exactly or at most 299, at least or exactly or at most 300, at least or exactly or at most 301, at least or exactly or at most 302, at least or exactly or at most 303, at least or exactly or at most 304, at least or exactly or at most 305, at least or exactly or at most 306, at least or exactly or at most 307, at least or exactly or at most 308, at least or exactly or at most 309, at least or exactly or at most 310, at least or exactly or at most 311, at least or exactly or at most 312, at least or exactly or at most 313, at least or exactly or at most 314, at least or exactly or at most 315, at least or exactly or at most 316, at least or exactly or at most 317, at least or exactly or at most 318, at least or exactly or at most 319, at least or exactly or at most 320, at least or exactly or at most 321, at least or exactly or at most 322, at least or exactly or at most 323, at least or exactly or at most 324, at least or exactly or at most 325, at least or exactly or at most 326, at least or exactly or at most 327, at least or exactly or at most 328, at least or exactly or at most 329, at least or exactly or at most 330, at least or exactly or at most 331, at least or exactly or at most 332, at least or exactly or at most 333, at least or exactly or at most 334, at least or exactly or at most 335, at least or exactly or at most 336, at least or exactly or at most 337, at least or exactly or at most 338, at least or exactly or at most 339, at least or exactly or at most 340, at least or exactly or at most 341, at least or exactly or at most 342, at least or exactly or at most 343, at least or exactly or at most 344, at least or exactly or at most 345, at least or exactly or at most 346, at least or exactly or at most 347, at least or exactly or at most 348, at least or exactly or at most 349, at least or exactly or at most 350, at least or exactly or at most 351, at least or exactly or at most 352, at least or exactly or at most 353, at least or exactly or at most 354, at least or exactly or at most 355, at least or exactly or at most 356, at least or exactly or at most 357, at least or exactly or at most 358, at least or exactly or at most 359, at least or exactly or at most 360, at least or exactly or at most 361, at least or exactly or at most 362, at least or exactly or at most 363, at least or exactly or at most 364, at least or exactly or at most 365, at least or exactly or at most 366, at least or exactly or at most 367, at least or exactly or at most 368, at least or exactly or at most 369, at least or exactly or at most 370, at least or exactly or at most 371, at least or exactly or at most 372, at least or exactly or at most 373, at least or exactly or at most 374, at least or exactly or at most 375, at least or exactly or at most 376, at least or exactly or at most 377, at least or exactly or at most 378, at least or exactly or at most 379, at least or exactly or at most 380, at least or exactly or at most 381, at least or exactly or at most 382, at least or exactly or at most 383, at least or exactly or at most 384, at least or exactly or at most 385, at least or exactly or at most 386, at least or exactly or at most 387, at least or exactly or at most 388, at least or exactly or at most 389, at least or exactly or at most 390, at least or exactly or at most 391, at least or exactly or at most 392, at least or exactly or at most 393, at least or exactly or at most 394, at least or exactly or at most 395, at least or exactly or at most 396, at least or exactly or at most 397, at least or exactly or at most 398, at least or exactly or at most 399, at least or exactly or at most 400, at least or exactly or at most 401, at least or exactly or at most 402, at least or exactly or at most 403, at least or exactly or at most 404, at least or exactly or at most 405, at least or exactly or at most 406, at least or exactly or at most 407, at least or exactly or at most 408, at least or exactly or at most 409, at least or exactly or at most 410, at least or exactly or at most 411, at least or exactly or at most 412, at least or exactly or at most 413, at least or exactly or at most 414, at least or exactly or at most 415, at least or exactly or at most 416, at least or exactly or at most 417, at least or exactly or at most 418, at least or exactly or at most 419, at least or exactly or at most 420, at least or exactly or at most 421, at least or exactly or at most 422, and at least or exactly or at most 423 consecutive nucleotides in any one of SEQ ID NOs: 17-48, 50, and 51. Longer fragments are contemplated, i.e. fragments having at least or exactly or at most 200, at least or exactly or at most 300 at least or exactly or at most 400, at least or exactly or at most 500, at least or exactly or at most 600, at least or exactly or at most 700, at least or exactly or at most 800, at least or exactly or at most 900, at least or exactly or at most 1000, at least or exactly or at most 1500, at least or exactly or at most 2000, at least or exactly or at most 2500, at least or exactly or at most 3000, at least or exactly or at most 3500, or at least or exactly or at most 4000 nucleotides from those of SEQ ID NOs: 17-48, 50, and 51 that encompass fragments of such lengths.

The nucleic acid fragment of the invention discussed above typically has a sequence identity with the nucleotide sequence defined for i) or ii) above, which is at least 65%, such as at least 70%, at least 75%, at least 80%, at least 85%, at least 90%, at least 91%, at least 92%, at least 93%, at least 94%, at least 95%, at least 96%, at least 97%, at least 98%, and at least 99%.

The nucleic acid fragment of the invention discussed above may also have a sequence identity with the nucleotide sequence defined for iii) above, which is at least 65%, such as at least 70%, at least 75%, at least 80%, at least 85%, at least 90%, at least 91%, at least 92%, at least 93%, at least 94%, at least 95%, at least 96%, at least 97%, at least 98%, and at least 99%.

The nucleic acid fragment of the invention described above comprises in certain embodiments at least or exactly or at most X distinct nucleic acid sequences each encoding a polypeptide of the invention, where each of said X distinct nucleic acid sequences encodes at least or exactly or at most one immunogenic amino acid sequence present in or derived from any one of SEQ ID NOs: 1-16 and wherein said X distinct nucleic acid sequences together encode immunogenic amino acid sequences present in or derived from at least or exactly or at most X of SEQ ID NOs. 1-16, wherein X is an integer selected from 2, 3, 4, 5, 6, 7, 8, 9, 10, 11, 12, 13, 14, 15, and 16. In other words, such a nucleic acid fragment encodes several polypeptides of the invention. In some embodiments, the X nucleic acid sequences are expressed as separate encoded proteins and in other embodiments as “pearls on a string”, i.e. fused proteins. In some embodiments, immunogenic amino acid sequences from any one of SEQ ID NO: 16 are only present in one of said X nucleic acid sequences.

It will be understood that the nucleic acid fragments of the invention may be used for both production, carrier and vaccine purposes—the latter will require that the sequences are included in expression vectors that may lead to production of immunogenic proteins in the host animal receiving the vector.

### The Vectors of the Invention

Vectors of the invention fall into several categories discussed infra. One preferred vector of the invention comprises in operable linkage and in the 5′-3′ direction, an expression control region comprising an enhancer/promoter for driving expression of the nucleic acid fragment defined for option i) above, optionally a signal peptide coding sequence, a nucleotide sequence defined for option i), and optionally a terminator. Hence, such a vector constitutes an expression vector useful for effecting production in cells of the polypeptide of the invention. Since the polypeptides of the invention are bacterial of orgin, recombinant production is conveniently effected in bacterial host cells, so here it is preferred that the expression control region drives expression in prokaryotic cell such as a bacterium, e.g. in E coli. However, if the vector is to drive expression in mammalian cell (as would be the case for a DNA vaccine vector), the expression control region should be adapted to this particular use.

At any rate, certain vectors of the invention are capable of autonomous replication.

Also, the vector of the invention may be one that is capable of being integrated into the genome of a host cell—this is particularly useful if the vector is use in the production of stably transformed cells, where the progeny will also include the genetic information introduced via the vector. Alternatively, vectors incapable of being integrated into the genome of a mammalian host cell are useful in e.g. DNA vaccination.

Typically, the vector of the invention is selected from the group consisting of a virus, such as a attenuated virus (which may in itself be useful as a vaccine agent), a bacteriophage, a plasmid, a minichromosome, and a cosmid.

Particularly interesting vectors are viral vectors (in particular those useful as vaccine agents). These may be selected from the group consisting of a retrovirus vector, such as a lentivirus vector, an adenovirus vector, an adeno-associated virus vector, and a pox virus vector. Certain pox virus vectors are preferred, in particular vaccinia virus vectors. A particluarly preferred vaccinia virus vector is a modifed vaccinia Ankara (MVA) vector.

A more detailed discussion of vectors of the invention is provided in the following:

Polypeptides of the invention may be encoded by a nucleic acid molecule comprised in a vector. A nucleic acid sequence can be “heterologous,” which means that it is in a context foreign to the cell in which the vector is being introduced, which includes a sequence homologous to a sequence in the cell but in a position within the host cell where it is ordinarily not found. Vectors include naked DNAs, RNAs, plasmids, cosmids, viruses (bacteriophage, animal viruses, and plant viruses), and artificial chromosomes (e.g., YACs). One of skill in the art would be well equipped to construct a vector through standard recombinant techniques (for example Sambrook et al, 2001; Ausubel et al, 1996, both incorporated herein by reference). In addition to encoding the polypeptides of this invention, a vector of the present invention may encode polypeptide sequences such as a tag or immunogenicity enhancing peptide (e.g. an immunogenic carrier or a fusion partner that stimulates the immune system, such as a cytokine or active fragment thereof). Useful vectors encoding such fusion proteins include pIN vectors (Inouye et al, 1985), vectors encoding a stretch of histidines, and pGEX vectors, for use in generating glutathione S-transferase (GST) soluble fusion proteins for later purification and separation or cleavage.

Vectors of the invention may be used in a host cell to produce a polypeptide of the invention that may subsequently be purified for administration to a subject or the vector may be purified for direct administration to a subject for expression of the protein in the subject (as is the case when administering a nucleic acid vaccine).

Expression vectors can contain a variety of “control sequences,” which refer to nucleic acid sequences necessary for the transcription and possibly translation of an operably linked coding sequence in a particular host organism. In addition to control sequences that govern transcription and translation, vectors and expression vectors may contain nucleic acid sequences that serve other functions as well and are described infra.

### 1. Promoters and Enhancers

A “promoter” is a control sequence. The promoter is typically a region of a nucleic acid sequence at which initiation and rate of transcription are controlled. It may contain genetic elements at which regulatory proteins and molecules may bind such as RNA polymerase and other transcription factors. The phrases “operatively positioned,” “operatively linked,” “under control,” and “under transcriptional control” mean that a promoter is in a correct functional location and/or orientation in relation to a nucleic acid sequence to control transcriptional initiation and expression of that sequence. A promoter may or may not be used in conjunction with an “enhancer,” which refers to a cis-acting regulatory sequence involved in the transcriptional activation of a nucleic acid sequence.

A promoter may be one naturally associated with a gene or sequence, as may be obtained by isolating the 5′ non-coding sequences located upstream of the coding segment or exon. Such a promoter can be referred to as “endogenous.” Similarly, an enhancer may be one naturally associated with a nucleic acid sequence, located either downstream or upstream of that sequence. Alternatively, certain advantages will be gained by positioning the coding nucleic acid segment under the control of a recombinant or heterologous promoter, which refers to a promoter that is not normally associated with a nucleic acid sequence in its natural environment. A recombinant or heterologous enhancer refers also to an enhancer not normally associated with a nucleic acid sequence in its natural state. Such promoters or enhancers may include promoters or enhancers of other genes, and promoters or enhancers isolated from any other prokaryotic, viral, or eukaryotic cell, and promoters or enhancers not “naturally occurring,” i.e., containing different elements of different transcriptional regulatory regions, and/or mutations that alter expression. In addition to producing nucleic acid sequences of promoters and enhancers synthetically, sequences may be produced using recombinant cloning and/or nucleic acid amplification technology, including PCR™, in connection with the compositions disclosed herein (see U.S. Pat. Nos. 4,683,202, 5,928,906, each incorporated herein by reference).

Naturally, it may be important to employ a promoter and/or enhancer that effectively direct(s) the expression of the DNA segment in the cell type or organism chosen for expression. Those of skill in the art of molecular biology generally know the use of promoters, enhancers, and cell type combinations for protein expression (see Sambrook et al, 2001, incorporated herein by reference). The promoters employed may be constitutive, tissue-specific, or inducible and in certain embodiments may direct high level expression of the introduced DNA segment under specified conditions, such as large-scale production of recombinant proteins or peptides.

Examples of inducible elements, which are regions of a nucleic acid sequence that can be activated in response to a specific stimulus, include but are not limited to Immunoglobulin Heavy Chain (Banerji et al, 1983; Gilles et al, 1983; Grosschedl et al, 1985; Atchinson et al, 1986, 1987; toiler et al, 1987; Weinberger et al, 1984; Kiledjian et al, 1988; Porton et al; 1990), Immunoglobulin Light Chain (Queen et al, 1983; Picard et al, 1984), T Cell Receptor (Luria et al, 1987; Winoto et al, 1989; Redondo et al; 1990), HLA DQa and/or DQβ (Sullivan et al, 1987), (3-Interferon (Goodbourn et al, 1986; Fujita et al, 1987; Goodbourn et al, 1988), Interleukin-2 (Greene et al, 1989), Interleukin-2 Receptor (Greene et al, 1989; Lin et al, 1990), MHC Class II 5 (Koch et al, 1989), MHC Class II HLA-DRa (Sherman et al, 1989), β-Actin (Kawamoto et al, 1988; Ng et al; 1989), Muscle Creatine Kinase (MCK) (Jaynes et al, 1988; Horlick et al, 1989; Johnson et al, 1989), Prealbumin (Transthyretin) (Costa et al, 1988), Elastase I (Omitz et al, 1987), Metallothionein (MTII) (Karin et al, 1987; Culotta et al, 1989), Collagenase (Pinkert et al, 1987; Angel et al, 1987), Albumin (Pinkert et al, 1987; Tranche et al, 1989, 1990),α-Fetoprotein (Godbout et al, 1988; Campere et al, 1989), γ-Globin (Bodine et al, 1987; Perez-Stable et al, 1990),β-Globin (Trudel et al, 1987), c-fos (Cohen et al, 1987), c-HA-ras (Triesman, 1986; Deschamps et al, 1985), Insulin (Edlund et al, 1985), Neural Cell Adhesion Molecule (NCAM) (Hirsh et al, 1990),al-Antitrypain (Larimer et al, 1990), H2B (TH2B) Histone (Hwang et al, 1990), Mouse and/or Type I Collagen (Ripe et al, 1989), Glucose-Regulated Proteins (GRP94 and GRP78) (Chang et al, 1989), Rat Growth Hormone (Larsen et al, 1986), Human Serum Amyloid A (SAA) (Edbrooke et al, 1989), Troponin I (TN I) (Yutzey et al, 1989), Platelet-Derived Growth Factor (PDGF) (Pech et al, 1989), Duchenne Muscular Dystrophy (Klamut et al, 1990), SV40 (Banerji et al, 1981; Moreau et al, 1981 ; Sleigh et al, 1985; Firak et al, 1986; Herr et al, 1986; Imbra et al, 1986; Kadesch et al, 1986; Wang et al, 1986; Ondek et al, 1987; Kuhl et al, 1987; Schaffner et al, 1988), Polyoma (Swartzendruber et al, 1975; Vasseur et al, 1980; Katinka et al, 1980, 1981; Tyndell et al, 1981 ; Dandolo et al, 1983; de Villiers et al, 1984; Hen et al, 1986; Satake et al, 1988; Campbell et al, 1988), Retroviruses (Kriegler et al, 1982, 1983; Levinson et al, 1982; Kriegler et al, 1983, 1984a, b, 1988; Bosze et al, 1986; Miksicek et al, 1986; Celander et al, 1987; Thiesen et al, 1988; Celander et al, 1988; Choi et al, 1988; Reisman et al, 1989), Papilloma Virus (Campo et al, 1983; Lusky et al, 1983; Spandidos and Wilkie, 1983; Spalholz et al, 1985; Lusky et al, 1986; Cripe et al, 1987; Gloss et al, 1987; Hirochika et al, 1987; Stephens et al, 1987), Hepatitis B Virus (Bulla et al, 1986; Jameel et al, 1986; Shaul et al, 1987; Spandau et al, 1988; Vannice et al, 1988), Human Immunodeficiency Virus (Muesing et al, 1987; Hauber et al, 1988; Jakobovits et al, 1988; Feng et al, 1988; Takebe et al, 1988; Rosen et al, 1988; Berkhout et al, 1989; Laspia et al, 1989; Sharp et al, 1989; Braddock et al, 1989), Cytomegalovirus (CMV) IE (Weber et al, 1984; Boshart et al, 1985; Foecking et al, 1986), Gibbon Ape Leukemia Virus (Holbrook et al, 1987; Quinn et al, 1989).

Inducible Elements include, but are not limited to MT II—Phorbol Ester (TFA)/Heavy metals (Palmiter et al, 1982; Haslinger et al, 1985; Searle et al, 1985; Stuart et al, 1985; Imagawa et al, 1987, Karin et al, 1987; Angel et al, 1987b; McNeal) et al, 1989); MMTV (mouse mammary tumor virus)—Glucocorticoids (Huang et al, 1981; Lee et al, 1981; Majors et al, 1983; Chandler et al, 1983; Lee et al, 1984; Ponta et al, 1985; Sakai et al, 1988); β-Interferon —poly(rl)x/poly(rc) (Tavernier et al, 1983); Adenovirus 5 E2 -EIA (Imperiale et al, 1984); Collagenase—Phorbol Ester (TPA) (Angel et al, 1987a); Stromelysin—Phorbol Ester (TPA) (Angel et al, 1987b); SV40—Phorbol Ester (TPA) (Angel et al, 1987b); Murine MX Gene—Interferon, Newcastle Disease Virus (Hug et al, 1988); GRP78 Gene—A23187 (Resendez et al, 1988); α-2-Macroglobulin—IL-6 (Kunz et al, 1989); Vimentin—Serum (Rittling et al, 1989); MHC Class I Gene H-2κb—Interferon (Blanar et al, 1989); HSP70—E1A/SV40 Large T Antigen (Taylor et al, 1989, 1990a, 1990b); Proliferin—Phorbol Ester/TPA (Mordacq et al, 1989); Tumor Necrosis Factor—PMA (Hensel et al, 1989); and Thyroid Stimulating Hormonea Gene—Thyroid Hormone (Chatterjee et al, 1989).

Also contemplated as useful in the present invention are the dectin-1 and dectin-2 promoters. Additionally any promoter/enhancer combination (as per the Eukaryotic Promoter Data Base EPDB) could also be used to drive expression of structural genes encoding oligosaccharide processing enzymes, protein folding accessory proteins, selectable marker proteins or a heterologous protein of interest.

The particular promoter that is employed to control the expression of peptide or protein encoding polynucleotide of the invention is not believed to be critical, so long as it is capable of expressing the polynucleotide in a targeted cell, preferably a bacterial cell. Where a human cell is targeted, it is preferable to position the polynucleotide coding region adjacent to and under the control of a promoter that is capable of being expressed in a human cell. Generally speaking, such a promoter might include either a bacterial, human or viral promoter.

In various embodiments, the human cytomegalovirus (CMV) immediate early gene promoter, the SV40 early promoter, and the Rous sarcoma virus long terminal repeat can be used to obtain high level expression of a related polynucleotide to this invention. The use of other viral or mammalian cellular or bacterial phage promoters, which are well known in the art, to achieve expression of polynucleotides is contemplated as well.

In embodiments in which a vector is administered to a subject for expression of the protein, it is contemplated that a desirable promoter for use with the vector is one that is not down-regulated by cytokines or one that is strong enough that even if down-regulated, it produces an effective amount of the protein/polypeptide of the current invention in a subject to elicit an immune response. Non-limiting examples of these are CMV IE and RSV LTR. In other embodiments, a promoter that is up-regulated in the presence of cytokines is employed. The MHC I promoter increases expression in the presence of IFN-γ.

Tissue specific promoters can be used, particularly if expression is in cells in which expression of an antigen is desirable, such as dendritic cells or macrophages. The mammalian MHC I and MHC II promoters are examples of such tissue-specific promoters. 2. Initiation Signals and Internal Ribosome Binding Sites (IRES)

A specific initiation signal also may be required for efficient translation of coding sequences. These signals include the ATG initiation codon or adjacent sequences. Exogenous translational control signals, including the ATG initiation codon, may need to be provided. One of ordinary skill in the art would readily be capable of determining this and providing the necessary signals. It is well known that the initiation codon must be “in-frame” with the reading frame of the desired coding sequence to ensure translation of the entire insert. The exogenous translational control signals and initiation codons can be either natural or synthetic and may be operable in bacteria or mammalian cells. The efficiency of expression may be enhanced by the inclusion of appropriate transcription enhancer elements.

In certain embodiments of the invention, the use of internal ribosome entry sites (IRES) elements are used to create multigene, or polycistronic, messages. IRES elements are able to bypass the ribosome scanning model of 5′ methylated Cap dependent translation and begin translation at internal sites (Pelletier and Sonenberg, 1988). IRES elements from two members of the picornavirus family (polio and encephalomyocarditis) have been described (Pelletier and Sonenberg, 1988), as well an IRES from a mammalian message (Macejak and Sarnow, 1991). IRES elements can be linked to heterologous open reading frames. Multiple open reading frames can be transcribed together, each separated by an IRES, creating polycistronic messages. By virtue of the IRES element, each open reading frame is accessible to ribosomes for efficient translation. Multiple genes can be efficiently expressed using a single promoter/enhancer to transcribe a single message (see U.S. Pat. Nos. 5,925,565 and 5,935,819, herein incorporated by reference).

### 2. Multiple Cloning Sites

Vectors can include a multiple cloning site (MCS), which is a nucleic acid region that contains multiple restriction enzyme sites, any of which can be used in conjunction with standard recombinant technology to digest the vector. (See Carbonelli et al, 1999, Levenson et al, 1998, and Cocea, 1997, incorporated herein by reference.) Frequently, a vector is linearized or fragmented using a restriction enzyme that cuts within the MCS to enable exogenous sequences to be ligated to the vector. Techniques involving restriction enzymes and ligation reactions are well known to those of skill in the art of recombinant technology.

### 3. Splicing Sites

Most transcribed eukaryotic RNA molecules will undergo RNA splicing to remove introns from the primary transcripts. If relevant in the context of vectors of the present invention, vectors containing genomic eukaryotic sequences may require donor and/or acceptor splicing sites to ensure proper processing of the transcript for protein expression. (See Chandler et al, 1997, incorporated herein by reference.)

### 4. Termination Signals

The vectors or constructs of the present invention will generally comprise at least one termination signal. A “termination signal” or “terminator” is comprised of the DNA sequences involved in specific termination of an RNA transcript by an RNA polymerase. Thus, in certain embodiments a termination signal that ends the production of an RNA transcript is contemplated. A terminator may be necessary in vivo to achieve desirable message levels.

In eukaryotic systems, the terminator region may also comprise specific DNA sequences that permit site-specific cleavage of the new transcript so as to expose a polyadenylation site. This signals a specialized endogenous polymerase to add a stretch of about 200 A residues (poly A) to the 3′ end of the transcript. RNA molecules modified with this polyA tail appear to more stable and are translated more efficiently. Thus, in other embodiments involving eukaryotes, it is preferred that that terminator comprises a signal for the cleavage of the RNA, and it is more preferred that the terminator signal promotes polyadenylation of the message.

Terminators contemplated for use in the invention include any known terminator of transcription described herein or known to one of ordinary skill in the art, including but not limited to, for example, the bovine growth hormone terminator or viral termination sequences, such as the SV40 terminator. In certain embodiments, the termination signal may be a lack of transcribable or translatable sequence, such as due to a sequence truncation.

### 5. Polyadenylation Signals

In expression, particularly eukaryotic expression (as is relevant in nucleic acid vaccination), one will typically include a polyadenylation signal to effect proper polyadenylation of the transcript. The nature of the polyadenylation signal is not believed to be crucial to the successful practice of the invention, and/or any such sequence may be employed. Preferred embodiments include the SV40 polyadenylation signal and/or the bovine growth hormone polyadenylation signal, convenient and/or known to function well in various target cells. Polyadenylation may increase the stability of the transcript or may facilitate cytoplasmic transport.

### 6. Origins of Replication

In order to propagate a vector in a host cell, it may contain one or more origins of replication sites (often termed “on”), which is a specific nucleic acid sequence at which replication is initiated. Alternatively an autonomously replicating sequence (ARS) can be employed if the host cell is yeast.

### 7. Selectable and Screenable Markers

In certain embodiments of the invention, cells containing a nucleic acid construct of the present invention may be identified in vitro or in vivo by encoding a screenable or selectable marker in the expression vector. When transcribed and translated, a marker confers an identifiable change to the cell permitting easy identification of cells containing the expression vector. Generally, a selectable marker is one that confers a property that allows for selection. A positive selectable marker is one in which the presence of the marker allows for its selection, while a negative selectable marker is one in which its presence prevents its selection. An example of a positive selectable marker is a drug resistance marker.

Usually the inclusion of a drug selection marker aids in the cloning and identification of transformants, for example, markers that confer resistance to neomycin, puromycin, hygromycin, DHFR, GPT, zeocin or histidinol are useful selectable markers. In addition to markers conferring a phenotype that allows for the discrimination of transformants based on the implementation of conditions, other types of markers including screenable markers such as GFP for colorimetric analysis. Alternatively, screenable enzymes such as herpes simplex virus thymidine kinase (tk) or chloramphenicol acetyltransferase (CAT) may be utilized. One of skill in the art would also know how to employ immunologic markers that can be used in conjunction with FACS analysis. The marker used is not believed to be important, so long as it is capable of being expressed simultaneously with the nucleic acid encoding a protein of the invention. Further examples of selectable and screenable markers are well known to one of skill in the art.

### The Transformed Cells of the Invention

Transformed cells of the invention are useful as organisms for producing the polypeptide of the invention, but also as simple “containers” of nucleic acids and vectors of the invention.

Certain transformed cells of the invention are capable of replicating the nucleic acid fragment defined for option i) of the second aspect of the invention. Preferred transformed cells of the invention are capable of expressing the nucleic acid fragment defined for option i).

For recombinant production it is convenient, but not a prerequisite that the transformed cell according is prokaryotic, such as a bacterium, but generally both prokaryotic cells and eukaryotic cells may be used.

Suitable prokaryotic cells are bacterial cells selected from the group consisting of Escherichia (such as E. coli.), Bacillus [e.g. Bacillus subtilis], Salmonella, and Mycobacterium [preferably non-pathogenic, e.g. M. bovis BCG].

Eukaryotic cells can be in the form of yeasts (such as Saccharomyces cerevisiae) and protozoans. Alternatively, the transformed eukaryotic cells are derived from a multicellular organism such as a fungus, an insect cell, a plant cell, or a mammalian cell.

For production purposes, it is advantageous that the transformed cell of the invention is is stably transformed by having the nucleic acid defined above for option i) stably integrated into its genome, and in certain embodiments it is also preferred that the transformed cell secretes or carries on its surface the polypeptide of the invention, since this facilitates recovery of the polypeptides produced. A particular version of this embodiment is one where the transformed cell is a bacterium and secretion of the polypeptide of the invention is into the periplasmic space.

As noted above, stably transformed cells are preferred—these i.a. allows that cell lines comprised of transformed cells as defined herein may be established—such cell lines are partilucarly preferred aspects of the invention.

Further details on cells and cell lines are presented in the following:

Suitable cells for recombinant nucleic acid expression of the nucleic acid fragments of the present invention are prokaryotes and eukaryotes. Examples of prokaryotic cells include E. coli; members of the Staphylococcus genus, such as S. epidermidis; members of the Lactobacillus genus, such as L. plantarum; members of the Lactococcus genus, such as L. lactis; members of the Bacillus genus, such as B. subtilis; members of the Corynebacterium genus such as C. glutamicum; and members of the Pseudomonas genus such as Ps. fluorescens. Examples of eukaryotic cells include mammalian cells; insect cells; yeast cells such as members of the Saccharomyces genus (e.g. S. cerevisiae), members of the Pichia genus (e.g. P. pastoris), members of the Hansenula genus (e.g. H. polymorpha), members of the Kluyveromyces genus (e.g. K. lactis or K. fragilis) and members of the Schizosaccharomyces genus (e.g. S. pombe).

Techniques for recombinant gene production, introduction into a cell, and recombinant gene expression are well known in the art. Examples of such techniques are provided in references such as Ausubel, Current Protocols in Molecular Biology, John Wiley, 1987-2002, and Sambrook et al., Molecular Cloning, A Laboratory Manual, 2 nd Edition, Cold Spring Harbor Laboratory Press, 1989.

As used herein, the terms “cell,” “cell line,” and “cell culture” may be used interchangeably. All of these terms also include their progeny, which is any and all subsequent generations. It is understood that all progeny may not be identical due to deliberate or inadvertent mutations. In the context of expressing a heterologous nucleic acid sequence, “host cell” refers to a prokaryotic or eukaryotic cell, and it includes any transformable organism that is capable of replicating a vector or expressing a heterologous gene encoded by a vector. A host cell can, and has been, used as a recipient for vectors or viruses. A host cell may be “transfected” or “transformed,” which refers to a process by which exogenous nucleic acid, such as a recombinant protein-encoding sequence, is transferred or introduced into the host cell. A transformed cell includes the primary subject cell and its progeny.

Host cells may be derived from prokaryotes or eukaryotes, including bacteria, yeast cells, insect cells, and mammalian cells for replication of the vector or expression of part or all of the nucleic acid sequence(s). Numerous cell lines and cultures are available for use as a host cell, and they can be obtained through the American Type Culture Collection (ATCC), which is an organization that serves as an archive for living cultures and genetic materials (www.atcc.org) or from other depository institutions such as Deutsche Sammlung vor Micrroorganismen and Zellkulturen (DSM). An appropriate host can be determined by one of skill in the art based on the vector backbone and the desired result. A plasmid or cosmid, for example, can be introduced into a prokaryote host cell for replication of many vectors or expression of encoded proteins. Bacterial cells used as host cells for vector replication and/or expression include Staphylococcus strains, DH5α, JMI 09, and KC8, as well as a number of commercially available bacterial hosts such as SURE(R) Competent Cells and SOLOP ACK(™) Gold Cells (STRATAGENE®, La Jolla, CA). Alternatively, bacterial cells such as E. coli LE392 could be used as host cells for phage viruses. Appropriate yeast cells include Saccharomyces cerevisiae, Saccharomyces pombe, and Pichia pastoris.

Examples of eukaryotic host cells for replication and/or expression of a vector include HeLa, NIH3T3, Jurkat, 293, Cos, CHO, Saos, and PC12. Many host cells from various cell types and organisms are available and would be known to one of skill in the art. Similarly, a viral vector may be used in conjunction with either a eukaryotic or prokaryotic host cell, particularly one that is permissive for replication or expression of the vector.

Some vectors may employ control sequences that allow it to be replicated and/or expressed in both prokaryotic and eukaryotic cells. One of skill in the art would further understand the conditions under which to incubate all of the above described host cells to maintain them and to permit replication of a vector. Also understood and known are techniques and conditions that would allow large-scale production of vectors, as well as production of the nucleic acids encoded by vectors and their cognate polypeptides, proteins, or peptides.

### Expression Systems

Numerous expression systems exist that comprise at least a part or all of the compositions discussed above. Prokaryote- and/or eukaryote-based systems can be employed for use with the present invention to produce nucleic acid sequences, or their cognate polypeptides, proteins and peptides. Many such systems are commercially and widely available.

The insect cell/baculovirus system can produce a high level of protein expression of a heterologous nucleic acid segment, such as described in U.S. Pat. Nos. 5,871,986, 4,879,236, both herein incorporated by reference, and which can be bought, for example, under the name MAXBAC® 2.0 from INVITROGEN® and BACPACK™ Baculovirus expression system from CLONTECH®

In addition to the disclosed expression systems of the invention, other examples of expression systems include STRATAGENEφ's COMPLETE CONTROL™ Inducible Mammalian Expression System, which involves a synthetic ecdysone-inducible receptor, or its pET Expression System, an E. coli expression system. Another example of an inducible expression system is available from INVITROGEN®, which carries the T-REX™ (tetracycline-regulated expression) System, an inducible mammalian expression system that uses the full-length CMV promoter. INVITROGEN® also provides a yeast expression system called the Pichia methanolica Expression System, which is designed for high-level production of recombinant proteins in the methylotrophic yeast Pichia methanolica. One of skill in the art would know how to express a vector, such as an expression construct, to produce a nucleic acid sequence or its cognate polypeptide, protein, or peptide.

### Amplification of Nucleic Acids

Nucleic acids used as a template for amplification may be isolated from cells, tissues or other samples according to standard methodologies (Sambrook et al, 2001). In certain embodiments, analysis is performed on whole cell or tissue homogenates or biological fluid samples without substantial purification of the template nucleic acid. The nucleic acid may be genomic DNA or fractionated or whole cell RNA. Where RNA is used, it may be desired to first convert the RNA to a complementary DNA.

The term “primer,” as used herein, is meant to encompass any nucleic acid that is capable of priming the synthesis of a nascent nucleic acid in a template-dependent process. Typically, primers are oligonucleotides from ten to twenty and/or thirty base pairs in length, but longer sequences can be employed. Primers may be provided in double-stranded and/or single-stranded form, although the single-stranded form is preferred.

Pairs of primers designed to selectively hybridize to nucleic acids corresponding to sequences of genes identified herein are contacted with the template nucleic acid under conditions that permit selective hybridization. Depending upon the desired application, high stringency hybridization conditions may be selected that will only allow hybridization to sequences that are completely complementary to the primers. In other embodiments, hybridization may occur under reduced stringency to allow for amplification of nucleic acids containing one or more mismatches with the primer sequences. Once hybridized, the template-primer complex is contacted with one or more enzymes that facilitate template-dependent nucleic acid synthesis. Multiple rounds of amplification, also referred to as “cycles,” are conducted until a sufficient amount of amplification product is produced.

The amplification product may be detected or quantified. In certain applications, the detection may be performed by visual means. Alternatively, the detection may involve indirect identification of the product via chemiluminescence, radioactive scintigraphy of incorporated radiolabel or fluorescent label or even via a system using electrical and/or thermal impulse signals (Bellus, 1994).

A number of template dependent processes are available to amplify the oligonucleotide sequences present in a given template sample. One of the best known amplification methods is the polymerase chain reaction (referred to as PCR(™)) which is described in detail in U.S. Pat. Nos. 4,683,195, 4,683,202 and 4,800,159, and in Innis et al., 1988, each of which is incorporated herein by reference in their entirety.

Alternative methods for amplification of target nucleic acid sequences that may be used in the practice of the present invention are disclosed in U.S. Pat. Nos. 5,843,650, 5,846,709, 5,846,783, 5,849,546, 5,849,497, 5,849,547, 5,858,652, 5,866,366, 5,916,776, 5,922,574, 5,928,905, 5,928,906, 5,932,451, 5,935,825, 5,939,291 and 5,942,391, GB Application No. 2 202 328, and in PCT Application No. PCT/US89/01025, each of which is incorporated herein by reference in its entirety.

### Methods of Gene Transfer

Suitable methods for nucleic acid delivery to effect expression of compositions of the present invention are believed to include virtually any method by which a nucleic acid (e.g., DNA, including viral and nonviral vectors) can be introduced into a cell, a tissue or an organism, as described herein or as would be known to one of ordinary skill in the art. Such methods include, but are not limited to, direct delivery of DNA such as by injection (U.S. Pat. Nos. 5,994,624, 5,981,274, 5,945,100, 5,780,448, 5,736,524, 5,702,932, 5,656,610, 5,589,466 and 5,580,859, each incorporated herein by reference), including microinjection (Harland and Weintraub, 1985; U.S. Pat. Nos. 5,789,215, incorporated herein by reference); by electroporation (U.S. Pat. No. 5,384,253, incorporated herein by reference); by calcium phosphate precipitation (Graham and Van Der Eb, 1973; Chen and Okayama, 1987; Rippe et al., 1990); by using DEAE dextran followed by polyethylene glycol (Gopal, 1985); by direct sonic loading (Fechheimer et al, 1987); by liposome mediated transfection (Nicolau and Sene, 1982; Fraley et al, 1979; Nicolau et al, 1987; Wong et al, 1980; Kaneda et al, 1989; Kato et al, 1991); by microprojectile bombardment (PCT Application Nos. WO 94/09699 and 95/06128; U.S. Pat. Nos. 5,610,042; 5,322,783 5,563,055, 5,550,318, 5,538,877 and 5,538,880, and each incorporated herein by reference); by agitation with silicon carbide fibers (Kaeppler et al, 1990; U.S. Pat. Nos. 5,302,523 and 5,464,765, each incorporated herein by reference); by Agrobacterium mediated transformation (U.S. Pat. Nos. 5,591,616 and 5,563,055, each incorporated herein by reference); or by PEG mediated transformation of protoplasts (Omirulleh et al, 1993; U.S. Pat. Nos. 4,684,611 and 4,952,500, each incorporated herein by reference); by desiccation/inhibition mediated DNA uptake (Potrykus et al, 1985). Through the application of techniques such as these, organelle(s), cell(s), tissue(s) or organism(s) may be stably or transiently transformed.

### The Antibodies of the Invention—and Their Production/Isolation

Antibodies directed against the proteins of the invention are useful for affinity chromatography, immunoassays, and for distinguishing/identifying staphylococcus proteins as well as for passive immunisation and therapy.

Antibodies to the proteins of the invention, both polyclonal and monoclonal, may be prepared by conventional methods. In general, the protein is first used to immunize a suitable animal, preferably a mouse, rat, rabbit or goat. Rabbits and goats are preferred for the preparation of polyclonal sera due to the volume of serum obtainable, and the availability of labeled anti-rabbit and anti-goat antibodies. Immunization is generally performed by mixing or emulsifying the protein in saline, preferably in an adjuvant such as Freund's complete adjuvant, and injecting the mixture or emulsion parenterally (generally subcutaneously or intramuscularly). A dose of 50-200 μg/injection is typically sufficient. Immunization is generally boosted 2-6 weeks later with one or more injections of the protein in saline, preferably using Freund's incomplete adjuvant. One may alternatively generate antibodies by in vitro immunization using methods known in the art, which for the purposes of this invention is considered equivalent to in vivo immunization. Polyclonal antiserum is obtained by bleeding the immunized animal into a glass or plastic container, incubating the blood at 25 C for one hour, followed by incubating at 4 C for 2-18 hours. The serum is recovered by centrifugation (eg. 1,000 g for minutes). About 20-50 ml per bleed may be obtained from rabbits.

Monoclonal antibodies are prepared using the standard method of Kohler & Milstein [Nature (1975) 256 : 495-96], or a modification thereof. Typically, a mouse or rat is immunized as described above. However, rather than bleeding the animal to extract serum, the spleen (and optionally several large lymph nodes) is removed and dissociated into single cells. If desired, the spleen cells may be screened (after removal of nonspecifically adherent cells) by applying a cell suspension to a plate or well coated with the protein antigen. B-cells expressing membrane-bound immunoglobulin specific for the antigen bind to the plate, and are not rinsed away with the rest of the suspension. Resulting B-cells, or all dissociated spleen cells, are then induced to fuse with myeloma cells to form hybridomas, and are cultured in a selective I aedium (elg. hypexanthine, aminopterin, thymidine medium, “HAT”). The resulting hybridomas are plated by limiting dilution, and are assayed for production of antibodies, which bind specifically to the immunizing antigen (and which do not bind to unrelated antigens). The selected MAb-secreting hybridomas are then cultured either in vitro (eg. in tissue culture bottles or hollow fiber reactors), or in vivo (as ascites in mice).

If desired, the antibodies (whether polyclonal or monoclonal) may be labeled using conventional techniques. Suitable labels include fluorophores, chromophores, radioactive atoms (particularly 32p and 125I), electron-dense reagents, enzymes, and ligands having specific binding partners. Enzymes are typically detected by their activity. For example, horseradish peroxidase is usually detected by its ability to convert 3,3′, 5,5′-tetramethylbenzidine (TMB) to a blue pigment, quantifiable with a spectrophotometer. “Specific binding partner” refers to a protein capable of binding a ligand molecule with high specificity, as for example in the case of an antigen and a monoclonal antibody specific therefor. Other specific binding partners include biotin and avidin or streptavidin, IgG and protein A, and the numerous receptor-ligand couples known in the art. It should be understood that the above description is not meant to categorize the various labels into distinct classes, as the same label may serve in several different modes. For example, 1151 may serve as a radioactive label or as an electron-dense reagent. HRP may serve as enzyme or as antigen for a MAb. Further, one may combine various labels for desired effect. For example, MAbs and avidin also require labels in the practice of this invention: thus, one might label a MAb with biotin, and detect its presence with avidin labeled with, 125I, or with an anti-biotin MAb labeled with HRP. Other permutations and possibilities will be readily apparent to those of ordinary skill in the art, and are considered as equivalents within the scope of the instant invention.

According to the invention, the isolated monoclonal antibody or antibody analogue is preferably a monoclonal antibody selected from a multi-domain antibody such as a murine antibody, a chimeric antibody such as a humanized antibody, a fully human antibody, and single-domain antibody of a llama or a camel, or which is an antibody analogue selected from a fragment of an antibody such as an Fab or an F(ab′)₂, an scFV; cf. also the definition of the term “antibody” presented above.

### Compositions of the Invention; Vaccines

Pharmaceutical compositions, in particular vaccines, according to the invention may either be prophylactic (ie. to prevent infection) or therapeutic (ie, to treat disease after infection).

Such vaccines comprise immunising antigen(s), immunogen(s), polypeptide(s), protein(s) or nucleic acid(s), usually in combination with “pharmaceutically acceptable carriers”, which include any carrier that does not itself induce the production of antibodies harmful to the individual receiving the composition.

In some embodiments of the invention, the pharmaceutical compositions such as vaccines include merely one single antigen, immunogen, polypeptide, protein, nucleic acid or vector of the invention, but in other embodiments, the pharmaceutical compositions comprise “cocktails” of the antigens or of the immunogens or of the polypeptides or of the protein or of the nucleic acids or of the vectors of the invention.

In particularly interesting embodiments, the pharmaceutical composition is an MVA vector mentioned herein, which encodes and can effect expression of at least 2 nucleic acid fragments of the invention.

Another interesting embodiment of a pharmaceutical composition comprises RNA as the active principle, i.e. at least one mRNA encoding a polypeptide of the invention.

An embodiment of a pharmaceutical composition of the invention comprises Y or at least Y or at most Y distinct polypeptides of the invention described above, where each of said Y or at least Y or at most Y distinct polypeptides comprises an immunogenic amino acid sequence present in or derived from any one of SEQ ID NOs: 1-16 and wherein said Y or at least Y or at most Y distinct polypeptides together comprise immunogenic amino acid sequences present in or derived from Y or at least Y or at most Y of SEQ ID NOs. 1-16, wherein Y is an integer selected from 2, 3, 4, 5, 6, 7, 8, 9, 10, 11, 12, 13, 14, 15, and 16.

Another embodiment of the pharmaceutical composition of the invention comprises Z or at least Z or at most Z distinct nucleic acid molecules (such as DNA and RNA) each encoding a polypeptide of the invention, where each of said Z or at least Z or at most Z distinct nucleic acid molecules encodes an immunogenic amino acid sequence present in or derived from any one of SEQ ID NOs: 1-16 and wherein said at Z or least Z distinct nucleic acid molecules together encode immunogenic amino acid sequences present in or derived from Z or at least Z or at most Z of SEQ ID NOs. 1-16, wherein Z is an integer selected from 2, 3, 4, 5, 6, 7, 8, 9, 10, 11, 12, 13, 14, 15, and 16.

Suitable carriers are typically large, slowly metabolized macromolecules such as proteins, polysaccharides, polylactic acids, polyglycolic acids, polymeric amino acids, amino acid copolymers, lipid aggregates (such as oil droplets or liposomes), and inactive virus particles.

Such carriers are well known to those of ordinary skill in the art. Additionally, these carriers may function as immunostimulating agents (“adjuvants”). Furthermore, the antigen or immunogen may be conjugated to a bacterial toxoid, such as a toxoid from diphtheria, tetanus, cholera, H. pylori, etc. pathogen, cf. the description of immunogenic carriers supra.

The pharmaceutical compositions of the invention thus typically contain an immunological adjuvant, which is commonly an aluminium based adjuvant or one of the other adjuvants described in the following:

Preferred adjuvants to enhance effectiveness of the composition include, but are not limited to: (1) aluminum salts (alum), such as aluminum hydroxide, aluminum phosphate, aluminum sulfate, etc; (2) oil-in-water emulsion formulations (with or without other specific immunostimulating agents such as muramyl peptides (see below) or bacterial cell wall components), such as for example (a) MF59 (WO 90/14837; Chapter 10 in Vaccine design: the subunit and adjuvant approach, eds. Powell & Newman, Plenum Press 1995), containing 5% Squalene, 0.5% Tween 80, and 0.5% Span 85 (optionally containing various amounts of MTP-PE (see below), although not required) formulated into submicron particles using a microfluidizer such as Model 110Y microfluidizer (Microfluidics, Newton, MA), (b) SAF, containing 10% Squalane, 0.4% Tween 80, 5% pluronic-blocked polymer L121, and thr-MDP see below) either microfluidized into a submicron emulsion or vortexed to generate a larger particle size emulsion, and (c) Ribi adjuvant system (RAS), (Ribi Immunochem, Hamilton, MT) containing 2% Squalene, 0.2% Tween 80, and one or more bacterial cell wall components from the group consisting of monophosphoryl lipid A (MPL), trehalose dimycolate (TDM), and cell wall skeleton (CWS), preferably MPL+CWS (Detox™); (3) saponin adjuvants such as Stimulon™ (Cambridge Bioscience, Worcester, MA) may be used or particles generated therefrom such as ISCOMs (immunostimulating complexes); (4) Complete Freund's Adjuvant (CFA) and Incomplete Freund's Adjuvant (IFA); (5) cytokines, such as interleukins (eg. IL-1, IL-2, IL-4, IL-5, IL-6, IL-7, IL-12, etc.), interferons (eg. gamma interferon), macrophage colony stimulating factor (M-CSF), tumor necrosis factor (TNF), etc.; and (6) other substances that act as immunostimulating agents to enhance the effectiveness of the composition. Alum and MF59™ adjuvants are preferred.

As mentioned above, muramyl peptides include, but are not limited to, N-acetyl-muramyl-L-threonyl-D-isoglutamine (thr-MDP), N-acetyl-normuramyl-L-alanyl-D-isoglutamine (nor-MDP), N-acetylmuramyl-L-alanyl-D-isoglutaminyl-L-alanine-2″-2′-dipalmitoyl-sn-glycero-3-hydroxyphosphoryloxy)-ethylamine (MTP-PE), etc.

The immunogenic compositions (eg. the immunising antigen or immunogen or polypeptide or protein or nucleic acid, pharmaceutically acceptable carrier, and adjuvant) typically will contain diluents, such as water, saline, glycerol, ethanol, etc. Additionally, auxiliary substances, such as wetting or emulsifying agents, pH buffering substances, and the like, may be present in such vehicles.

Typically, the immunogenic compositions are prepared as injectables, either as liquid solutions or suspensions; solid forms suitable for solution in, or suspension in, liquid vehicles prior to injection may also be prepared. The preparation also may be emulsified or encapsulated in liposomes for enhanced adjuvant effect, as discussed above under pharmaceutically acceptable carriers.

Immunogenic compositions used as vaccines comprise an immunologically effective amount of the antigenic or immunogenic polypeptides, as well as any other of the above-mentioned components, as needed. By “immunollogically effective amount”, it is meant that the administration of that amount to an individual, either in a single dose or as part of a series, is effective for treatment or prevention. This amount varies depending upon the health and physical condition of the individual to be treated, the taxonomic group of individual to be treated (eg. nonhuma primate, primate, etc.), the capacity of the individual's immune system to synthesize antibodies or generally mount an immune response, the degree of protection desired, the formulation of the vaccine, the treating doctor's assessment of the medical situation, and other relevant factors. It is expected that the amount will fall in a relatively broad range that can be determined through routine trials. However, for the purposes of protein vaccination, the amount administered per immunization is typically in the range between 0.5 μg and 500 mg (however, often not higher than 5,000 μg), and very often in the range between 10 and 200 μg.

The immunogenic compositions are conventionally administered parenterally, eg, by injection, either subcutaneously, intramuscularly, or transdermally/transcutaneously (eg. W098/20734). Additional formulations suitable for other modes of administration include oral and pulmonary formulations, suppositories, and transdermal applications. In the case of nucleic acid vaccination, also the intravenous or intraarterial routes may be applicable.

Dosage treatment may be a single dose schedule or a multiple dose schedule. The vaccine may be administered in conjunction with other immunoregulatory agents.

As an alternative to protein-based vaccines, DNA vaccination (also termed nucleic acid vaccination or gene vaccination) may be used [eg. Robinson & Torres (1997) Seminars in Immunol 9: 271-283; Donnelly et al. (1997) Annu Rev Immunol 15: 617-648; later herein].

A further aspect of the invention is as mentioned above the recognition that combination vaccines can be provided, wherein 2 or more antigens disclosed herein are combined to enhance the immune response by the vaccinated animal, including to optimize initial immune response and duration of immunity. For the purposes of this aspect of the invention, multiple antigenic fragments derived from the same, longer protein can also be used, such as the use of a combination of different lengths of polypeptide sequence fragments from one protein.

Thus, embodiments of the invention relate to a composition (or the use as a vaccine thereof) comprising 2 distinct (i.e. non-identical) proteinaceaous immunogens disclosed herein wherein the first of said immunogens is SEQ ID NO: 1 or a variant or fragment thereof disclosed herein in combination with a proteinaceous immunogen selected from any one of SEQ ID NOs: 1, 2, 3, 4, 5, 6, 7, 8, 9, 10, 11, 12, 13, 14, 15, and 16 or in combination with a variant or fragment disclosed herein of any one of SEQ ID NOs: 1, 2, 3, 4, 5, 6, 7, 8, 9, 10, 11, 12, 13, 14, 15, and 16.

Also, embodiments of the invention relate to a composition (or the use as a vaccine thereof) comprising 2 distinct (i.e. non-identical) proteinaceaous immunogens disclosed herein wherein the first of said immunogens is SEQ ID NO: 2 or a variant or fragment thereof disclosed herein in combination with a proteinaceous immunogen selected from any one of

SEQ ID NOs: 1, 3, 3, 4, 5, 6, 7, 8, 9, 10, 11, 12, 13, 14, 15, and 16 or in combination with a variant or fragment disclosed herein of any one of SEQ ID NOs: 1, 2, 3, 4, 5, 6, 7, 8, 9, 10, 11, 12, 13, 14, 15, and 16.

Also, embodiments of the invention relate to a composition (or the use as a vaccine thereof) comprising 2 distinct (i.e. non-identical) proteinaceaous immunogens disclosed herein wherein the first of said immunogens is SEQ ID NO: 3 or a variant or fragment thereof disclosed herein in combination with a proteinaceous immunogen selected from any one of SEQ ID NOs: 1, 2, 3, 4, 5, 6, 7, 8, 9, 10, 11, 12, 13, 14, 15, and 16 or in combination with a variant or fragment disclosed herein of any one of SEQ ID NOs: 1, 2, 3, 4, 5, 6, 7, 8, 9, 10, 11, 12, 13, 14, 15, and 16.

Also, embodiments of the invention relate to a composition (or the use as a vaccine thereof) comprising 2 distinct (i.e. non-identical) proteinaceaous immunogens disclosed herein wherein the first of said immunogens is SEQ ID NO: 4 or a variant or fragment thereof disclosed herein in combination with a proteinaceous immunogen selected from any one of SEQ ID NOs: 1, 2, 3, 4, 5, 6, 7, 8, 9, 10, 11, 12, 13, 14, 15, and 16 or in combination with a variant or fragment disclosed herein of any one of SEQ ID NOs: 1, 2, 3, 4, 5, 6, 7, 8, 9, 10, 11, 12, 13, 14, 15, and 16.

Also, embodiments of the invention relate to a composition (or the use as a vaccine thereof) comprising 2 distinct (i.e. non-identical) proteinaceaous immunogens disclosed herein wherein the first of said immunogens is SEQ ID NO: 5 or a variant or fragment thereof disclosed herein in combination with a proteinaceous immunogen selected from any one of SEQ ID NOs: 1, 2, 3, 4, 5, 6, 7, 8, 9, 10, 11, 12, 13, 14, 15, and 16 or in combination with a variant or fragment disclosed herein of any one of SEQ ID NOs: 1, 2, 3, 4, 5, 6, 7, 8, 9, 10, 11, 12, 13, 14, 15, and 16.

Also, embodiments of the invention relate to a composition (or the use as a vaccine thereof) comprising 2 distinct (i.e. non-identical) proteinaceaous immunogens disclosed herein wherein the first of said immunogens is SEQ ID NO: 6 or a variant or fragment thereof disclosed herein in combination with a proteinaceous immunogen selected from any one of SEQ ID NOs: 1, 2, 3, 4, 5, 6, 7, 8, 9, 10, 11, 12, 13, 14, 15, and 16 or in combination with a variant or fragment disclosed herein of any one of SEQ ID NOs: 1, 2, 3, 4, 5, 6, 7, 8, 9, 10, 11, 12, 13, 14, 15, and 16.

Also, embodiments of the invention relate to a composition (or the use as a vaccine thereof) comprising 2 distinct (i.e. non-identical) proteinaceaous immunogens disclosed herein wherein the first of said immunogens is SEQ ID NO: 7 or a variant or fragment thereof disclosed herein in combination with a proteinaceous immunogen selected from any one of SEQ ID NOs: 1, 2, 3, 4, 5, 6, 7, 8, 9, 10, 11, 12, 13, 14, 15, and 16 or in combination with a variant or fragment disclosed herein of any one of SEQ ID NOs: 1, 2, 3, 4, 5, 6, 7, 8, 9, 10, 11, 12, 13, 14, 15, and 16.

Also, embodiments of the invention relate to a composition (or the use as a vaccine thereof) comprising 2 distinct (i.e. non-identical) proteinaceaous immunogens disclosed herein wherein the first of said immunogens is SEQ ID NO: 8 or a variant or fragment thereof disclosed herein in combination with a proteinaceous immunogen selected from any one of SEQ ID NOs: 1, 2, 3, 4, 5, 6, 7, 8, 9, 10, 11, 12, 13, 14, 15, and 16 or in combination with a variant or fragment disclosed herein of any one of SEQ ID NOs: 1, 2, 3, 4, 5, 6, 7, 8, 9, 10, 11, 12, 13, 14, 15, and 16.

Also, embodiments of the invention relate to a composition (or the use as a vaccine thereof) comprising 2 distinct (i.e. non-identical) proteinaceaous immunogens disclosed herein wherein the first of said immunogens is SEQ ID NO: 9 or a variant or fragment thereof disclosed herein in combination with a proteinaceous immunogen selected from any one of SEQ ID NOs: 1, 2, 3, 4, 5, 6, 7, 8, 9, 10, 11, 12, 13, 14, 15, and 16 or in combination with a variant or fragment disclosed herein of any one of SEQ ID NOs: 1, 2, 3, 4, 5, 6, 7, 8, 9, 10, 11, 12, 13, 14, 15, and 16.

Also, embodiments of the invention relate to a composition (or the use as a vaccine thereof) comprising 2 distinct (i.e. non-identical) proteinaceaous immunogens disclosed herein wherein the first of said immunogens is SEQ ID NO: 10 or a variant or fragment thereof disclosed herein in combination with a proteinaceous immunogen selected from any one of SEQ ID NOs: 1, 2, 3, 4, 5, 6, 7, 8, 9, 10, 11, 12, 13, 14, 15, and 16 or in combination with a variant or fragment disclosed herein of any one of SEQ ID NOs: 1, 2, 3, 4, 5, 6, 7, 8, 9, 10, 11, 12, 13, 14, 15, and 16.

Also, embodiments of the invention relate to a composition (or the use as a vaccine thereof) comprising 2 distinct (i.e. non-identical) proteinaceaous immunogens disclosed herein wherein the first of said immunogens is SEQ ID NO: 11 or a variant or fragment thereof disclosed herein in combination with a proteinaceous immunogen selected from any one of SEQ ID NOs: 1, 2, 3, 4, 5, 6, 7, 8, 9, 10, 11, 12, 13, 14, 15, and 16 or in combination with a variant or fragment disclosed herein of any one of SEQ ID NOs: 1, 2, 3, 4, 5, 6, 7, 8, 9, 10, 11, 12, 13, 14, 15, and 16.

Also, embodiments of the invention relate to a composition (or the use as a vaccine thereof) comprising 2 distinct (i.e. non-identical) proteinaceaous immunogens disclosed herein wherein the first of said immunogens is SEQ ID NO: 12 or a variant or fragment thereof disclosed herein in combination with a proteinaceous immunogen selected from any one of SEQ ID NOs: 1, 2, 3, 4, 5, 6, 7, 8, 9, 10, 11, 12, 13, 14, 15, and 16 or in combination with a variant or fragment disclosed herein of any one of SEQ ID NOs: 1, 2, 3, 4, 5, 6, 7, 8, 9, 10, 11, 12, 13, 14, 15, and 16.

Also, embodiments of the invention relate to a composition (or the use as a vaccine thereof) comprising 2 distinct (i.e. non-identical) proteinaceaous immunogens disclosed herein wherein the first of said immunogens is SEQ ID NO: 13 or a variant or fragment thereof disclosed herein in combination with a proteinaceous immunogen selected from any one of SEQ ID NOs: 1, 2, 3, 4, 5, 6, 7, 8, 9, 10, 11, 12, 13, 14, 15, and 16 or in combination with a variant or fragment disclosed herein of any one of SEQ ID NOs: 1, 2, 3, 4, 5, 6, 7, 8, 9, 10, 11, 12, 13, 14, 15, and 16.

Also, embodiments of the invention relate to a composition (or the use as a vaccine thereof) comprising 2 distinct (i.e. non-identical) proteinaceaous immunogens disclosed herein wherein the first of said immunogens is SEQ ID NO: 14 or a variant or fragment thereof disclosed herein in combination with a proteinaceous immunogen selected from any one of SEQ ID NOs: 1, 2, 3, 4, 5, 6, 7, 8, 9, 10, 11, 12, 13, 14, 15, and 16 or in combination with a variant or fragment disclosed herein of any one of SEQ ID NOs: 1, 2, 3, 4, 5, 6, 7, 8, 9, 10, 11, 12, 13, 14, 15, and 16.

Also, embodiments of the invention relate to a composition (or the use as a vaccine thereof) comprising 2 distinct (i.e. non-identical) proteinaceaous immunogens disclosed herein wherein the first of said immunogens is SEQ ID NO: 15 or a variant or fragment thereof disclosed herein in combination with a proteinaceous immunogen selected from any one of SEQ ID NOs: 1, 2, 3, 4, 5, 6, 7, 8, 9, 10, 11, 12, 13, 14, 15, and 16 or in combination with a variant or fragment disclosed herein of any one of SEQ ID NOs: 1, 2, 3, 4, 5, 6, 7, 8, 9, 10, 11, 12, 13, 14, 15, and 16.

Also, embodiments of the invention relate to a composition (or the use as a vaccine thereof) comprising 2 distinct (i.e. non-identical) proteinaceaous immunogens disclosed herein wherein the first of said immunogens is SEQ ID NO: 16 or a variant or fragment thereof disclosed herein in combination with a proteinaceous immunogen selected from any one of SEQ ID NOs: 1, 2, 3, 4, 5, 6, 7, 8, 9, 10, 11, 12, 13, 14, 15, and 16 or in combination with a variant or fragment disclosed herein of any one of SEQ ID NOs: 1, 2, 3, 4, 5, 6, 7, 8, 9, 10, 11, 12, 13, 14, 15, and 16.

### Treatment Methods of the Invention

The method of the sixth aspect of the invention generally relates to induction of immunity and as such also entails method that relate to treatment, prophylaxis and amelioration of disease.

When immunization methods entail that a polypeptide of the invention or a composition comprising such a polypeptide is administered the animal (e.g. the human) typically receives between 0.5 and 5,000 μg of the polypeptide of the invention per administration.

In preferred embodiments of the sixth aspect, the immunization scheme includes that the animal (e.g. the human) receives a priming administration and one or more booster administrations.

Preferred embodiments of the 6^{th} aspect of the invention comprise that the administration is for the purpose of inducing protective immunity against S. aureus. In this embodiment it is particularly preferred that the protective immunity is effective in reducing the risk of attracting infection with S. aureus or is effective in treating or ameliorating infection with S. aureus.

As mentioned herein, the preferred vaccines of the invention induce humoral immunity, so it is preferred that the administration is for the purpose of inducing antibodies specific for S. aureus and wherein said antibodies or B-lymphocytes producing said antibodies are subsequently recovered from the animal.

But, as also mentioned the method of the 6^{th} aspect may also be useful in antibody production, so in other embodiments the administration is for the purpose of inducing antibodies specific for S. aureus and wherein B-lymphocytes producing said antibodies are subsequently recovered from the animal and used for preparation of monoclonal antibodies.

Pharmaceutical compositions can as mentioned above comprise polypeptides, antibodies, or nucleic acids of the invention. The pharmaceutical compositions will comprise a therapeutically effective amount thereof.

The term “therapeutically effective amount” or “prophylactically effective amount” as used herein refers to an amount of a therapeutic agent to treat, ameliorate, or prevent a desired disease or condition, or to exhibit a detectable therapeutic or preventative effect. The effect can be detected by, for example, chemical markers or antigen levels. Therapeutic effects also include reduction in physical symptoms, such as decreased body temperature. The precise effective amount for a subject will depend upon the subject's size and health, the nature and extent of the condition, and the therapeutics or combination of therapeutics selected for administration. Thus, it is not useful to specify an exact effective amount in advance. Reference is however made to the ranges for dosages of immunologically effective amounts of polypeptides, cf. above.

However, the effective amount for a given situation can be determined by routine experimentation and is within the judgement of the clinician.

For purposes of the present invention, an effective dose will be from about 0.01 mg/kg to 50 mg/kg or 0.05 mg/kg to about 10 mg/kg of the DNA constructs in the individual to which it is administered.

A pharmaceutical composition can also contain a pharmaceutically acceptable carrier. The term “pharmaceutically acceptable carrier” refers to a carrier for administration of a therapeutic agent, such as antibodies or a polypeptide, genes, and other therapeutic agents. The term refers to any pharmaceutical carrier that does not itself induce the production of antibodies harmful to the individual receiving the composition, and which may be administered without undue toxicity. Suitable carriers may be large, slowly metabolized macromolecules such as proteins, polysaccharides, polylactic acids, polyglycolic acids, polymeric amino acids, amino acid copolymers, and inactive virus particles. Such carriers are well known to those of ordinary skill in the art.

Pharmaceutically acceptable salts can be used therein, for example, mineral acid salts such as hydrochlorides, hydrobromides, phosphates, sulfates, and the like; and the salts of organic acids such as acetates, propionates, malonates, benzoates, and the like. A thorough discussion of pharmaceutically acceptable excipients is available in Remington's Pharmaceutical Sciences (Mack Pub. Co., N.J. 1991).

Pharmaceutically acceptable carriers in therapeutic compositions may contain liquids such as water, saline, glycerol and ethanol. Additionally, auxiliary substances, such as wetting or emulsifying agents, pH buffering substances, and the like, may be present in such vehicles. Typically, the therapeutic compositions are prepared as injectables, either as liquid solutions or suspensions; solid forms suitable for solution in, or suspension in, liquid vehicles prior to injection may also be prepared. Liposomes are included within the definition of a pharmaceutically acceptable carrier.

As is apparent from the claims, the invention also relates to related embodiments to the treatment and prophylaxis disclosed herein: the invention also includes embodiments where
the polypeptide of the invention is for use as a pharmaceutical, in particular for use as a pharmaceutical in the treatment, prophylaxis or amelioration of infection with S. aureus;
the nucleic acid fragment of the invention or the vector of the invention is for use as a pharmaceutical, in particular for use as a pharmaceutical in the treatment, prophylaxis or amelioration of infection with S. aureus;
the transformed cell of the invention is for use as a pharmaceutical, in particular for use as a pharmaceutical in the treatment, prophylaxis or amelioration of infection with S. aureus.
the antibody, antibody fragment or antibody analogue of the invention is for use as a pharmaceutical, in particular for use as a pharmaceutical in the treatment, prophylaxis or amelioration of infection with S. aureus.

### EXAMPLE

### Testing S. aureus Derived Vaccines in Mice Challenged with MRSA

### Expression and Purification of S. aureus Genes

1. Gene fragments that encode the selected S. aureus polypeptides of the invention are prepared synthetically and are introduced into the pQE-1 vector (Qiagen) from Genscript. The fragments are inserted by blunt ended ligation into the the PVU II site in the 5′-end, immediately following the vector's coding region for the 6 histidinyl residues. In the 3′-end, all inserted gene fragments include a stop codon.
2. The vectors from 1 are transfected into the E. Coli M15[pREP4] strain, which contains an expression as well as a repressor plasmid facilitating proper expression.
3. The vectors from 1 are further inserted into the E. coli XL1 Blue for long-time storage.
4. The transfected and selected clones are tested for expression in small scale whereby optimum conditions for expression in terms of the amount of IPTG, the density of cells and the time of expression induction are determined.
5. From the information obtained in 4, large scale cultures are established; subsequently the expression products are harvested and purified on a Ni-NTA column.
6. Purity and yield of the large-scale expression is investigated by means of SDS-PAGE and spectrophotometry, whereafter the proteins are aliquoted for use in immunization experiments and other experiments.

### Immunization and S. aureus Challenge Infection in Mice

1. 2 months old NMRI mice were used.
2. Groups of at least 8 mice were used for immunization. The mice were immunized 3 times (at day 0, 14, and 28) prior to challenge infection. A control group was treated according to an identical protocol with the exception that an irrelevant protein antigen or phosphate buffered saline was used for immunization.

### 1st Immunization:

25 μg protein (per mice) was mixed with 100 μl aluminum hydroxide (Alhydrogel 2.0%, Brenntag) per 125 ug protein and incubated with end-over-end rotation for 15 min. Freund's incomplete adjuvant (sigma) was added in the volume 1:1 and the mixture was vortexed thoroughly for 1 hour. This mixture was injected subcutaneously.

### 2nd and 3rd Immunization:

The mice were booser injected intraperitoneally with 2 weeks interval, using the same amount of protein mixed with aluminum hydroxide and physiological saline solution.
3. 14 days after the last immunization, a number of bacteria (2×10⁹ cells) corresponding to a predetermined LD 90 in the control group of mice was administered intraperitoneally to all mice.

The cells were handled cold and kept on ice until use. The stock solution of MRSA cells were thawed on ice and then the appropriate amount of cells was diluted in sterile physiological saline (total volume per mouse 500 μl).

The survival was surveilled twice daily in the first 48 hours after challenge and once daily in the subsequent 7 days. The mice were sacrificed if they showed signs of suffering. The mice were monitored with respect to loss of weight and body temperature using an implanted chip. The organs of the mice were used for determination of CFU counts for for SAR1402 (SEQ ID NO: 5), SAR 2723 (SEQ ID NO: 13) and SAR2753 (SEQ ID NO: 14).

The results from 7 polypeptide vaccinations are presented in the Figures.

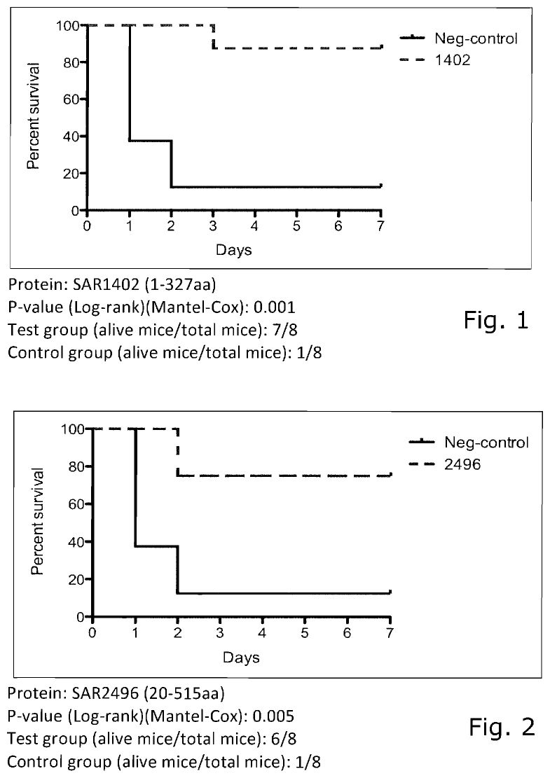

FIG. 1 shows the survival curves for 8 mice immunized with full-length SAR1402 (SEQ ID NO: 5) and 8 mice immunized with negative control. Survival in the vaccinated group at day 7 was ⅞ mice, whereas only ⅛ mice in the control group survived. The increased survival in the vaccinated group is statistically highly significant (P=0.001 according to a Log-rank Mantel-Cox test).

FIG. 2 shows the survival curves for 8 mice immunized with amino acids 20-515 of SAR2496 (SEQ ID NO: 11) and 8 mice immunized with negative control. Survival in the vaccinated group at day 7 was 6/8 mice, whereas only ⅛ mice in the control group survived. The increased survival in the vaccinated group is statistically highly significant (P=0.005 according to a Log-rank Mantel-Cox test).

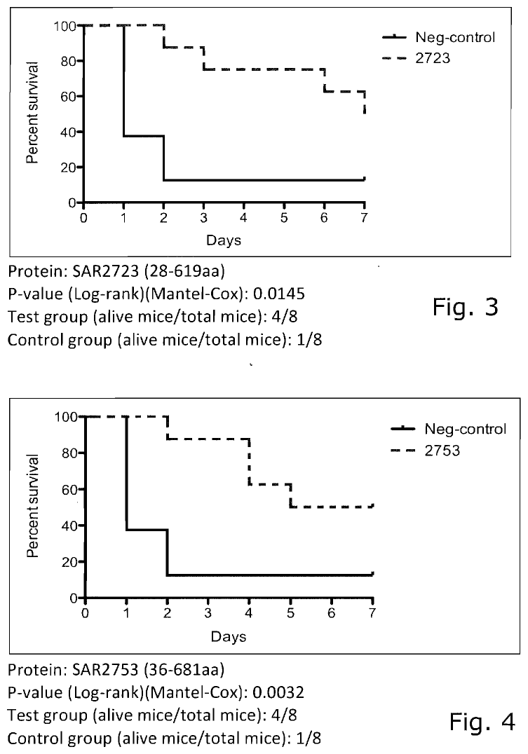

FIG. 3 shows the survival curves for 8 mice immunized with amino acids 28-619 of SAR2723 (SEQ ID NO: 13) and 8 mice immunized with negative control. Survival in the vaccinated group at day 7 was 4/8 mice, whereas only ⅛ mice in the control group survived. The increased survival in the vaccinated group is statistically highly significant (P=0.0145 according to a Log-rank Mantel-Cox test).

FIG. 4 shows the survival curves for 8 mice immunized with amino acids 36-681 of SAR2753 (SEQ ID NO: 14) and 8 mice immunized with negative control. Survival in the vaccinated group at day 7 was 4/8 mice, whereas only ⅛ mice in the control group survived. The increased survival in the vaccinated group is statistically highly significant (P=0.0032 according to a Log-rank Mantel-Cox test).

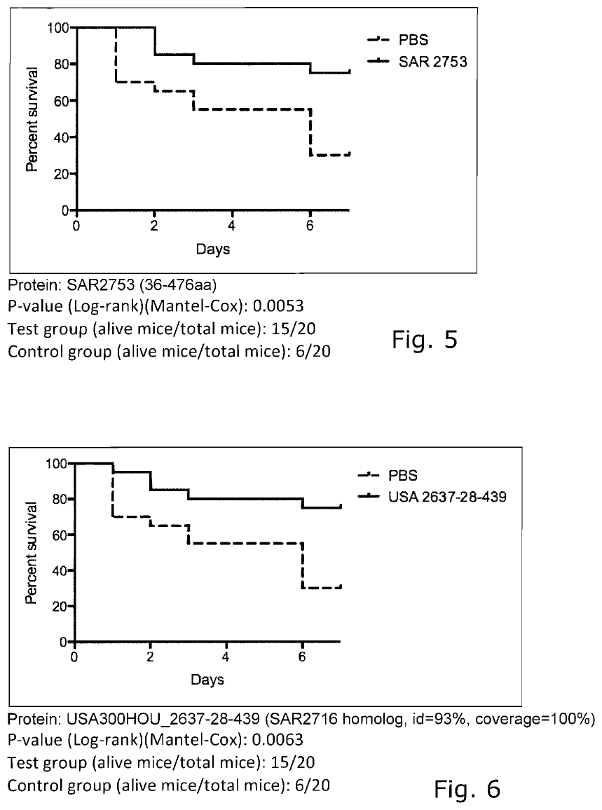

FIG. 5 shows the survival curves for 20 mice immunized with amino acids 28-619 of SAR2723 (SEQ ID NO: 13) and 20 mice immunized with negative control (phosphate buffered saline, PBS). Survival in the vaccinated group at day 7 was 15/20 mice, whereas only 6/20 mice in the control group survived. The increased survival in the vaccinated group is statistically highly significant (P=0.0053 according to a Log-rank Mantel-Cox test).

FIG. 6 shows the survival curves for 20 mice immunized with a homologue of SAR2716 (USA300HOU_2637_28_439 shown in SEQ ID NO: 49) and 20 mice immunized with negative control (PBS). Survival in the vaccinated group at day 7 was 15/20 mice, whereas only 6/20 mice in the control group survived. The increased survival in the vaccinated group is statistically highly significant (P=0.0063 according to a Log-rank Mantel-Cox test). USA300HOU_2637_28_439 has a sequence identity with SEQ ID NO: 10, residues 28-439, of 93%.

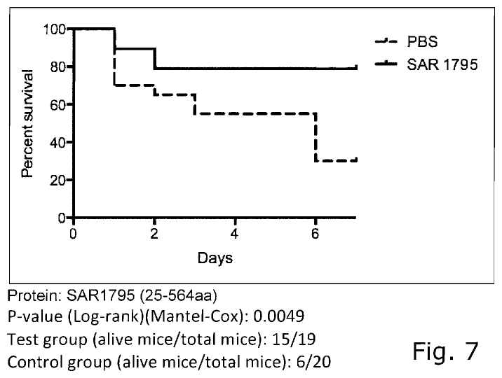

FIG. 7 shows the survival curves for 19 mice immunized with amino acids 25-564 of SAR1795 (SEQ ID NO: 12) and 20 mice immunized with negative control (PBS). Survival in the vaccinated group at day 7 was 15/19 mice, whereas only 6/20 mice in the control group survived. The increased survival in the vaccinated group is statistically highly significant (P=0.0049 according to a Log-rank Mantel-Cox test).

### SEQUENCE INFORMATION

The sequence listing included sets forth the sequences of polypeptides and nucleic acids of the present invention. For easy reference, the sequences are presented in the following:

The polypeptides of the present invention have the following amino acid sequences:

SEQ IN NO: 1
MAKGNLFKAILGIGGAVAAVLVTRKDSRDKLKAEYNKYKQDPQSYKDNAKDKATQLGTIANIETIKEVK
TNPKEYANRLKNNPKAFFEEEKSKFTEYDNKTDESIEKGKFDDEGGAAPNNNLRIVTEEDLKKNKNALS
DKE
SEQ IN NO: 2
MKKLVSIVGATLLLAGCGSQNLAPLEEKTTDLREDNHQLKLDIQELNQQISDSKSKIKGLEKDKENSKK
TASNNTKIKLMNVTSTYYDKVAKALKSYNDIEKDVSKIVKGDKNVQSKLNQISNDIQSAHTSYKDAIDG
LSLSDDDKKTSKNIDKLNSDLNHAFDDIKNGYQNKDKKQLTKGQQALSKLNLNAKS
SEQ IN NO: 3
MKKLVTGLLALSLFLAACGQDSDQQKDSNKEKDDKAKTEQQDKKTNDSSKDKKDNKDDSKDVNKDNKDN
SANDNQQQSNSNATNNDQNQTNNNQSNSGQTTNNQKSSYVAPYYGQNAAPVARQIYPFNGNKSQALQQL
PNFQTALNAANNEANKFGNGHKVYNIDYSIEEHNGNYKYVFSFKDPNVNGKYSIVTVDYTGQAMVTDPN
YQQ
SEQ IN NO: 4
MKKVMGILLASTLILGACGHHQDSAKKESTSHKKKENIDNEELNEELKEFKSKKNMDIKIKGDTIVSDK
FEAKIKEPFIINEKDEKKKYIAFKMEITAKKDDKDLNPSSISHDYINITQDDKNTVNKLRDGYLLSDKN
YKDWTEHNQDQIKKGKTAQAMFIYELRGDGNINLNVHKYSEDKTVDSKSFKFSKLKTEDFSHRAETREE
VEKKEKEFEEEYKKEQEREKEKEKQKDDDHSGLDEV
SEQ IN NO: 5
MKKWQFVGTTALGATLLLGACGGGNGGSGNSDLKGEAKGDGSSTVAPIVEKLNEKWAQDHSDAKISAGQ
AGTGAGFQKFIAGDIDFADASRPIKDEEKQKLQDKNIKYKEFKIAQDGVTVAVNKENDFVDELDKQQLK
AIYSGKAKTWKDVNSKWPDKKINAVSPNSSHGTYDFFENEVMNKEDIKAEKNADTNAIVSSVTKNKEGI
GYFGYNFYVQNKDKLKEVKIKDENGKATEPTKKTIQDNSYALSRPLFIYVNEKALKDNKVMSEFIKFVL
EDKGKAAEEGGYVAAPEKTYKSQLDDLKAFIDKNQKSDDKKSDDKKSEDKK
SEQ IN NO: 6
MKGKFLKVSSLFVATLTTATLVSSPAANALSSKAMDNHPQQTQTDKQQTPKIQKGGNLKPLEQRERANV
ILPNNDRHQITDTTNGHYAPVTYIQVEAPTGTFIASGVVVGKDTLLTNKHIVDATHGDPHALKAFASAI
NQDNYPNGGFTAEQITKYSGEGDLAIVKFSPNEQNKHIGEVVKPATMSNNAETQVNQNITVTGYPGDKP
VATMWESKGKITYLKGEAMQYDLSTTGGNSGSPVFNEKNEVIGIHWGGVPNQFNGAVFINENVRNFLKQ
NIEDINFANDDHPNNPDNPDNPNNPDNPNNPDNPNNPDNPDNPNNPDNPNNPDNIPNNPDQPNINPNIN
PDNGDNNNSDNPDAA
SEQ IN NO: 7
MKRTLVLLITAIFILAACGNHKDDQAGKDNQKHNNSSNQVKEIATDKNVQGDNYRTLLPFKESQARGLL
QDNMANSYNGGDFEDGLLNLSKEVFPTDKYLYQDGQFLDKKTINAYLNLKYTKREIDKMSEKDKKDKKA
NENLGLNPSHEGETDPEKIAEKSPAYLSNILEQDFYGGGDTKGKNIKGMTIGLAMNSVYYYKKEKDGPT
FSKKLDDSEVKKQGKQMASEILSRLRENDDLKDIPIHFAIYKQSSEDSITPGEFITQATAEKSQTKLNE
WHNINEKSALLPSSTAADYDENLNNNFKQFNDNLQSYFSNIFTQAVGKVKFVDKKPQRLVVDLPIDYYG
QAETIGITQYVTEQANKYFDKIDNYEIRIKDGNQPRALISKTKDDKEPQVHIYSN
SEQ IN NO: 8
MRENFKLRKMKVGLVSVAITMLYIMTNGQAEASEANEKPSTNQESKVVSQTEQNSKETKTVESNKNFVK
LDTIKPGAQKITGTTLPNHYVLLTVDGKSADSVENGGLGFVEANDKGEFEYPLNNRKIVHNQEIEVSSS
SPDLGEDEEDEEVEEASTDKAGVEEESTEAKVTYTTPRYEKAYEIPKEQLKEKDGHHQVFIEPITEGSG
IIKGHTSVKGKVALSINNKENFEESVKGGVSKEDTKASSDGIWMPIDDKGYFNFDFKTKRFDNLELKEG
NDISLTFAPDDEEDALKPLIFKTKVTSLEDIDKAETKYDHTKLNKVKVLDNVKEDLHVDEIYGSLYHTD
KGKGILDKEGTKVIKGKTKFANAVVKVDSELGEAQLFPDLQVNEKGEFSFDSHGAGFRLQNGEKLNFTV
VDPITGDLLSNEFVSKEIDIEETPEQKADREFDEKLENTPAYYKLYGDKIVGFDTNDFPITWFYPLGEK
KVERTTPKLEK
SEQ IN NO: 9
MSKKLKIIIPIIIVLLLIGGIAWGVYAFFANTPKNTYLKSEQQTAKMYKDYFNDRFENEVKFQEKMKDN
SFLSSLELSADASDEIVKGLGIPKSVVNASKIKMSYGHDPKKEKSMINLEPTIADSALGKFQLAADKDK
HYFESPLFKGKYSVNNSDLLSTYSKLTGEDEETAKENGITNQQLNLNTLFSNAQAQQSDYSKIAEKYSE
LIVDKLDDDNFDKGKKEEIKVNGEKYKVRPVTLTLSRADTKKITLAVLEEAKKDKDLKKLMEEQGTTKD
FEKDIKKAIDDVKETKKDEFAKIQSKIYTEKHTIVKREITITDKENNKTKIKGTNTLEDDKLKLDYALD
FDQDKYTYAEAKYTIKGVSSKEKDNKYSDKYEFGKKTEYDESKIKLDNQEKVDGTKRQDKGKITVALDK
YSDENEFTFENNIDSDVKNNTQKSTLNIGIKYAEEPINFILKSSTKLKADIDFDDSGAKDFNSLSSKDR
EKLEKEIEKNGGKMFESILKKASK
SEQ IN NO: 10
MRKFSRYAFTSMATVTLLSSLTPAALASDTNHKPATSDINFEITQKSDAVKALKELPKSENVKNHYQDY
SVTDVKTDKKGFTHYTLQPSVDGVHAPDKEVKVHADKSGKVVLINGDTDAKKVKPTNKVTLSKDEAADK
AFNAVKIDKNKAKNLQDDVIKENKVEIDGDSNKYIYNIELITVTPEISHWKVKIDADTGAVVEKTNLVK
EAAATGTGKGVLGDTKDININSIDGGFSLEDLTHQGKLSAYNFNDQTGQATLITNEDENFVKDDQRAGV
DANYYAKQTYDYYKNTFGRESYDNHGSPIVSLTHVNHYGGQDNRNNAAWIGDKMIYGDGDGRTFTNLSG
ANDVVAHELTHGVTQETANLEYKDQSGALNESFSDVFGYFVDDEDFLMGEDVYTPGKEGDALRSMSNPE
QFGQPSHMKDYVYTEKDNGGVHTNSGIPNKAAYNVIQAIGKSKSEQIYYRALTEYLTSNSNFKDCKDAL
YQAAKDLYDEQTAEQVYEAWNEVGVE
SEQ IN NO: 11
MKKKLGMLLLVPAVTLSLAACGNDDGKDKDGKVTIKTTVYPLQSFAEQIGGKHVKVSSIYPAGTDLHSY
EPTQKDILSASKSDLFMYTGDNLDPVAKKVASTIKDKDKKLSLEDKLDKAKLLTDQHEHGEEHEHEGHD
HGKEEHHHHGGYDPHVWLDPKINQTFAKEIKDELVKKDPKHKDDYEKNYKKLNDDLKKIDNDMKQVTKD
KQGNAVFISHESIGYLADRYGFVQKGIQNMNAEDPSQKELTKIVKEIRDSNAKYILYEDNVANKVTETI
RKETDAKPLKFYNMESLNKEQQKKDNITYQSLMKSNIENIGKALDSGVKVKDDKAESKHDKAISDGYFK
DEQVKDRELSDYAGEWQSVYPYLKDGILDEVMEHKAENDPKKSAKDLKAYYDKGYKTDITNIDIKGNEI
TFTKDGTKHTGKYEYNGKKTLKYPKGNRGVRFMFKLVDGNDKDLPKFIQFSDHNIAPKKAEHFHIFMGN
DNDALLKEMDNWPTYYPSKLNKDQIKEEMLAH
SEQ IN NO: 12
MVLYIILAIIVIILIAVGVLFYLRSNKRQIIEKAIERKNEIETLPFDQNLAQLSKLNLKGETKTKYDAM
KKDNVESINKYLAPVEEKIHNAEALLDKFSFNASQCEIDDANELMDSYEQSYQQQLEDVNEIIALYKDN
DELYDKCKVDYREMKRDVLANRHQFGEAASLLETEIEKFEPRLEQYEVLKADGNYVQAHNHIAALNEQM
KQLRSYMEEIPELIRETQKELPGQFQDLKYGCRDLKVEGYDLDHVKVDSTLQSLKTELSFVEPLISRLE
LEEANDKLANINDKLDDMYDLIEHEVKAKNDVEETKDIITDNLFKAKDMNYTLQTEIEYVRENYYINES
DAQSVRQFENEIQSLISVYDDILKEMSKSAVRYSEVQDNLQYLEDHVTVINDKQEKLQNHLIQLREDEA
EAEDNILLRVQSKKEEVYRRLLASNLTSVPERFIIMKNEIDHEVRDVNEQFSERPIHVKQLKDKVSKIV
IQMNTFEDEANDVLVNAVYAEKLIQYGNRYRKDYSNVDKSLNEAERLFKNINRYKRAIEIAEQALESVE
PGVTKHIEEEVIKQ
SEQ IN NO: 13
MPKNKILIYLLSTTLVLPTLVSPTAYADTPQKDTTAKTTSHDSKKSTDDETSKDTTSKDIDKADNNNTS
NQDNNDKKVKTIDDSTSDSNNIIDFIYKNLPQTNINQLLTKNKYDDNYSLTTLIQNLFNLNSDISDYEQ
PRNGEKSTNDSNKNSDNSIKNDTDTQSSKQDKADNQKAPKSNNTKPSTSNKQPNSPKPTQPNQSNSQPA
SDDKVNQKSSSKDNQSMSDSALDSILDQYSEDAKKTQKDYASQSKKDKNEKSNTKNPQLPTQDELKHKS
KPAQSFNNDVNQKDIRATSLFETDPSISNNDDSGQFNVVDSKDTRQFVKSIAKDAHRIGQDNDIYASVM
IAQAILESDSGRSALAKSPNHNLFGIKGAFEGNSVPFNTLEADGNQLYSINAGFRKYPSTKESLKDYSD
LIKNGIDGNRTIYKPTWKSEADSYKDATSHLSKTYATDPNYAKKLNSIIKHYQLTQFDDERMPDLDKYE
RSIKDYDDSSDEFKPFREVSDNMPYPHGQCTWYVYNRMKQFGTSISGDLGDAHNWNNRAQYRDYQVSHT
PKRHAAVVFEAGQFGADQHYGHVAFVEKVNSDGSIVISESNVKGLGIISHRTINAAAAEELSYITGK
SEQ IN NO: 14
MMKSQNKYSIRKFSVGASSILIATLLFLSGGQAQAAEKQVNMGNSQEDTVTAQSIGDQQTRENANYQRE
NGVDEQQHTENLTKNLHNDKTISEENHRKTDDLNKDQLKDDKKSSLNNKNIQRDTTKNNNANPRDVNQG
LEQAINDGKQSKVASQQQSKEADNSQDLNANNNLPSQSRTKVSPSLNKSDQTSQREIVNETEIEKVQPQ
QKNQANDKITDHNFNNEQEVKPQKDEKTLSVSDLKNNQKSPVEPTKDNDKKNGLNLLKSSAVATLPNKG
TKELTAKAKGDQTNKVAKQGQYKNQDPIVLVHGFNGFTDDINPSVLAHYWGGNKMNIRQDLEENGYKAY
EASISAFGSNYDRAVELYYYIKGGRVDYGAAHAAKYGHERYGKTYEGIYKDWKPGQKVHLVGHSMGGQT
IRQLEELLRNGSREEIEYQKKHSGEISPLFKGNNDNMISSITTLGTPHNGTHASDLAGNEALVRQIVFD
IGKMFGNKNSRVDFGLAQWGLKQKPNESYIDYVKRVKQSNLWKSKDNGFYDLTREGATDLNRKTSLNPN
IVYKTYTGEATHKALNSDRQKADLNMFFPFVITGNLIGKATEKEWRENDGLVSVISSQHPFNQAYTNAT
DKIQKGIWQVTPTKHDWDHVDFVGQDSSDTVRTREELQDFWHHLADDLVKTEKVTDTKQA
SEQ IN NO: 15
MTNKMKKWQKLSTITLLMTGVIALNNGEFRNVDKHQIAVADTNVQTPDYEKLKKTWLDVNYGYDQYDEN
NQDMKKKFDAKEKEAKKLLDDMKTDTNRTYLWSGAENLETNSSHMTKTYRNIEKIAESMQHKNTVLKTV
ENKLKIKEALDWMNKNVYGKNPSQKVEDLTKNRKGQTTPKNNSLNWWDYEIGTPRALTNTLLLMDDMLT
KDEMKNYSKPISTYAPSSDKILSSVGESEDAKGGNLVDISKVKLLESVIEEDVDNILKKSIDSFNKVFT
YVQDSATGKGRNGFYKDGSYIDHQDVPYTGAYGVVLLEGISQMMPMIKESPFKTTQDNATLSNWIDEGF
MPLIYKGEMMDLSRGRAISRENETSHTASATVMKSLLRLNDTMDDSTKTRYKQIVKTSVNSDSSYNQNN
YLNSYSDIAKMKKLMNDSTISKNDLTQQLKIYNDMDRVTYHNKDLDFAFGLSMTSKNIARYENINGENL
KGWHTGAGMSYLYNSDVKHYRDNFWATADMTCLPGTTTLNDMPSTNTKNDKSFVGGTKLNNKYASIGMD
FENQDKTLTAKKSYFILNIDKIVFLGTGIKSTDSSKNPVTSVENRKANGYKLFKDDIEITTSDVNAQET
HSVFLESNIDTKKNIGYHFLDKPKITVKKESHTGKWSEINKSQKKDDKKDEYYEVTQTHNTSDSKYAYV
LYPGLSKSDFKSKNNNVSIVKQDEDFHVIKDNDGVFAGVNYSDNTKSFDINGITVELKEKGMFVIKKKD
DKAYKCSFYNPETTNTASNIESKIFIKGYTITNKSVINSNDAGVNFELTK
SEQ IN NO: 16
MTYRMKKWQKLSTITLLMAGVITLNGGEFRSIDKHQIAVADTNVQTTDYEKLRNIWLDVNYGYDKYDEN
NPDMKKKFEATENEAEKLLKEMKTESDRKYLWESSKDLDTKSADMTRTYRNIEKISEAMKHKNTKLKTD
ENKTKVKDALEWLHKNAYGKEPDKKVADLTSNFKNKTSRNTNLNWWDYEIGTPRALTNTLILLQEDFTD
EEKKKYTAPIKTFAPDSDKILSSVGKSEPAKGGNLVDISKVKLLESIIEEDKDMMKKSIDSFNITVFTY
AQNSATGKERNGFYKDGSYIDHQDVPYTGAYGVVLLEGISQMMPMIKETPFNDSNQNDTTLKSWIDDGF
MPLIYKGEMMDLSRGRAISRENETSHSASATVMKSLLRLSDTMDKSTKAKYKKIVKTSVESDSSYKQTD
YLSSYSDISKMKSLMEDSTISTNGLTQQLKIYNDMDRVTYHNKGLDFAFGLSMTSKNVARYESINGENL
KGWHTGAGMSYLYNSDVKHYRDNFWATADMKRLAGTTTLDNEEPKSTDVKKSSKTFVGGTKFDDQHASI
GMDFENQDKTLTAKKSYFILNDKIVFLGTGIKSTDSSKNPVTTIENRKANDYKLYKDDTQTTNSDNQET
NSLFLESTNSTQNNIGYHFLNESKITVKKESHTGKWSDINKSQKDIQKTDEYYEVTQKHSNTDSKYAYV
LYPGLSKDVFKSKASKVTVVKQEDDFHVVKDNESVWAGINYSDSAKTFEINNTKVEVKAKGMFILTKKD
DNTYECSFYNPESTNSVSDIESKISMTGYSIINKNTSTSNESGVRFELTK
SEQ ID NO: 49:
IDSKNKPANSDIKFEVTQKSDAVKALKELPKSENVKNIYQDYAVTDVKTDKKGFTHYTLQPSVDGVHAP
DKEVKVHADKSGKVVLINGDTDAKKVKPTNKVTLSKDDAADKAFKAVKIDKIVKAKNLKDKVIKENKVE
IDGDSNKYVYNVELITVTPEISHWKVKIDAQTGEILEKMNLVKEAAETGKGKGVLGDTKDININSIDGG
FSLEDLTHQGKLSAFSFNDQTGQATLITNEDENFVKDEQRAGVDANYYAKQTYDYYKDTFGRESYDNQG
SPIVSLTHVNNYGGQDNIRNNAAWIGDKMIYGDGDGRTFTSLSGANDVVAHELTHGVTQETANLEYKDQ
SGALNESFSDVFGYFVDDEDFLMGEDVYTPGKEGDALRSMSNPEQFGQPAHNIKDYVFTEKDNGGVHTN
S

The nucleic acid fragments of the present invention have the following sequences:

SEQ IN NO: 17
ATGGCAAAAGGTAATTTATTTAAAGCGATTTTAGGTATAGGTGGCGCTGTAGCAGCTGTACTTGTTACACG
TAAAGATAGTCGTGACAAGCTGAAAGCAGAATATAATAAATACAAACAAGATCCTCAAAGCTATAAAGATA
ATGCTAAGGATAAAGCGACGCAATTAGGAACAATTGCAAATGAAACAATTAAAGAAGTAAAAACAAATCCG
AAAGAATATGCTAATAGATTAAAAAATAATCCAAAAGCATTTTTCGAAGAAGAAAAATCAAAATTTACCGA
ATATGACAATAAGACTGACGAAAGTATTGAAAAAGGTAAATTTGATGATGAAGGTGGCGCAGCACCAAATA
ATAATTTACGTATCGTCACTGAAGAAGATTTAAAAAAGAATAAAAATGCATTGTCTGATAAAGAATAA
SEQ IN NO: 18
ATGAAAAAATTGGTTTCAATTGTTGGCGCAACATTATTGTTAGCTGGATGTGGATCACAAAATTTAGCACC
ATTAGAAGAAAAAACAACAGATTTAAGAGAAGATAATCATCAACTCAAACTAGATATTCAAGAACTTAATC
AACAAATTAGTGATTCTAAATCTAAAATTAAAGGGCTTGAAAAGGATAAAGAAAATAGTAAAAAAACTGCA
TCTAATAATACGAAAATTAAATTGATGAATGTTACATCAACATACTACGACAAAGTTGCTAAAGCTTTGAA
ATCCTATAACGATATTGAAAAGGATGTAAGTAAAAACAAAGGCGATAAGAATGTTCAATCGAAATTAAATC
AAATTTCTAATGATATTCAAAGTGCTCACACTTCATACAAAGATGCTATCGATGGTTTATCACTTAGTGAT
GATGATAAAAAAACGTCTAAAAATATCGATAAATTAAACTCTGATTTGAATCATGCATTTGATGATATTAA
AAATGGCTATCAAAATAAAGATAAAAAACAACTTACAAAAGGACAACAAGCGTTGTCAAAATTAAACTTAA
ATGCAAAATCATGA
SEQ IN NO: 19
ATGAAAAAATTAGTTACAGGGTTATTAGCATTATCATTATTTTTAGCTGCATGTGGTCAAGATAGTGACCA
ACAAAAAGACAGTAATAAAGAAAAAGATGATAAAGCGAAAACTGAACAACAAGATAAAAAAACAAATGATT
CATCTAAAGATAAGAAAGACAATAAAGATGATAGTAAAGACGTAAACAAAGATAATAAAGATAATAGTGCA
AACGATAACCAGCAACAATCTAATTCAAATGCAACAAACAATGACCAAAATCAAACGAATAATAACCAGTC
AAACAGTGGACAAACGACTAACAATCAAAAATCAAGTTACGTTGCACCATATTATGGACAAAACGCAGCGC
CAGTGGCTCGTCAAATTTATCCATTTAATGGTAATAAATCACAAGCATTACAACAATTGCCTAATTTCCAA
ACAGCTTTAAATGCAGCTAACAACGAAGCAAATAAATTTGGTAATGGTCATAAAGTTTATAATGATTATTC
AATTGAAGAACATAATGGTAACTATAAGTATGTTTTTAGTTTTAAAGACCCAAACGTAAATGGAAAATATT
CAATTGTAACGGTTGATTATACTGGACAAGCAATGGTTACTGATCCAAACTACCAACAATAA
SEQ IN NO: 20
ATGAAAAAAGTAATGGGGATATTATTAGCAAGTACACTTATCTTAGGTGCTTGTGGACATCATCAAGATAG
TGCAAAAAAAGAGAGCACTAGTCACAAAAAGAAAGAAAATGACAATGAAGAATTAAATGAAGAACTTAAAG
AATTTAAAAGCAAAAAAAATATGGATATAAAAATTAAAGGCGATACTATTGTTAGTGACAAATTTGAAGCT
AAAATAAAAGAACCGTTTATCATCAATGAAAAAGATGAGAAAAAGAAATATATCGCTTTTAAAATGGAAAT
TACTGCTAAAAAAGACGATAAAGATTTAAATCCATCTTCTATTTCTCATGACTATATTAATATCACTCAAG
ATGATAAAAATACAGTAAATAAATTAAGAGATGGTTATCTTTTAAGTGATAAAAATTATAAAGATTGGACA
GAACATAACCAAGATCAAATTAAAAAAGGCAAAACTGCACAAGCCATGTTTATCTATGAGTTAAGAGGTGA
TGGAAACATTAATTTAAATGTCCATAAATACTCAGAAGATAAAACAGTTGATTCTAAATCATTCAAATTTA
GTAAACTTAAAACCGAAGATTTTTCTCATAGAGCGGAAACAAGGGAAGAAGTAGAAAAGAAAGAAAAAGAA
TTTGAAGAAGAGTACAAAAAAGAACAAGAACGAGAGAAAGAAAAAGAAAAGCAAAAAGATGACGACCACAG
TGGTTTAGATGAAGTATAA
SEQ IN NO: 21
ATGAAAAAATGGCAATTTGTTGGTACTACAGCTTTAGGTGCAACACTATTATTAGGTGCTTGTGGTGGCGG
TAATGGTGGCAGTGGTAATAGTGATTTAAAAGGGGAAGCTAAAGGGGATGGCTCATCAACAGTAGCACCAA
TTGTGGAGAAATTAAATGAAAAATGGGCTCAAGATCACTCGGATGCTAAAATCTCAGCAGGACAAGCTGGT
ACAGGTGCTGGTTTCCAAAAATTCATTGCAGGAGATATCGACTTCGCTGATGCTTCTAGACCAATTAAAGA
TGAAGAGAAGCAAAAATTACAAGATAAGAATATCAAATACAAAGAATTCAAAATTGCGCAAGATGGTGTAA
CGGTTGCTGTAAATAAAGAAAATGATTTTGTAGATGAATTAGACAAACAGCAATTAAAAGCAATTTATTCT
GGAAAAGCTAAAACATGGAAAGATGTTAATAGTAAATGGCCAGATAAAAAAATAAATGCTGTATCACCAAA
CTCAAGTCATGGTACTTATGACTTCTTTGAAAATGAAGTAATGAATAAAGAAGATATTAAAGCAGAAAAAA
ATGCTGATACAAATGCTATCGTTTCTTCTGTAACGAAAAACAAAGAGGGAATCGGATACTTTGGATATAAC
TTCTACGTACAAAATAAAGATAAATTAAAAGAAGTTAAAATCAAAGATGAAAATGGTAAAGCAACAGAGCC
TACGAAAAAAACAATTCAAGATAACTCTTATGCATTAAGTAGACCATTATTCATTTATGTAAATGAAAAAG
CATTGAAAGATAATAAAGTAATGTCAGAATTTATCAAATTCGTCTTAGAAGATAAAGGTAAAGCAGCTGAA
GAAGGTGGATATGTAGCAGCACCAGAGAAAACATACAAATCACAATTAGATGATTTAAAAGCATTTATTGA
TAAAAATCAAAAATCAGACGACAAGAAATCTGATGATAAAAAGTCTGAAGACAAGAAATAA
SEQ IN NO: 22
ATGAAAGGTAAATTTTTAAAAGTTAGTTCTTTATTCGTTGCAACTTTGACAACAGCGACACTTGTGAGTTC
TCCAGCAGCAAATGCGTTATCTTCAAAAGCTATGGACAATCATCCACAACAAACGCAGACAGACAAACAGC
AAACACCTAAGATTCAAAAAGGCGGTAACCTTAAACCATTAGAACAACGTGAACGCGCTAATGTTATATTA
CCAAATAACGATCGTCACCAAATCACAGATACAACGAATGGTCATTATGCACCTGTTACTTATATTCAAGT
TGAAGCACCTACTGGTACATTTATTGCTTCTGGTGTAGTTGTAGGTAAAGATACACTTTTAACAAATAAAC
ACATCGTAGATGCTACGCACGGTGATCCTCATGCTTTAAAAGCATTCGCTTCTGCAATTAACCAAGACAAT
TATCCTAATGGTGGTTTCACTGCTGAACAAATCACTAAATATTCAGGCGAAGGTGATTTAGCAATCGTTAA
ATTCTCCCCTAATGAGCAAAACAAACATATTGGCGAAGTAGTTAAACCAGCAACAATGAGTAATAATGCTG
AAACACAAGTTAACCAAAATATTACTGTAACAGGATATCCTGGTGATAAACCTGTCGCAACAATGTGGGAA
AGTAAAGGAAAAATAACGTACTTAAAAGGTGAAGCAATGCAATATGATTTAAGTACAACTGGTGGTAACTC
AGGTTCACCTGTATTTAATGAAAAAAATGAAGTCATTGGCATTCATTGGGGTGGCGTTCCAAATCAATTTA
ACGGTGCAGTATTTATTAATGAAAATGTACGCAACTTCTTAAAACAAAATATTGAAGATATCAATTTCGCA
AATGATGACCACCCTAACAACCCTGATAATCCAGACAATCCAAATAATCCGGACAATCCTAACAACCCTGA
TAACCCTAACAACCCTGATAATCCAGACAATCCTAATAATCCTGATAACCCTAACAACCCGGACAATCCAA
ATAACCCTGACCAACCTAACAACCCAAATAACCCGGACAATGGCGATAACAATAATTCAGACAACCCTGAC
GCTGCATAA
SEQ IN NO: 23
ATGAAGCGTACATTAGTATTATTGATTACAGCTATCTTTATACTCGCTGCTTGTGGTAACCATAAGGATGA
CCAGGCTGGAAAAGATAATCAAAAACATAACAATAGTTCAAATCAAGTAAAAGAAATTGCTACGGATAAAA
ATGTACAAGGTGATAACTATCGTACATTGTTACCATTTAAAGAAAGCCAGGCAAGAGGACTTTTACAAGAT
AACATGGCAAATAGTTATAATGGCGGCGACTTTGAAGATGGTTTATTGAACTTAAGTAAAGAAGTGTTTCC
AACAGACAAATATTTGTATCAAGATGGTCAATTTTTGGACAAGAAAACAATTAATGCCTATTTAAATCTTA
AGTATACAAAACGTGAAATCGATAAAATGTCTGAAAAAGATAAAAAAGACAAGAAAGCGAATGAAAATTTA
GGACTTAATCCATCACACGAAGGTGAAACAGATCCTGAAAAGATTGCAGAAAAATCACCAGCCTATTTATC
TAACATTTTAGAGCAAGATTTTTATGGTGGTGGAGATACAAAAGGTAAGAATATTAAAGGTATGACGATTG
GTTTAGCTATGAATAGTGTTTATTACTATAAAAAAGAAAAAGATGGACCGACTTTTAGTAAAAAACTAGAT
GATAGCGAAGTTAAAAAGCAAGGTAAACAAATGGCTAGTGAGATATTATCAAGGTTACGTGAAAATGATGA
TTTAAAAGATATACCAATTCATTTTGCAATTTATAAGCAATCAAGTGAAGATTCAATCACACCAGGTGAAT
TTATCACTCAAGCGACTGCAGAAAAGAGTCAAACAAAGCTTAATGAATGGCATAATATCAATGAAAAATCA
GCTTTATTACCTTCTTCAACAGCAGCAGATTATGATGAAAATTTAAATAATAATTTCAAGCAATTTAATGA
TAATTTGCAATCATATTTTTCTAATTTCACACAAGCAGTAGGAAAAGTTAAATTTGTTGATAAAAAGCCAC
AACGATTAGTAGTAGATTTACCAATCGATTACTATGGACAAGCTGAAACAATTGGTATTACACAGTACGTT
ACTGAACAAGCGAATAAATATTTCGATAAAATCGATAACTATGAAATTCGGATTAAAGATGGTAACCAACC
ACGTGCTTTAATTAGTAAGACAAAAGATGACAAAGAACCGCAAGTTCATATTTACAGTAATTAA
SEQ IN NO: 24
ATGAGGGAAAATTTTAAGTTACGTAAAATGAAAGTCGGGTTAGTATCTGTTGCAATTACAATGTTATATAT
CATGACAAACGGACAAGCAGAAGCATCAGAGGCTAATGAGAAGCCAAGTACAAATCAAGAATCAAAAGTTG
TTTCACAGACTGAACAAAATTCAAAAGAAACAAAAACAGTAGAATCTAATAAGAACTTTGTTAAATTAGAT
ACTATTAAACCTGGAGCTCAAAAGATAACGGGAACTACTTTACCAAATCACTATGTTTTATTAACAGTTGA
TGGGAAAAGTGCGGATTCAGTAGAAAATGGCGGTTTGGGTTTTGTTGAAGCAAATGACAAAGGAGAATTTG
AGTACCCTTTAAATAATCGTAAAATTGTTCATAATCAAGAAATTGAGGTTTCGTCGTCAAGCCCTGATTTA
GGTGAAGATGAAGAAGATGAAGAGGTGGAAGAAGCTTCAACTGATAAAGCTGGCGTTGAGGAAGAAAGTAC
AGAAGCTAAAGTTACTTACACAACACCGCGATATGAAAAAGCGTATGAAATACCGAAAGAACAACTAAAAG
AAAAAGATGGACATCACCAAGTTTTTATCGAACCTATTACTGAAGGATCAGGTATTATTAAAGGGCATACG
TCTGTAAAAGGTAAAGTTGCTTTATCTATTAATAATAAATTTATTAATTTTGAAGAGAGCGTTAAGGGCGG
AGTTAGTAAAGAAGACACTAAAGCTAGTTCAGATGGTATCTGGATGCCTATTGATGACAAAGGATACTTTA
ACTTTGACTTCAAAACGAAACGTTTCGATAATTTAGAGTTAAAAGAAGGTAATGACATTTCACTAACATTT
GCACCTGATGATGAAGAAGATGCATTAAAACCTTTAATTTTCAAAACTAAAGTAACGAGCTTAGAAGATAT
CGATAAAGCAGAAACTAAATATGACCATACTAAACTCAACAAAGTGAAAGTTTTAGATAATGTTAAAGAAG
ATTTACATGTTGATGAAATATATGGAAGCTTATATCATACAGACAAAGGTAAAGGTATTCTTGATAAAGAA
GGTACTAAAGTAATTAAAGGAAAGACTAAATTCGCGAATGCAGTAGTGAAGGTAGACTCTGAACTAGGTGA
AGCACAATTATTCCCTGATTTACAAGTAAATGAAAAAGGTGAATTTAGCTTTGACTCACATGGTGCTGGTT
TTAGATTACAAAATGGAGAAAAATTAAACTTCACAGTGGTTGATCCTATTACAGGTGACTTGTTAAGTAAT
GAGTTTGTTTCTAAAGAGATTGATATTGAAGAAACACCTGAACAAAAAGCGGATCGTGAGTTTGACGAAAA
ACTTGAAAATACGCCTGCTTACTACAAGTTATACGGCGATAAAATAGTTGGATTCGATACTAACGATTTCC
CGATTACTTGGTTCTATCCATTGGGTGAAAAGAAAGTTGAACGTACAACACCTAAATTAGAAAAATAA
SEQ IN NO: 25
ATGTCTAAAAAGTTAAAAATTATAATTCCTATTATTATTGTCTTATTATTAATAGGTGGAATCGCATGGGG
AGTTTATGCATTTTTTGCAAACACACCGAAAAATACATACTTAAAAAGTGAACAACAAACTGCAAAAATGT
ATAAAGATTATTTTAATGACCGTTTTGAAAACGAAGTGAAGTTCCAAGAAAAGATGAAAGATAATTCATTT
TTATCTTCATTAGAATTAAGCGCAGATGCATCTGATGAAATTGTTAAAGGGCTTGGTATTCCTAAATCTGT
TGTTAATGCTTCGAAAATTAAAATGTCATATGGACATGATCCTAAAAAAGAGAAATCAATGATTAATCTTG
AACCAACAATAGCAGACTCTGCATTAGGGAAATTCCAGTTAGCTGCAGATAAAGATAAGCATTATTTCGAA
TCACCATTATTTAAAGGGAAATATAGTGTTAATAATTCTGATTTATTATCAACTTATTCAAAACTTACAGG
TGAAGATGAAGAAACAGCAAAAGAAAATGGTATTACAAACCAACAACTAAATTTAAATACTCTTTTCAGTA
ATGCTCAAGCACAACAAAGTGACTACAGCAAAATTGCCGAAAAATATTCCGAACTTATTGTCGACAAATTA
GATGACGATAATTTTGATAAAGGTAAAAAAGAAGAAATTAAGGTTAATGGTGAAAAGTACAAAGTTAGACC
TGTCACGTTAACACTTAGCAGAGCTGACACTAAAAAAATTACATTAGCTGTATTAGAAGAAGCTAAAAAGG
ATAAAGACCTTAAAAAATTAATGGAAGAACAAGGTACTACAAAAGACTTTGAAAAAGACATTAAAAAAGCA
ATTGACGATGTCAAAGAAACTAAAAAGGATGAATTTGCTAAAATTCAATCTAAAATTTATACCGAAAAACA
TACGATTGTAAAACGAGAAATTACTATTACAGACAAAGAAAATAATAAAACTAAAATCAAAGGTACTAATA
CTTTAGAAGACGATAAGTTAAAACTAGATTACGCACTTGATTTCGATCAAGATAAATACACGTATGCTGAA
GCGAAATATACAATTAAAGGCGTATCTTCTAAGGAAAAAGACAATAAATACAGTGATAAATACGAATTTGG
TAAAAAGACAGAATATGATGAATCAAAAATCAAATTAGATAACCAAGAAAAAGTAGATGGCACAAAACGTC
AAGATAAAGGTAAAATCACTGTCGCGTTAGATAAATATAGCGACGAAAATGAATTCACTTTTGAAAATAAT
ATAGATTCTGACGTAAAAAATAACACTCAGAAATCTACGTTAAATATCGGCATCAAATATGCTGAAGAACC
AATTAATTTCATTTTAAAATCTAGCACAAAATTGAAAGCAGATATTGATTTTGATGATAGTGGTGCGAAAG
ATTTCAATAGTCTATCTTCAAAAGACCGTGAAAAACTTGAAAAAGAAATCGAAAAAAATGGCGGCAAAATG
TTTGAATCAATTTTAAAAAAGGCATCTAAATAA
SEQ IN NO: 26
GTGAGGAAATTTTCAAGATATGCATTTACAAGTATGGCAACAGTAACGTTGCTGAGCTCTTTGACACCTGC
AGCACTAGCGAGTGATACGAATCACAAACCAGCAACTTCAGATATTAATTTTGAAATCACGCAAAAGAGTG
ATGCAGTTAAAGCATTAAAAGAGTTACCTAAATCTGAAAATGTGAAAAATCATTATCAAGATTACTCTGTT
ACAGATGTAAAAACAGATAAGAAAGGATTCACGCATTACACGTTACAACCGAGTGTGGATGGTGTGCATGC
GCCTGACAAAGAAGTGAAAGTGCATGCGGACAAATCGGGTAAAGTCGTTTTAATCAACGGTGATACTGATG
CGAAGAAAGTAAAGCCGACAAATAAAGTGACATTAAGCAAGGATGAAGCGGCTGACAAAGCATTTAACGCA
GTTAAGATTGATAAAAATAAAGCTAAAAACCTCCAAGATGACGTTATCAAAGAAAATAAAGTCGAAATCGA
TGGTGACAGTAATAAATACATTTACAATATTGAATTAATTACAGTAACACCAGAAATTTCACATTGGAAAG
TTAAAATTGATGCAGACACAGGAGCAGTTGTTGAAAAAACGAACTTAGTTAAAGAAGCAGCAGCAACTGGC
ACAGGTAAAGGTGTGCTTGGAGATACAAAAGATATCAATATCAATAGTATTGATGGTGGATTTAGTTTAGA
GGATTTGACGCATCAAGGTAAATTATCAGCATACAATTTTAACGATCAAACAGGTCAAGCGACATTAATTA
CTAATGAAGATGAAAACTTCGTCAAAGATGATCAACGTGCTGGTGTAGATGCGAATTATTATGCTAAACAA
ACATATGATTACTACAAAAATACATTTGGTCGTGAGTCTTACGATAACCATGGTAGTCCAATAGTCTCATT
AACACATGTAAATCATTATGGTGGACAAGATAACAGAAATAACGCTGCATGGATTGGAGACAAAATGATTT
ATGGTGATGGCGATGGCCGCACGTTTACAAATTTATCAGGTGCAAATGACGTAGTAGCACATGAGTTAACA
CATGGCGTGACACAAGAAACGGCGAATTTAGAGTATAAAGATCAATCTGGTGCGTTAAATGAAAGCTTTTC
AGATGTTTTTGGATACTTTGTAGATGATGAGGATTTCTTGATGGGTGAAGATGTTTACACACCAGGAAAAG
AGGGAGATGCTTTACGAAGCATGTCAAACCCAGAACAATTTGGTCAACCATCTCATATGAAAGACTATGTA
TACACTGAAAAAGATAACGGTGGTGTGCATACGAATTCTGGCATTCCAAATAAAGCAGCTTATAACGTAAT
TCAAGCAATAGGGAAATCTAAATCAGAACAAATTTACTACCGAGCATTAACGGAATACTTAACAAGTAATT
CAAACTTCAAAGATTGTAAAGATGCATTATACCAAGCGGCTAAAGATTTATATGACGAGCAAACAGCTGAA
CAAGTATATGAAGCATGGAACGAAGTTGGCGTCGAGTAA
SEQ IN NO: 27
ATGAAAAAGAAATTAGGTATGTTACTTCTTGTACCAGCCGTAACTTTATCATTAGCCGCATGTGGGAATGA
TGATGGAAAAGATAAAGATGGCAAGGTAACAATTAAAACGACAGTTTATCCATTGCAATCATTTGCAGAGC
AAATTGGTGGAAAACACGTGAAGGTATCATCAATCTATCCAGCAGGGACAGATTTACATAGCTATGAACCA
ACACAAAAAGATATATTAAGTGCAAGCAAGTCAGACTTGTTTATGTATACAGGGGATAATTTAGATCCGGT
TGCTAAGAAAGTTGCATCTACTATTAAAGATAAAGATAAAAAACTGTCTTTAGAAGATAAATTAGATAAAG
CAAAGCTTTTAACTGATCAACACGAACATGGTGAAGAGCATGAACATGAGGGACATGATCATGGGAAAGAA
GAACATCATCATCATGGCGGATATGATCCACACGTATGGTTAGATCCTAAAATTAACCAAACTTTCGCTAA
AGAAATTAAAGATGAATTAGTGAAGAAAGATCCAAAACATAAAGATGACTATGAGAAAAACTACAAAAAAT
TAAACGACGATCTTAAGAAAATTGATAACGATATGAAGCAAGTTACTAAAGATAAGCAAGGTAATGCAGTA
TTCATTTCACATGAATCAATTGGATACTTAGCTGATCGTTATGGTTTTGTTCAAAAAGGTATTCAAAACAT
GAATGCTGAAGATCCATCACAAAAAGAATTGACTAAAATTGTTAAAGAAATTAGAGATAGCAATGCTAAAT
ATATTCTTTACGAAGATAATGTTGCGAATAAAGTGACTGAAACAATTCGTAAAGAAACAGATGCGAAGCCT
TTAAAATTCTACAACATGGAGTCTTTAAATAAAGAACAACAGAAAAAAGATAATATTACCTATCAATCATT
AATGAAATCGAATATTGAAAATATCGGTAAAGCTTTAGACAGTGGTGTTAAAGTGAAAGACGACAAAGCTG
AAAGTAAACACGACAAAGCAATTTCTGATGGGTATTTTAAAGATGAGCAAGTTAAAGACCGTGAATTAAGC
GATTATGCTGGTGAATGGCAATCTGTTTACCCTTACTTAAAAGACGGTACGCTTGATGAAGTGATGGAACA
TAAAGCTGAAAATGATCCGAAGAAATCTGCTAAAGATTTAAAAGCTTATTATGACAAAGGATATAAAACTG
ATATTACTAACATTGATATAAAAGGAAATGAAATTACATTTACTAAAGATGGTACGAAACACACTGGTAAA
TATGAATACAATGGTAAGAAAACATTGAAATATCCTAAAGGTAACCGTGGCGTGAGATTTATGTTTAAATT
GGTCGATGGTAATGATAAAGACTTACCGAAATTCATCCAATTTAGCGATCACAACATTGCACCTAAAAAGG
CAGAACACTTCCATATCTTTATGGGTAATGATAATGACGCGTTATTAAAAGAAATGGATAACTGGCCAACA
TATTATCCTTCAAAATTAAATAAAGACCAAATCAAAGAAGAAATGTTAGCGCATTAA
SEQ IN NO: 28
ATGGTGTTATATATCATTTTGGCAATAATTGTGATTATATTGATTGCTGTAGGTGTATTATTCTATTTACG
TTCAAATAAAAGACAAATAATAGAAAAAGCAATCGAACGTAAAAATGAAATTGAAACGTTACCTTTTGATC
AAAACCTTGCACAATTATCTAAGTTGAATTTAAAAGGTGAAACAAAAACGAAATACGATGCAATGAAAAAG
GACAACGTAGAAAGTACAAATAAGTATCTAGCTCCTGTGGAAGAAAAAATCCATAATGCTGAGGCTTTATT
AGATAAATTTAGTTTCAACGCATCTCAATGTGAAATTGATGATGCAAATGAGTTGATGGATAGTTACGAAC
AAAGCTATCAGCAACAATTAGAAGATGTAAATGAAATTATTGCGTTATACAAAGATAATGATGAATTATAT
GACAAATGTAAGGTTGATTATCGTGAAATGAAACGTGATGTTTTAGCAAATCGTCATCAATTTGGTGAGGC
AGCAAGTCTTCTTGAAACTGAAATTGAAAAATTCGAGCCAAGGTTAGAGCAATATGAAGTACTAAAAGCTG
ATGGTAATTATGTACAAGCGCACAACCATATAGCTGCCTTGAATGAACAAATGAAACAGCTAAGATCTTAT
ATGGAAGAAATACCAGAATTAATTAGAGAAACTCAAAAAGAATTACCTGGTCAATTCCAAGATTTAAAATA
TGGTTGCCGTGATCTTAAAGTTGAAGGGTATGATCTGGATCACGTAAAAGTAGACAGTACATTACAAAGCT
TAAAAACAGAGCTTAGTTTCGTTGAACCATTAATTAGTCGCTTAGAATTAGAAGAAGCTAATGATAAACTA
GCTAATATCAATGATAAGTTAGATGACATGTATGATTTAATTGAACATGAAGTTAAAGCTAAAAATGATGT
CGAAGAAACAAAAGATATCATTACGGATAACTTATTCAAAGCAAAAGACATGAATTATACATTGCAAACAG
AAATTGAATATGTACGTGAAAACTACTATATAAATGAATCTGATGCTCAGAGTGTTCGTCAATTTGAAAAT
GAAATTCAAAGTTTAATTTCTGTATATGATGATATTTTAAAAGAAATGTCTAAATCTGCTGTACGATATAG
CGAGGTTCAGGATAATTTACAATATTTAGAAGATCATGTCACAGTTATTAATGACAAACAAGAAAAGCTAC
AAAATCATCTGATTCAATTGCGTGAAGATGAAGCAGAAGCAGAAGACAATCTGTTACGAGTACAATCGAAG
AAAGAAGAAGTGTATCGTCGATTACTTGCTTCTAACTTAACAAGCGTTCCTGAAAGGTTTATCATCATGAA
AAATGAAATTGATCATGAAGTTCGTGATGTTAACGAACAATTTAGTGAACGTCCAATACACGTTAAACAGT
TAAAAGATAAAGTGTCTAAAATTGTGATTCAAATGAATACATTTGAAGATGAAGCAAATGATGTTCTTGTT
AATGCTGTTTATGCAGAGAAATTAATTCAATATGGAAATAGATATCGTAAGGACTATAGCAATGTTGATAA
GAGCTTAAATGAAGCTGAACGATTATTTAAAAATAATCGCTATAAGCGTGCGATTGAAATTGCAGAGCAAG
CTCTTGAAAGTGTTGAGCCAGGTGTCACTAAACATATTGAAGAAGAAGTTATTAAGCAATAG
SEQ IN NO: 29
ATGCCTAAAAATAAAATTTTAATTTATTTGCTATCAACTACGCTCGTATTACCTACTTTAGTTTCACCTAC
CGCTTATGCTGACACACCTCAAAAAGATACTACAGCTAAGACAACATCTCATGATTCCAAAAAATCTACTG
ATGATGAAACTTCTAAGGATACTACAAGTAAAGATATTGATAAAGCAGACAACAATAATACTAGTAACCAA
GACAATAACGACAAAAAAGTTAAAACTATAGACGACAGCACTTCAGACTCTAACAATATCATTGATTTTAT
TTATAAGAATTTACCACAAACCAATATAAACCAATTGCTAACCAAAAATAAATACGATGATAATTACTCAT
TAACAACTTTAATCCAAAACTTATTCAATTTAAATTCGGATATTTCTGATTACGAACAACCTCGTAATGGT
GAAAAGTCAACAAATGATTCGAATAAAAACAGTGATAATAGCATCAAAAATGATACGGATACGCAATCATC
TAAACAAGATAAAGCAGACAATCAAAAAGCACCTAAATCAAACAATACAAAACCAAGTACATCTAATAAGC
AACCAAATTCGCCAAAGCCAACACAACCAAATCAATCAAATAGTCAACCAGCAAGTGACGATAAAGTAAAT
CAAAAATCTTCATCGAAAGATAATCAATCAATGTCAGATTCGGCTTTAGATTCTATTTTGGATCAATACAG
TGAAGATGCAAAGAAAACACAAAAAGATTACGCATCTCAATCTAAAAAAGACAAAAATGAAAAATCTAATA
CAAAGAATCCACAGTTACCAACACAAGATGAATTGAAACATAAATCTAAACCTGCTCAATCATTCAATAAC
GATGTTAATCAAAAGGATACACGTGCAACATCACTATTCGAAACAGATCCTAGTATATCTAACAATGATGA
TAGTGGACAATTTAACGTTGTTGACTCAAAAGATACACGTCAATTTGTCAAATCAATTGCTAAAGATGCAC
ACCGCATTGGTCAAGATAACGATATTTATGCGTCTGTCATGATTGCCCAAGCAATCTTAGAATCTGACTCA
GGTCGTAGTGCTTTAGCTAAGTCACCAAACCATAATTTATTCGGTATCAAAGGTGCTTTTGAAGGGAATTC
TGTTCCTTTTAACACATTAGAAGCTGATGGTAATCAATTGTATAGTATTAATGCTGGATTCCGAAAATATC
CAAGCACGAAAGAATCACTAAAAGATTACTCTGACCTTATTAAAAATGGTATTGATGGCAATCGAACAATT
TATAAACCAACATGGAAATCGGAAGCCGATTCTTATAAAGATGCAACATCACACTTATCTAAAACATATGC
TACAGATCCAAACTATGCTAAGAAATTAAACAGTATTATTAAACACTATCAATTAACTCAGTTTGACGATG
AACGTATGCCAGATTTAGATAAATATGAACGTTCTATCAAGGATTATGATGATTCATCAGATGAATTCAAA
CCTTTCCGCGAGGTATCTGATAATATGCCATATCCACATGGCCAATGTACTTGGTACGTATATAACCGTAT
GAAACAATTTGGTACATCTATCTCAGGTGATTTAGGTGATGCACATAATTGGAATAATCGAGCTCAATACC
GTGATTATCAAGTAAGTCATACACCAAAACGTCATGCTGCTGTTGTATTTGAGGCTGGACAATTTGGTGCA
GATCAACATTACGGTCATGTAGCATTTGTTGAAAAAGTTAACAGTGATGGTTCTATCGTTATTTCAGAATC
CAATGTTAAAGGATTAGGTATCATTTCTCATAGAACTATCAATGCAGCTGCCGCTGAAGAATTATCATATA
TTACAGGTAAATAA
SEQ IN NO: 30
ATGATGAAAAGTCAAAATAAGTATAGTATTCGTAAATTTAGTGTAGGTGCATCTTCCATTTTAATAGCTAC
ATTACTATTTTTAAGTGGTGGACAAGCACAAGCAGCTGAGAAGCAAGTGAATATGGGAAATTCACAGGAGG
ATACAGTTACAGCACAATCTATTGGGGATCAACAAACTAGGGAAAATGCTAATTATCAACGTGAAAACGGT
GTTGACGAACAGCAACATACTGAAAATTTAACTAAGAACTTGCATAATGATAAAACAATATCAGAAGAAAA
TCATCGTAAAACAGATGATTTGAATAAAGATCAACTAAAGGATGATAAAAAATCATCGCTTAATAATAAAA
ATATTCAACGTGATACAACAAAAAATAACAATGCTAATCCTAGGGATGTAAATCAAGGGTTAGAACAGGCT
ATTAATGATGGCAAACAAAGTAAAGTGGCGTCACAGCAACAGTCAAAAGAGGCAGATAATAGTCAAGACTT
AAACGCTAATAACAATCTACCTTCACAAAGTCGAACAAAGGTATCACCATCATTAAATAAGTCAGATCAAA
CAAGTCAACGAGAAATTGTTAATGAGACAGAAATAGAGAAAGTACAACCGCAACAAAAGAATCAAGCGAAT
GATAAAATTACTGACCACAATTTTAACAATGAACAAGAAGTGAAACCTCAAAAAGACGAAAAAACACTATC
AGTTTCAGATTTAAAAAACAATCAAAAATCACCAGTTGAACCAACAAAGGACAATGACAAGAAAAATGGAT
TAAATTTATTAAAAAGTAGTGCAGTAGCAACGTTACCAAACAAAGGGACAAAGGAACTTACTGCAAAAGCG
AAAGGTGATCAAACGAATAAAGTTGCCAAACAAGGGCAGTATAAAAATCAAGATCCTATAGTTTTAGTGCA
TGGTTTCAATGGGTTTACAGATGATATTAATCCTTCAGTGTTAGCTCATTATTGGGGCGGTAATAAAATGA
ACATTCGCCAAGATTTAGAAGAAAATGGTTACAAAGCTTATGAAGCAAGTATAAGTGCTTTTGGAAGTAAC
TATGACCGCGCAGTTGAACTTTATTATTATATCAAAGGCGGTCGTGTAGATTATGGTGCAGCACATGCAGC
AAAATATGGACATGAACGTTATGGAAAAACATACGAAGGAATTTACAAAGACTGGAAACCAGGACAGAAGG
TACACCTTGTTGGACATAGTATGGGTGGTCAAACGATACGTCAACTAGAAGAATTACTGCGTAATGGTAGT
CGTGAAGAAATAGAGTATCAAAAGAAACATAGTGGCGAAATTTCTCCACTATTCAAAGGTAATAATGACAA
TATGATTTCATCAATTACTACTTTAGGAACGCCACATAATGGAACGCATGCTTCAGATTTAGCTGGTAATG
AAGCTTTAGTGAGACAAATTGTATTTGATATCGGTAAAATGTTTGGTAATAAAAATTCAAGAGTAGACTTC
GGGTTGGCTCAATGGGGTCTAAAACAGAAGCCAAATGAATCATATATTGATTATGTCAAACGCGTTAAACA
ATCTAATTTATGGAAATCAAAAGATAATGGATTTTACGATCTGACGCGTGAGGGTGCAACAGATTTAAATC
GTAAAACGTCGTTGAACCCTAACATTGTGTATAAAACATACACTGGTGAAGCAACGCACAAAGCATTAAAT
AGCGATAGACAAAAAGCAGACTTAAATATGTTTTTCCCATTTGTGATTACTGGTAACTTAATCGGTAAAGC
TACTGAAAAAGAATGGCGAGAAAACGATGGTTTAGTATCCGTTATTTCTTCTCAACATCCATTTAATCAAG
CTTATACAAATGCGACGGATAAAATTCAAAAAGGCATTTGGCAAGTAACGCCTACAAAACATGATTGGGAT
CATGTTGATTTTGTCGGACAAGATAGTTCTGATACAGTGCGCACAAGAGAAGAATTACAAGATTTTTGGCA
TCATTTAGCAGACGATTTAGTGAAAACTGAAAAGGTGACTGATACTAAGCAAGCATAA
SEQ IN NO: 31
ATGACAAATAAAATGAAGAAATGGCAAAAATTATCCACCATTACGTTATTAATGACCGGAGTGATTGCTTT
AAATAATGGTGAATTTAGAAATGTTGATAAACATCAAATCGCTGTGGCTGATACGAATGTTCAAACGCCAG
ATTATGAAAAATTGAAGAAGACGTGGCTCGACGTTAACTACGGTTATGATCAGTATGATGAGAATAATCAA
GATATGAAGAAGAAGTTTGATGCTAAAGAAAAAGAAGCCAAGAAGTTACTTGATGACATGAAAACTGATAC
GAATAGAACATATTTGTGGTCAGGAGCTGAAAACCTTGAAACTAATTCTTCTCACATGACAAAAACCTATC
GTAATATCGAGAAAATCGCAGAATCAATGCAACATAAGAATACGGTATTAAAAACAGTTGAAAACAAGTTG
AAAATAAAAGAAGCCCTAGATTGGATGCACAAAAATGTTTATGGCAAGAATCCTTCTCAAAAAGTCGAGGA
TTTAACTAAAAATCGTAAGGGGCAAACTACACCCAAGAATAACTCATTGAATTGGTGGGATTATGAAATTG
GTACGCCAAGAGCATTAACAAATACACTACTTCTAATGGATGATATGCTCACTAAAGATGAAATGAAAAAT
TATTCAAAACCTATTAGTACATATGCACCATCCAGTGACAAAATTTTATCTTCTGTTGGTGAATCAGAAGA
TGCTAAAGGTGGAAATTTAGTGGACATTTCTAAAGTAAAACTTTTAGAAAGTGTTATTGAAGAAGATGTAG
ATATGTTGAAAAAGTCTATAGATTCTTTTAATAAAGTGTTCACTTATGTTCAAGATTCTGCCACTGGTAAA
GGTCGCAATGGATTCTATAAAGATGGCTCTTACATTGATCATCAAGATGTCCCTTACACTGGTGCTTATGG
TGTTGTACTATTAGAGGGTATTTCTCAAATGATGCCGATGATAAAAGAATCTCCTTTTAAAACTACACAAG
ATAATGCTACATTAAGCAATTGGATTGACGAAGGGTTTATGCCATTAATCTATAAAGGTGAAATGATGGAT
TTATCACGAGGTAGAGCTATCAGTCGTGAAAATGAAACGAGTCATACAGCGTCAGCGACTGTAATGAAATC
ATTGTTGAGATTGAATGATACCATGGATGATTCAACAAAAACTAGATATAAGCAAATCGTTAAAACTTCTG
TTAATTCTGATTCAAGTTACAACCAAAATAATTATTTAAATTCATATTCAGACATAGCTAAAATGAAAAAG
TTAATGAATGATAGTACTATTTCTAAAAACGATTTAACACAGCAACTTAAAATATATAATGACATGGATCG
TGTCACCTATCACAATAAAGACCTGGACTTTGCATTTGGTTTAAGTATGACATCGAAAAACATCGCACGAT
ACGAAAATATCAACGGAGAGAACTTAAAAGGTTGGCACACCGGTGCAGGCATGTCTTATTTATATAACAGC
GATGTCAAACACTATCGCGATAACTTCTGGGCAACAGCCGATATGACTTGTCTTCCAGGCACTACTACTTT
AAATGATATGCCATCTACTAATACTAAGAATGATAAATCTTTTGTTGGCGGGACAAAATTAAATAATAAAT
ACGCAAGCATCGGTATGGATTTTGAAAATCAGGACAAAACTTTAACTGCCAAAAAATCATATTTCATATTA
AACGATAAAATTGTCTTCTTAGGAACTGGCATTAAAAGTACTGATTCATCAAAGAATCCAGTTACAAGTGT
TGAAAATCGCAAAGCAAATGGGTATAAATTATTTAAAGATGATATTGAAATTACCACTTCAGATGTTAATG
CTCAGGAAACCCATTCAGTCTTTTTAGAGTCCAACGATACTAAAAAGAACATTGGTTATCATTTCTTAGAC
AAGCCAAAAATAACTGTAAAAAAAGAAAGTCATACTGGTAAGTGGAGTGAAATTAATAAAAGTCAAAAAAA
AGATGACAAAAAAGATGAGTATTATGAAGTAACTCAAACACATAATACATCTGACAGTAAATATGCATATG
TTTTGTATCCTGGTTTATCAAAAAGTGATTTTAAATCGAAGAATAATAATGTAAGTATTGTTAAACAAGAT
GAAGATTTTCATGTGATAAAAGATAATGATGGCGTATTTGCTGGGGTTAATTATAGTGATAATACTAAATC
TTTTGATATAAACGGAATTACTGTTGAATTAAAAGAAAAAGGCATGTTTGTAATTAAAAAGAAAGATGATA
AAGCATATAAATGTAGCTTCTATAATCCTGAAACTACAAATACCGCTTCAAATATAGAATCAAAAATTTTT
ATTAAAGGTTACACCATAACTAATAAAAGTGTCATAAACTCTAATGATGCTGGTGTAAACTTTGAATTAAC
TAAATAA
SEQ IN NO: 32
ATGACATATAGAATGAAGAAATGGCAAAAATTGTCCACCATTACGTTATTAATGGCTGGTGTGATTACTTT
GAATGGTGGTGAATTCAGAAGTATTGATAAACATCAAATCGCTGTGGCTGATACGAATGTTCAAACGACAG
ATTATGAAAAGTTGAGGAACATATGGCTGGACGTTAACTATGGTTATGATAAGTATGATGAGAATAATCCA
GATATGAAGAAGAAGTTTGAGGCTACGGAGAATGAGGCAGAGAAATTACTCAAGGAAATGAAAACTGAAAG
TGATAGGAAATACTTGTGGGAAAGCTCAAAAGATTTAGATACGAAGTCTGCGGATATGACTCGTACCTATC
GTAATATTGAGAAAATCTCAGAAGCGATGAAACATAAAAATACTAAATTAAAAACAGATGAAAACAAGACA
AAAGTAAAAGATGCACTTGAGTGGCTGCATAAAAATGCATATGGAAAAGAACCAGATAAAAAAGTTGCTGA
TTTGACCTCAAACTTTAAAAATAAAACTTCTAGAAATACCAACTTAAATTGGTGGGATTATGAAATTGGAA
CACCTAGAGCATTAACAAATACGCTTATACTCTTACAAGAAGATTTCACTGATGAAGAAAAGAAAAAATAT
ACAGCTCCTATTAAAACTTTCGCCCCAGATAGTGACAAAATATTATCTTCTGTAGGAAAATCTGAACCTGC
TAAAGGCGGAAATTTAGTAGACATTTCTAAAGTAAAACTTTTAGAAAGTATTATCGAAGAAGACAAAGATA
TGATGAAAAAGTCTATAGATTCATTTAATACAGTCTTCACTTACGCGCAAAATTCTGCCACTGGAAAAGAA
CGTAATGGATTCTATAAAGATGGCTCTTACATTGATCATCAAGACGTCCCATACACTGGTGCTTATGGCGT
TGTACTATTAGAGGGTATTTCTCAAATGATGCCGATGATAAAAGAAACACCTTTTAATGATAGTAACCAAA
ATGATACAACCTTAAAATCATGGATTGACGACGGATTTATGCCACTCATTTATAAAGGTGAAATGATGGAT
TTATCAAGAGGTAGAGCTATCAGTCGTGAAAATGAAACGAGTCACTCAGCATCTGCAACAGTAATGAAATC
ATTGTTGAGATTGAGTGATACCATGGATAAGTCTACAAAAGCTAAATATAAAAAGATTGTCAAGACTTCAG
TAGAGTCAGATTCAAGTTATAAACAAACCGATTATTTAAGCTCTTATTCGGATATAAGCAAAATGAAGTCT
TTAATGGAAGACAGCACTATTTCTACTAACGGTTTAACACAACAACTTAAAATATATAATGACATGGATCG
TGTCACCTATCACAATAAAGGCTTAGACTTTGCATTTGGTTTAAGTATGACGTCGAAAAACGTCGCACGTT
ACGAAAGTATCAACGGAGAGAACTTAAAAGGTTGGCACACTGGTGCTGGAATGTCTTATTTATACAATAGC
GATGTGAAACACTACCGTGATAACTTCTGGGCGACAGCTGATATGAAACGTTTAGCAGGTACTACAACTTT
AGATAATGAAGAACCTAAAAGTACGGATGTTAAAAAGTCTAGTAAAACTTTTGTAGGAGGAACAAAATTCG
ATGACCAACATGCTAGTATCGGAATGGATTTTGAAAATCAGGACAAAACTTTAACTGCCAAAAAATCATAT
TTCATATTAAACGATAAAATTGTCTTCTTAGGAACTGGCATTAAAAGTACTGATTCATCAAAGAATCCAGT
TACAACGATTGAAAATCGCAAAGCGAATGATTATAAATTATATAAAGATGATACGCAAACAACCAATTCCG
ATAATCAGGAAACCAATTCCCTCTTTTTAGAGTCAACGAATAGCACTCAAAACAATATAGGTTATCATTTT
TTAAACGAATCGAAAATAACTGTAAAAAAAGAAAGTCATACTGGTAAGTGGAGTGATATAAATAAAAGCCA
AAAGGATATACAAAAAACTGATGAGTATTATGAAGTAACTCAAAAGCATTCTAATACAGATAGTAAATATG
CATATGTGTTGTATCCAGGCTTATCTAAAGATGTCTTTAAATCCAAAGCAAGCAAAGTAACTGTCGTTAAG
CAAGAAGATGACTTCCACGTTGTGAAAGATAATGAATCGGTTTGGGCTGGTATCAATTATAGTGATAGCGC
TAAAACTTTTGAAATTAATAACACTAAAGTCGAAGTTAAAGCCAAAGGAATGTTTATTCTTACAAAGAAAG
ATGATAACACTTATGAATGTAGCTTCTATAATCCCGAATCTACAAATTCCGTTTCAGATATTGAATCTAAA
ATTTCAATGACTGGATACTCTATTATAAACAAAAATACGTCGACTTCTAATGAATCCGGCGTACGCTTTGA
ATTAACTAAATAA
SEQ IN NO: 33
AUGGCAAAAGGUAAUUUAUUUAAAGCGAUUUUAGGUAUAGGUGGCGCUGUAGCAGCUGUACUUGUUACACG
UAAAGAUAGUCGUGACAAGCUGAAAGCAGAAUAUAAUAAAUACAAACAAGAUCCUCAAAGCUAUAAAGAUA
AUGCUAAGGAUAAAGCGACGCAAUUAGGAACAAUUGCAAAUGAAACAAUUAAAGAAGUAAAAACAAAUCCG
AAAGAAUAUGCUAAUAGAUUAAAAAAUAAUCCAAAAGCAUUUUUCGAAGAAGAAAAAUCAAAAUUUACCGA
AUAUGACAAUAAGACUGACGAAAGUAUUGAAAAAGGUAAAUUUGAUGAUGAAGGUGGCGCAGCACCAAAUA
AUAAUUUACGUAUCGUCACUGAAGAAGAUUUAAAAAAGAAUAAAAAUGCAUUGUCUGAUAAAGAAUAA
SEQ IN NO: 34
AUGAAAAAAUUGGUUUCAAUUGUUGGCGCAACAUUAUUGUUAGCUGGAUGUGGAUCACAAAAUUUAGCACC
AUUAGAAGAAAAAACAACAGAUUUAAGAGAAGAUAAUCAUCAACUCAAACUAGAUAUUCAAGAACUUAAUC
AACAAAUUAGUGAUUCUAAAUCUAAAAUUAAAGGGCUUGAAAAGGAUAAAGAAAAUAGUAAAAAAACUGCA
UCUAAUAAUACGAAAAUUAAAUUGAUGAAUGUUACAUCAACAUACUACGACAAAGUUGCUAAAGCUUUGAA
AUCCUAUAACGAUAUUGAAAAGGAUGUAAGUAAAAACAAAGGCGAUAAGAAUGUUCAAUCGAAAUUAAAUC
AAAUUUCUAAUGAUAUUCAAAGUGCUCACACUUCAUACAAAGAUGCUAUCGAUGGUUUAUCACUUAGUGAU
GAUGAUAAAAAAACGUCUAAAAAUAUCGAUAAAUUAAACUCUGAUUUGAAUCAUGCAUUUGAUGAUAUUAA
AAAUGGCUAUCAAAAUAAAGAUAAAAAACAACUUACAAAAGGACAACAAGCGUUGUCAAAAUUAAACUUAA
AUGCAAAAUCAUGA
SEQ IN NO: 35
AUGAAAAAAUUAGUUACAGGGUUAUUAGCAUUAUCAUUAUUUUUAGCUGCAUGUGGUCAAGAUAGUGACCA
ACAAAAAGACAGUAAUAAAGAAAAAGAUGAUAAAGCGAAAACUGAACAACAAGAUAAAAAAACAAAUGAUU
CAUCUAAAGAUAAGAAAGACAAUAAAGAUGAUAGUAAAGACGUAAACAAAGAUAAUAAAGAUAAUAGUGCA
AACGAUAACCAGCAACAAUCUAAUUCAAAUGCAACAAACAAUGACCAAAAUCAAACGAAUAAUAACCAGUC
AAACAGUGGACAAACGACUAACAAUCAAAAAUCAAGUUACGUUGCACCAUAUUAUGGACAAAACGCAGCGC
CAGUGGCUCGUCAAAUUUAUCCAUUUAAUGGUAAUAAAUCACAAGCAUUACAACAAUUGCCUAAUUUCCAA
ACAGCUUUAAAUGCAGCUAACAACGAAGCAAAUAAAUUUGGUAAUGGUCAUAAAGUUUAUAAUGAUUAUUC
AAUUGAAGAACAUAAUGGUAACUAUAAGUAUGUUUUUAGUUUUAAAGACCCAAACGUAAAUGGAAAAUAUU
CAAUUGUAACGGUUGAUUAUACUGGACAAGCAAUGGUUACUGAUCCAAACUACCAACAAUAA
SEQ IN NO: 36
AUGAAAAAAGUAAUGGGGAUAUUAUUAGCAAGUACACUUAUCUUAGGUGCUUGUGGACAUCAUCAAGAUAG
UGCAAAAAAAGAGAGCACUAGUCACAAAAAGAAAGAAAAUGACAAUGAAGAAUUAAAUGAAGAACUUAAAG
AAUUUAAAAGCAAAAAAAAUAUGGAUAUAAAAAUUAAAGGCGAUACUAUUGUUAGUGACAAAUUUGAAGCU
AAAAUAAAAGAACCGUUUAUCAUCAAUGAAAAAGAUGAGAAAAAGAAAUAUAUCGCUUUUAAAAUGGAAAU
UACUGCUAAAAAAGACGAUAAAGAUUUAAAUCCAUCUUCUAUUUCUCAUGACUAUAUUAAUAUCACUCAAG
AUGAUAAAAAUACAGUAAAUAAAUUAAGAGAUGGUUAUCUUUUAAGUGAUAAAAAUUAUAAAGAUUGGACA
GAACAUAACCAAGAUCAAAUUAAAAAAGGCAAAACUGCACAAGCCAUGUUUAUCUAUGAGUUAAGAGGUGA
UGGAAACAUUAAUUUAAAUGUCCAUAAAUACUCAGAAGAUAAAACAGUUGAUUCUAAAUCAUUCAAAUUUA
GUAAACUUAAAACCGAAGAUUUUUCUCAUAGAGCGGAAACAAGGGAAGAAGUAGAAAAGAAAGAAAAAGAA
UUUGAAGAAGAGUACAAAAAAGAACAAGAACGAGAGAAAGAAAAAGAAAAGCAAAAAGAUGACGACCACAG
UGGUUUAGAUGAAGUAUAA
SEQ IN NO: 37
AUGAAAAAAUGGCAAUUUGUUGGUACUACAGCUUUAGGUGCAACACUAUUAUUAGGUGCUUGUGGUGGCGG
UAAUGGUGGCAGUGGUAAUAGUGAUUUAAAAGGGGAAGCUAAAGGGGAUGGCUCAUCAACAGUAGCACCAA
UUGUGGAGAAAUUAAAUGAAAAAUGGGCUCAAGAUCACUCGGAUGCUAAAAUCUCAGCAGGACAAGCUGGU
ACAGGUGCUGGUUUCCAAAAAUUCAUUGCAGGAGAUAUCGACUUCGCUGAUGCUUCUAGACCAAUUAAAGA
UGAAGAGAAGCAAAAAUUACAAGAUAAGAAUAUCAAAUACAAAGAAUUCAAAAUUGCGCAAGAUGGUGUAA
CGGUUGCUGUAAAUAAAGAAAAUGAUUUUGUAGAUGAAUUAGACAAACAGCAAUUAAAAGCAAUUUAUUCU
GGAAAAGCUAAAACAUGGAAAGAUGUUAAUAGUAAAUGGCCAGAUAAAAAAAUAAAUGCUGUAUCACCAAA
CUCAAGUCAUGGUACUUAUGACUUCUUUGAAAAUGAAGUAAUGAAUAAAGAAGAUAUUAAAGCAGAAAAAA
AUGCUGAUACAAAUGCUAUCGUUUCUUCUGUAACGAAAAACAAAGAGGGAAUCGGAUACUUUGGAUAUAAC
UUCUACGUACAAAAUAAAGAUAAAUUAAAAGAAGUUAAAAUCAAAGAUGAAAAUGGUAAAGCAACAGAGCC
UACGAAAAAAACAAUUCAAGAUAACUCUUAUGCAUUAAGUAGACCAUUAUUCAUUUAUGUAAAUGAAAAAG
CAUUGAAAGAUAAUAAAGUAAUGUCAGAAUUUAUCAAAUUCGUCUUAGAAGAUAAAGGUAAAGCAGCUGAA
GAAGGUGGAUAUGUAGCAGCACCAGAGAAAACAUACAAAUCACAAUUAGAUGAUUUAAAAGCAUUUAUUGA
UAAAAAUCAAAAAUCAGACGACAAGAAAUCUGAUGAUAAAAAGUCUGAAGACAAGAAAUAA
SEQ IN NO: 38
AUGAAAGGUAAAUUUUUAAAAGUUAGUUCUUUAUUCGUUGCAACUUUGACAACAGCGACACUUGUGAGUUC
UCCAGCAGCAAAUGCGUUAUCUUCAAAAGCUAUGGACAAUCAUCCACAACAAACGCAGACAGACAAACAGC
AAACACCUAAGAUUCAAAAAGGCGGUAACCUUAAACCAUUAGAACAACGUGAACGCGCUAAUGUUAUAUUA
CCAAAUAACGAUCGUCACCAAAUCACAGAUACAACGAAUGGUCAUUAUGCACCUGUUACUUAUAUUCAAGU
UGAAGCACCUACUGGUACAUUUAUUGCUUCUGGUGUAGUUGUAGGUAAAGAUACACUUUUAACAAAUAAAC
ACAUCGUAGAUGCUACGCACGGUGAUCCUCAUGCUUUAAAAGCAUUCGCUUCUGCAAUUAACCAAGACAAU
UAUCCUAAUGGUGGUUUCACUGCUGAACAAAUCACUAAAUAUUCAGGCGAAGGUGAUUUAGCAAUCGUUAA
AUUCUCCCCUAAUGAGCAAAACAAACAUAUUGGCGAAGUAGUUAAACCAGCAACAAUGAGUAAUAAUGCUG
AAACACAAGUUAACCAAAAUAUUACUGUAACAGGAUAUCCUGGUGAUAAACCUGUCGCAACAAUGUGGGAA
AGUAAAGGAAAAAUAACGUACUUAAAAGGUGAAGCAAUGCAAUAUGAUUUAAGUACAACUGGUGGUAACUC
AGGUUCACCUGUAUUUAAUGAAAAAAAUGAAGUCAUUGGCAUUCAUUGGGGUGGCGUUCCAAAUCAAUUUA
ACGGUGCAGUAUUUAUUAAUGAAAAUGUACGCAACUUCUUAAAACAAAAUAUUGAAGAUAUCAAUUUCGCA
AAUGAUGACCACCCUAACAACCCUGAUAAUCCAGACAAUCCAAAUAAUCCGGACAAUCCUAACAACCCUGA
UAACCCUAACAACCCUGAUAAUCCAGACAAUCCUAAUAAUCCUGAUAACCCUAACAACCCGGACAAUCCAA
AUAACCCUGACCAACCUAACAACCCAAAUAACCCGGACAAUGGCGAUAACAAUAAUUCAGACAACCCUGAC
GCUGCAUAA
SEQ IN NO: 39
AUGAAGCGUACAUUAGUAUUAUUGAUUACAGCUAUCUUUAUACUCGCUGCUUGUGGUAACCAUAAGGAUGA
CCAGGCUGGAAAAGAUAAUCAAAAACAUAACAAUAGUUCAAAUCAAGUAAAAGAAAUUGCUACGGAUAAAA
AUGUACAAGGUGAUAACUAUCGUACAUUGUUACCAUUUAAAGAAAGCCAGGCAAGAGGACUUUUACAAGAU
AACAUGGCAAAUAGUUAUAAUGGCGGCGACUUUGAAGAUGGUUUAUUGAACUUAAGUAAAGAAGUGUUUCC
AACAGACAAAUAUUUGUAUCAAGAUGGUCAAUUUUUGGACAAGAAAACAAUUAAUGCCUAUUUAAAUCUUA
AGUAUACAAAACGUGAAAUCGAUAAAAUGUCUGAAAAAGAUAAAAAAGACAAGAAAGCGAAUGAAAAUUUA
GGACUUAAUCCAUCACACGAAGGUGAAACAGAUCCUGAAAAGAUUGCAGAAAAAUCACCAGCCUAUUUAUC
UAACAUUUUAGAGCAAGAUUUUUAUGGUGGUGGAGAUACAAAAGGUAAGAAUAUUAAAGGUAUGACGAUUG
GUUUAGCUAUGAAUAGUGUUUAUUACUAUAAAAAAGAAAAAGAUGGACCGACUUUUAGUAAAAAACUAGAU
GAUAGCGAAGUUAAAAAGCAAGGUAAACAAAUGGCUAGUGAGAUAUUAUCAAGGUUACGUGAAAAUGAUGA
UUUAAAAGAUAUACCAAUUCAUUUUGCAAUUUAUAAGCAAUCAAGUGAAGAUUCAAUCACACCAGGUGAAU
UUAUCACUCAAGCGACUGCAGAAAAGAGUCAAACAAAGCUUAAUGAAUGGCAUAAUAUCAAUGAAAAAUCA
GCUUUAUUACCUUCUUCAACAGCAGCAGAUUAUGAUGAAAAUUUAAAUAAUAAUUUCAAGCAAUUUAAUGA
UAAUUUGCAAUCAUAUUUUUCUAAUUUCACACAAGCAGUAGGAAAAGUUAAAUUUGUUGAUAAAAAGCCAC
AACGAUUAGUAGUAGAUUUACCAAUCGAUUACUAUGGACAAGCUGAAACAAUUGGUAUUACACAGUACGUU
ACUGAACAAGCGAAUAAAUAUUUCGAUAAAAUCGAUAACUAUGAAAUUCGGAUUAAAGAUGGUAACCAACC
ACGUGCUUUAAUUAGUAAGACAAAAGAUGACAAAGAACCGCAAGUUCAUAUUUACAGUAAUUAA
SEQ IN NO: 40
AUGAGGGAAAAUUUUAAGUUACGUAAAAUGAAAGUCGGGUUAGUAUCUGUUGCAAUUACAAUGUUAUAUAU
CAUGACAAACGGACAAGCAGAAGCAUCAGAGGCUAAUGAGAAGCCAAGUACAAAUCAAGAAUCAAAAGUUG
UUUCACAGACUGAACAAAAUUCAAAAGAAACAAAAACAGUAGAAUCUAAUAAGAACUUUGUUAAAUUAGAU
ACUAUUAAACCUGGAGCUCAAAAGAUAACGGGAACUACUUUACCAAAUCACUAUGUUUUAUUAACAGUUGA
UGGGAAAAGUGCGGAUUCAGUAGAAAAUGGCGGUUUGGGUUUUGUUGAAGCAAAUGACAAAGGAGAAUUUG
AGUACCCUUUAAAUAAUCGUAAAAUUGUUCAUAAUCAAGAAAUUGAGGUUUCGUCGUCAAGCCCUGAUUUA
GGUGAAGAUGAAGAAGAUGAAGAGGUGGAAGAAGCUUCAACUGAUAAAGCUGGCGUUGAGGAAGAAAGUAC
AGAAGCUAAAGUUACUUACACAACACCGCGAUAUGAAAAAGCGUAUGAAAUACCGAAAGAACAACUAAAAG
AAAAAGAUGGACAUCACCAAGUUUUUAUCGAACCUAUUACUGAAGGAUCAGGUAUUAUUAAAGGGCAUACG
UCUGUAAAAGGUAAAGUUGCUUUAUCUAUUAAUAAUAAAUUUAUUAAUUUUGAAGAGAGCGUUAAGGGCGG
AGUUAGUAAAGAAGACACUAAAGCUAGUUCAGAUGGUAUCUGGAUGCCUAUUGAUGACAAAGGAUACUUUA
ACUUUGACUUCAAAACGAAACGUUUCGAUAAUUUAGAGUUAAAAGAAGGUAAUGACAUUUCACUAACAUUU
GCACCUGAUGAUGAAGAAGAUGCAUUAAAACCUUUAAUUUUCAAAACUAAAGUAACGAGCUUAGAAGAUAU
CGAUAAAGCAGAAACUAAAUAUGACCAUACUAAACUCAACAAAGUGAAAGUUUUAGAUAAUGUUAAAGAAG
AUUUACAUGUUGAUGAAAUAUAUGGAAGCUUAUAUCAUACAGACAAAGGUAAAGGUAUUCUUGAUAAAGAA
GGUACUAAAGUAAUUAAAGGAAAGACUAAAUUCGCGAAUGCAGUAGUGAAGGUAGACUCUGAACUAGGUGA
AGCACAAUUAUUCCCUGAUUUACAAGUAAAUGAAAAAGGUGAAUUUAGCUUUGACUCACAUGGUGCUGGUU
UUAGAUUACAAAAUGGAGAAAAAUUAAACUUCACAGUGGUUGAUCCUAUUACAGGUGACUUGUUAAGUAAU
GAGUUUGUUUCUAAAGAGAUUGAUAUUGAAGAAACACCUGAACAAAAAGCGGAUCGUGAGUUUGACGAAAA
ACUUGAAAAUACGCCUGCUUACUACAAGUUAUACGGCGAUAAAAUAGUUGGAUUCGAUACUAACGAUUUCC
CGAUUACUUGGUUCUAUCCAUUGGGUGAAAAGAAAGUUGAACGUACAACACCUAAAUUAGAAAAAUAA
SEQ IN NO: 41
AUGUCUAAAAAGUUAAAAAUUAUAAUUCCUAUUAUUAUUGUCUUAUUAUUAAUAGGUGGAAUCGCAUGGGG
AGUUUAUGCAUUUUUUGCAAACACACCGAAAAAUACAUACUUAAAAAGUGAACAACAAACUGCAAAAAUGU
AUAAAGAUUAUUUUAAUGACCGUUUUGAAAACGAAGUGAAGUUCCAAGAAAAGAUGAAAGAUAAUUCAUUU
UUAUCUUCAUUAGAAUUAAGCGCAGAUGCAUCUGAUGAAAUUGUUAAAGGGCUUGGUAUUCCUAAAUCUGU
UGUUAAUGCUUCGAAAAUUAAAAUGUCAUAUGGACAUGAUCCUAAAAAAGAGAAAUCAAUGAUUAAUCUUG
AACCAACAAUAGCAGACUCUGCAUUAGGGAAAUUCCAGUUAGCUGCAGAUAAAGAUAAGCAUUAUUUCGAA
UCACCAUUAUUUAAAGGGAAAUAUAGUGUUAAUAAUUCUGAUUUAUUAUCAACUUAUUCAAAACUUACAGG
UGAAGAUGAAGAAACAGCAAAAGAAAAUGGUAUUACAAACCAACAACUAAAUUUAAAUACUCUUUUCAGUA
AUGCUCAAGCACAACAAAGUGACUACAGCAAAAUUGCCGAAAAAUAUUCCGAACUUAUUGUCGACAAAUUA
GAUGACGAUAAUUUUGAUAAAGGUAAAAAAGAAGAAAUUAAGGUUAAUGGUGAAAAGUACAAAGUUAGACC
UGUCACGUUAACACUUAGCAGAGCUGACACUAAAAAAAUUACAUUAGCUGUAUUAGAAGAAGCUAAAAAGG
AUAAAGACCUUAAAAAAUUAAUGGAAGAACAAGGUACUACAAAAGACUUUGAAAAAGACAUUAAAAAAGCA
AUUGACGAUGUCAAAGAAACUAAAAAGGAUGAAUUUGCUAAAAUUCAAUCUAAAAUUUAUACCGAAAAACA
UACGAUUGUAAAACGAGAAAUUACUAUUACAGACAAAGAAAAUAAUAAAACUAAAAUCAAAGGUACUAAUA
CUUUAGAAGACGAUAAGUUAAAACUAGAUUACGCACUUGAUUUCGAUCAAGAUAAAUACACGUAUGCUGAA
GCGAAAUAUACAAUUAAAGGCGUAUCUUCUAAGGAAAAAGACAAUAAAUACAGUGAUAAAUACGAAUUUGG
UAAAAAGACAGAAUAUGAUGAAUCAAAAAUCAAAUUAGAUAACCAAGAAAAAGUAGAUGGCACAAAACGUC
AAGAUAAAGGUAAAAUCACUGUCGCGUUAGAUAAAUAUAGCGACGAAAAUGAAUUCACUUUUGAAAAUAAU
AUAGAUUCUGACGUAAAAAAUAACACUCAGAAAUCUACGUUAAAUAUCGGCAUCAAAUAUGCUGAAGAACC
AAUUAAUUUCAUUUUAAAAUCUAGCACAAAAUUGAAAGCAGAUAUUGAUUUUGAUGAUAGUGGUGCGAAAG
AUUUCAAUAGUCUAUCUUCAAAAGACCGUGAAAAACUUGAAAAAGAAAUCGAAAAAAAUGGCGGCAAAAUG
UUUGAAUCAAUUUUAAAAAAGGCAUCUAAAUAA
SEQ IN NO: 42
GUGAGGAAAUUUUCAAGAUAUGCAUUUACAAGUAUGGCAACAGUAACGUUGCUGAGCUCUUUGACACCUGC
AGCACUAGCGAGUGAUACGAAUCACAAACCAGCAACUUCAGAUAUUAAUUUUGAAAUCACGCAAAAGAGUG
AUGCAGUUAAAGCAUUAAAAGAGUUACCUAAAUCUGAAAAUGUGAAAAAUCAUUAUCAAGAUUACUCUGUU
ACAGAUGUAAAAACAGAUAAGAAAGGAUUCACGCAUUACACGUUACAACCGAGUGUGGAUGGUGUGCAUGC
GCCUGACAAAGAAGUGAAAGUGCAUGCGGACAAAUCGGGUAAAGUCGUUUUAAUCAACGGUGAUACUGAUG
CGAAGAAAGUAAAGCCGACAAAUAAAGUGACAUUAAGCAAGGAUGAAGCGGCUGACAAAGCAUUUAACGCA
GUUAAGAUUGAUAAAAAUAAAGCUAAAAACCUCCAAGAUGACGUUAUCAAAGAAAAUAAAGUCGAAAUCGA
UGGUGACAGUAAUAAAUACAUUUACAAUAUUGAAUUAAUUACAGUAACACCAGAAAUUUCACAUUGGAAAG
UUAAAAUUGAUGCAGACACAGGAGCAGUUGUUGAAAAAACGAACUUAGUUAAAGAAGCAGCAGCAACUGGC
ACAGGUAAAGGUGUGCUUGGAGAUACAAAAGAUAUCAAUAUCAAUAGUAUUGAUGGUGGAUUUAGUUUAGA
GGAUUUGACGCAUCAAGGUAAAUUAUCAGCAUACAAUUUUAACGAUCAAACAGGUCAAGCGACAUUAAUUA
CUAAUGAAGAUGAAAACUUCGUCAAAGAUGAUCAACGUGCUGGUGUAGAUGCGAAUUAUUAUGCUAAACAA
ACAUAUGAUUACUACAAAAAUACAUUUGGUCGUGAGUCUUACGAUAACCAUGGUAGUCCAAUAGUCUCAUU
AACACAUGUAAAUCAUUAUGGUGGACAAGAUAACAGAAAUAACGCUGCAUGGAUUGGAGACAAAAUGAUUU
AUGGUGAUGGCGAUGGCCGCACGUUUACAAAUUUAUCAGGUGCAAAUGACGUAGUAGCACAUGAGUUAACA
CAUGGCGUGACACAAGAAACGGCGAAUUUAGAGUAUAAAGAUCAAUCUGGUGCGUUAAAUGAAAGCUUUUC
AGAUGUUUUUGGAUACUUUGUAGAUGAUGAGGAUUUCUUGAUGGGUGAAGAUGUUUACACACCAGGAAAAG
AGGGAGAUGCUUUACGAAGCAUGUCAAACCCAGAACAAUUUGGUCAACCAUCUCAUAUGAAAGACUAUGUA
UACACUGAAAAAGAUAACGGUGGUGUGCAUACGAAUUCUGGCAUUCCAAAUAAAGCAGCUUAUAACGUAAU
UCAAGCAAUAGGGAAAUCUAAAUCAGAACAAAUUUACUACCGAGCAUUAACGGAAUACUUAACAAGUAAUU
CAAACUUCAAAGAUUGUAAAGAUGCAUUAUACCAAGCGGCUAAAGAUUUAUAUGACGAGCAAACAGCUGAA
CAAGUAUAUGAAGCAUGGAACGAAGUUGGCGUCGAGUAA
SEQ IN NO: 43
AUGAAAAAGAAAUUAGGUAUGUUACUUCUUGUACCAGCCGUAACUUUAUCAUUAGCCGCAUGUGGGAAUGA
UGAUGGAAAAGAUAAAGAUGGCAAGGUAACAAUUAAAACGACAGUUUAUCCAUUGCAAUCAUUUGCAGAGC
AAAUUGGUGGAAAACACGUGAAGGUAUCAUCAAUCUAUCCAGCAGGGACAGAUUUACAUAGCUAUGAACCA
ACACAAAAAGAUAUAUUAAGUGCAAGCAAGUCAGACUUGUUUAUGUAUACAGGGGAUAAUUUAGAUCCGGU
UGCUAAGAAAGUUGCAUCUACUAUUAAAGAUAAAGAUAAAAAACUGUCUUUAGAAGAUAAAUUAGAUAAAG
CAAAGCUUUUAACUGAUCAACACGAACAUGGUGAAGAGCAUGAACAUGAGGGACAUGAUCAUGGGAAAGAA
GAACAUCAUCAUCAUGGCGGAUAUGAUCCACACGUAUGGUUAGAUCCUAAAAUUAACCAAACUUUCGCUAA
AGAAAUUAAAGAUGAAUUAGUGAAGAAAGAUCCAAAACAUAAAGAUGACUAUGAGAAAAACUACAAAAAAU
UAAACGACGAUCUUAAGAAAAUUGAUAACGAUAUGAAGCAAGUUACUAAAGAUAAGCAAGGUAAUGCAGUA
UUCAUUUCACAUGAAUCAAUUGGAUACUUAGCUGAUCGUUAUGGUUUUGUUCAAAAAGGUAUUCAAAACAU
GAAUGCUGAAGAUCCAUCACAAAAAGAAUUGACUAAAAUUGUUAAAGAAAUUAGAGAUAGCAAUGCUAAAU
AUAUUCUUUACGAAGAUAAUGUUGCGAAUAAAGUGACUGAAACAAUUCGUAAAGAAACAGAUGCGAAGCCU
UUAAAAUUCUACAACAUGGAGUCUUUAAAUAAAGAACAACAGAAAAAAGAUAAUAUUACCUAUCAAUCAUU
AAUGAAAUCGAAUAUUGAAAAUAUCGGUAAAGCUUUAGACAGUGGUGUUAAAGUGAAAGACGACAAAGCUG
AAAGUAAACACGACAAAGCAAUUUCUGAUGGGUAUUUUAAAGAUGAGCAAGUUAAAGACCGUGAAUUAAGC
GAUUAUGCUGGUGAAUGGCAAUCUGUUUACCCUUACUUAAAAGACGGUACGCUUGAUGAAGUGAUGGAACA
UAAAGCUGAAAAUGAUCCGAAAAAUCUGCUAAAGAUUUAAAAGCUUAUUAUGACAAAGGAUAUAAAACUGA
UAUUACUAACAUUGAUAUAAAAGGAAAUGAAAUUACAUUUACUAAAGAUGGUACGAAACACACUGGUAAAU
AUGAAUACAAUGGUAAGAAAACAUUGAAAUAUCCUAAAGGUAACCGUGGCGUGAGAUUUAUGUUUAAAUUG
GUCGAUGGUAAUGAUAAAGACUUACCGAAAUUCAUCCAAUUUAGCGAUCACAACAUUGCACCUAAAAAGGC
AGAACACUUCCAUAUCUUUAUGGGUAAUGAUAAUGACGCGUUAUUAAAAGAAAUGGAUAACUGGCCAACAU
AUUAUCCUUCAAAAUUAAAUAAAGACCAAAUCAAAGAAGAAAUGUUAGCGCAUUAA
SEQ IN NO: 44
AUGGUGUUAUAUAUCAUUUUGGCAAUAAUUGUGAUUAUAUUGAUUGCUGUAGGUGUAUUAUUCUAUUUACG
UUCAAAUAAAAGACAAAUAAUAGAAAAAGCAAUCGAACGUAAAAAUGAAAUUGAAACGUUACCUUUUGAUC
AAAACCUUGCACAAUUAUCUAAGUUGAAUUUAAAAGGUGAAACAAAAACGAAAUACGAUGCAAUGAAAAAG
GACAACGUAGAAAGUACAAAUAAGUAUCUAGCUCCUGUGGAAGAAAAAAUCCAUAAUGCUGAGGCUUUAUU
AGAUAAAUUUAGUUUCAACGCAUCUCAAUGUGAAAUUGAUGAUGCAAAUGAGUUGAUGGAUAGUUACGAAC
AAAGCUAUCAGCAACAAUUAGAAGAUGUAAAUGAAAUUAUUGCGUUAUACAAAGAUAAUGAUGAAUUAUAU
GACAAAUGUAAGGUUGAUUAUCGUGAAAUGAAACGUGAUGUUUUAGCAAAUCGUCAUCAAUUUGGUGAGGC
AGCAAGUCUUCUUGAAACUGAAAUUGAAAAAUUCGAGCCAAGGUUAGAGCAAUAUGAAGUACUAAAAGCUG
AUGGUAAUUAUGUACAAGCGCACAACCAUAUAGCUGCCUUGAAUGAACAAAUGAAACAGCUAAGAUCUUAU
AUGGAAGAAAUACCAGAAUUAAUUAGAGAAACUCAAAAAGAAUUACCUGGUCAAUUCCAAGAUUUAAAAUA
UGGUUGCCGUGAUCUUAAAGUUGAAGGGUAUGAUCUGGAUCACGUAAAAGUAGACAGUACAUUACAAAGCU
UAAAAACAGAGCUUAGUUUCGUUGAACCAUUAAUUAGUCGCUUAGAAUUAGAAGAAGCUAAUGAUAAACUA
GCUAAUAUCAAUGAUAAGUUAGAUGACAUGUAUGAUUUAAUUGAACAUGAAGUUAAAGCUAAAAAUGAUGU
CGAAGAAACAAAAGAUAUCAUUACGGAUAACUUAUUCAAAGCAAAAGACAUGAAUUAUACAUUGCAAACAG
AAAUUGAAUAUGUACGUGAAAACUACUAUAUAAAUGAAUCUGAUGCUCAGAGUGUUCGUCAAUUUGAAAAU
GAAAUUCAAAGUUUAAUUUCUGUAUAUGAUGAUAUUUUAAAAGAAAUGUCUAAAUCUGCUGUACGAUAUAG
CGAGGUUCAGGAUAAUUUACAAUAUUUAGAAGAUCAUGUCACAGUUAUUAAUGACAAACAAGAAAAGCUAC
AAAAUCAUCUGAUUCAAUUGCGUGAAGAUGAAGCAGAAGCAGAAGACAAUCUGUUACGAGUACAAUCGAAG
AAAGAAGAAGUGUAUCGUCGAUUACUUGCUUCUAACUUAACAAGCGUUCCUGAAAGGUUUAUCAUCAUGAA
AAAUGAAAUUGAUCAUGAAGUUCGUGAUGUUAACGAACAAUUUAGUGAACGUCCAAUACACGUUAAACAGU
UAAAAGAUAAAGUGUCUAAAAUUGUGAUUCAAAUGAAUACAUUUGAAGAUGAAGCAAAUGAUGUUCUUGUU
AAUGCUGUUUAUGCAGAGAAAUUAAUUCAAUAUGGAAAUAGAUAUCGUAAGGACUAUAGCAAUGUUGAUAA
GAGCUUAAAUGAAGCUGAACGAUUAUUUAAAAAUAAUCGCUAUAAGCGUGCGAUUGAAAUUGCAGAGCAAG
CUCUUGAAAGUGUUGAGCCAGGUGUCACUAAACAUAUUGAAGAAGAAGUUAUUAAGCAAUAG
SEQ IN NO: 45
AUGCCUAAAAAUAAAAUUUUAAUUUAUUUGCUAUCAACUACGCUCGUAUUACCUACUUUAGUUUCACCUAC
CGCUUAUGCUGACACACCUCAAAAAGAUACUACAGCUAAGACAACAUCUCAUGAUUCCAAAAAAUCUACUG
AUGAUGAAACUUCUAAGGAUACUACAAGUAAAGAUAUUGAUAAAGCAGACAACAAUAAUACUAGUAACCAA
GACAAUAACGACAAAAAAGUUAAAACUAUAGACGACAGCACUUCAGACUCUAACAAUAUCAUUGAUUUUAU
UUAUAAGAAUUUACCACAAACCAAUAUAAACCAAUUGCUAACCAAAAAUAAAUACGAUGAUAAUUACUCAU
UAACAACUUUAAUCCAAAACUUAUUCAAUUUAAAUUCGGAUAUUUCUGAUUACGAACAACCUCGUAAUGGU
GAAAAGUCAACAAAUGAUUCGAAUAAAAACAGUGAUAAUAGCAUCAAAAAUGAUACGGAUACGCAAUCAUC
UAAACAAGAUAAAGCAGACAAUCAAAAAGCACCUAAAUCAAACAAUACAAAACCAAGUACAUCUAAUAAGC
AACCAAAUUCGCCAAAGCCAACACAACCAAAUCAAUCAAAUAGUCAACCAGCAAGUGACGAUAAAGUAAAU
CAAAAAUCUUCAUCGAAAGAUAAUCAAUCAAUGUCAGAUUCGGCUUUAGAUUCUAUUUUGGAUCAAUACAG
UGAAGAUGCAAAGAAAACACAAAAAGAUUACGCAUCUCAAUCUAAAAAAGACAAAAAUGAAAAAUCUAAUA
CAAAGAAUCCACAGUUACCAACACAAGAUGAAUUGAAACAUAAAUCUAAACCUGCUCAAUCAUUCAAUAAC
GAUGUUAAUCAAAAGGAUACACGUGCAACAUCACUAUUCGAAACAGAUCCUAGUAUAUCUAACAAUGAUGA
UAGUGGACAAUUUAACGUUGUUGACUCAAAAGAUACACGUCAAUUUGUCAAAUCAAUUGCUAAAGAUGCAC
ACCGCAUUGGUCAAGAUAACGAUAUUUAUGCGUCUGUCAUGAUUGCCCAAGCAAUCUUAGAAUCUGACUCA
GGUCGUAGUGCUUUAGCUAAGUCACCAAACCAUAAUUUAUUCGGUAUCAAAGGUGCUUUUGAAGGGAAUUC
UGUUCCUUUUAACACAUUAGAAGCUGAUGGUAAUCAAUUGUAUAGUAUUAAUGCUGGAUUCCGAAAAUAUC
CAAGCACGAAAGAAUCACUAAAAGAUUACUCUGACCUUAUUAAAAAUGGUAUUGAUGGCAAUCGAACAAUU
UAUAAACCAACAUGGAAAUCGGAAGCCGAUUCUUAUAAAGAUGCAACAUCACACUUAUCUAAAACAUAUGC
UACAGAUCCAAACUAUGCUAAGAAAUUAAACAGUAUUAUUAAACACUAUCAAUUAACUCAGUUUGACGAUG
AACGUAUGCCAGAUUUAGAUAAAUAUGAACGUUCUAUCAAGGAUUAUGAUGAUUCAUCAGAUGAAUUCAAA
CCUUUCCGCGAGGUAUCUGAUAAUAUGCCAUAUCCACAUGGCCAAUGUACUUGGUACGUAUAUAACCGUAU
GAAACAAUUUGGUACAUCUAUCUCAGGUGAUUUAGGUGAUGCACAUAAUUGGAAUAAUCGAGCUCAAUACC
GUGAUUAUCAAGUAAGUCAUACACCAAAACGUCAUGCUGCUGUUGUAUUUGAGGCUGGACAAUUUGGUGCA
GAUCAACAUUACGGUCAUGUAGCAUUUGUUGAAAAAGUUAACAGUGAUGGUUCUAUCGUUAUUUCAGAAUC
CAAUGUUAAAGGAUUAGGUAUCAUUUCUCAUAGAACUAUCAAUGCAGCUGCCGCUGAAGAAUUAUCAUAUA
UUACAGGUAAAUAA
SEQ IN NO: 46
AUGAUGAAAAGUCAAAAUAAGUAUAGUAUUCGUAAAUUUAGUGUAGGUGCAUCUUCCAUUUUAAUAGCUAC
AUUACUAUUUUUAAGUGGUGGACAAGCACAAGCAGCUGAGAAGCAAGUGAAUAUGGGAAAUUCACAGGAGG
AUACAGUUACAGCACAAUCUAUUGGGGAUCAACAAACUAGGGAAAAUGCUAAUUAUCAACGUGAAAACGGU
GUUGACGAACAGCAACAUACUGAAAAUUUAACUAAGAACUUGCAUAAUGAUAAAACAAUAUCAGAAGAAAA
UCAUCGUAAAACAGAUGAUUUGAAUAAAGAUCAACUAAAGGAUGAUAAAAAAUCAUCGCUUAAUAAUAAAA
AUAUUCAACGUGAUACAACAAAAAAUAACAAUGCUAAUCCUAGGGAUGUAAAUCAAGGGUUAGAACAGGCU
AUUAAUGAUGGCAAACAAAGUAAAGUGGCGUCACAGCAACAGUCAAAAGAGGCAGAUAAUAGUCAAGACUU
AAACGCUAAUAACAAUCUACCUUCACAAAGUCGAACAAAGGUAUCACCAUCAUUAAAUAAGUCAGAUCAAA
CAAGUCAACGAGAAAUUGUUAAUGAGACAGAAAUAGAGAAAGUACAACCGCAACAAAAGAAUCAAGCGAAU
GAUAAAAUUACUGACCACAAUUUUAACAAUGAACAAGAAGUGAAACCUCAAAAAGACGAAAAAACACUAUC
AGUUUCAGAUUUAAAAAACAAUCAAAAAUCACCAGUUGAACCAACAAAGGACAAUGACAAGAAAAAUGGAU
UAAAUUUAUUAAAAAGUAGUGCAGUAGCAACGUUACCAAACAAAGGGACAAAGGAACUUACUGCAAAAGCG
AAAGGUGAUCAAACGAAUAAAGUUGCCAAACAAGGGCAGUAUAAAAAUCAAGAUCCUAUAGUUUUAGUGCA
UGGUUUCAAUGGGUUUACAGAUGAUAUUAAUCCUUCAGUGUUAGCUCAUUAUUGGGGCGGUAAUAAAAUGA
ACAUUCGCCAAGAUUUAGAAGAAAAUGGUUACAAAGCUUAUGAAGCAAGUAUAAGUGCUUUUGGAAGUAAC
UAUGACCGCGCAGUUGAACUUUAUUAUUAUAUCAAAGGCGGUCGUGUAGAUUAUGGUGCAGCACAUGCAGC
AAAAUAUGGACAUGAACGUUAUGGAAAAACAUACGAAGGAAUUUACAAAGACUGGAAACCAGGACAGAAGG
UACACCUUGUUGGACAUAGUAUGGGUGGUCAAACGAUACGUCAACUAGAAGAAUUACUGCGUAAUGGUAGU
CGUGAAGAAAUAGAGUAUCAAAAGAAACAUAGUGGCGAAAUUUCUCCACUAUUCAAAGGUAAUAAUGACAA
UAUGAUUUCAUCAAUUACUACUUUAGGAACGCCACAUAAUGGAACGCAUGCUUCAGAUUUAGCUGGUAAUG
AAGCUUUAGUGAGACAAAUUGUAUUUGAUAUCGGUAAAAUGUUUGGUAAUAAAAAUUCAAGAGUAGACUUC
GGGUUGGCUCAAUGGGGUCUAAAACAGAAGCCAAAUGAAUCAUAUAUUGAUUAUGUCAAACGCGUUAAACA
AUCUAAUUUAUGGAAAUCAAAAGAUAAUGGAUUUUACGAUCUGACGCGUGAGGGUGCAACAGAUUUAAAUC
GUAAAACGUCGUUGAACCCUAACAUUGUGUAUAAAACAUACACUGGUGAAGCAACGCACAAAGCAUUAAAU
AGCGAUAGACAAAAAGCAGACUUAAAUAUGUUUUUCCCAUUUGUGAUUACUGGUAACUUAAUCGGUAAAGC
UACUGAAAAAGAAUGGCGAGAAAACGAUGGUUUAGUAUCCGUUAUUUCUUCUCAACAUCCAUUUAAUCAAG
CUUAUACAAAUGCGACGGAUAAAAUUCAAAAAGGCAUUUGGCAAGUAACGCCUACAAAACAUGAUUGGGAU
CAUGUUGAUUUUGUCGGACAAGAUAGUUCUGAUACAGUGCGCACAAGAGAAGAAUUACAAGAUUUUUGGCA
UCAUUUAGCAGACGAUUUAGUGAAAACUGAAAAGGUGACUGAUACUAAGCAAGCAUAA
SEQ IN NO: 47
AUGACAAAUAAAAUGAAGAAAUGGCAAAAAUUAUCCACCAUUACGUUAUUAAUGACCGGAGUGAUUGCUUU
AAAUAAUGGUGAAUUUAGAAAUGUUGAUAAACAUCAAAUCGCUGUGGCUGAUACGAAUGUUCAAACGCCAG
AUUAUGAAAAAUUGAAGAAGACGUGGCUCGACGUUAACUACGGUUAUGAUCAGUAUGAUGAGAAUAAUCAA
GAUAUGAAGAAGAAGUUUGAUGCUAAAGAAAAAGAAGCCAAGAAGUUACUUGAUGACAUGAAAACUGAUAC
GAAUAGAACAUAUUUGUGGUCAGGAGCUGAAAACCUUGAAACUAAUUCUUCUCACAUGACAAAAACCUAUC
GUAAUAUCGAGAAAAUCGCAGAAUCAAUGCAACAUAAGAAUACGGUAUUAAAAACAGUUGAAAACAAGUUG
AAAAUAAAAGAAGCCCUAGAUUGGAUGCACAAAAAUGUUUAUGGCAAGAAUCCUUCUCAAAAAGUCGAGGA
UUUAACUAAAAAUCGUAAGGGGCAAACUACACCCAAGAAUAACUCAUUGAAUUGGUGGGAUUAUGAAAUUG
GUACGCCAAGAGCAUUAACAAAUACACUACUUCUAAUGGAUGAUAUGCUCACUAAAGAUGAAAUGAAAAAU
UAUUCAAAACCUAUUAGUACAUAUGCACCAUCCAGUGACAAAAUUUUAUCUUCUGUUGGUGAAUCAGAAGA
UGCUAAAGGUGGAAAUUUAGUGGACAUUUCUAAAGUAAAACUUUUAGAAAGUGUUAUUGAAGAAGAUGUAG
AUAUGUUGAAAAAGUCUAUAGAUUCUUUUAAUAAAGUGUUCACUUAUGUUCAAGAUUCUGCCACUGGUAAA
GGUCGCAAUGGAUUCUAUAAAGAUGGCUCUUACAUUGAUCAUCAAGAUGUCCCUUACACUGGUGCUUAUGG
UGUUGUACUAUUAGAGGGUAUUUCUCAAAUGAUGCCGAUGAUAAAAGAAUCUCCUUUUAAAACUACACAAG
AUAAUGCUACAUUAAGCAAUUGGAUUGACGAAGGGUUUAUGCCAUUAAUCUAUAAAGGUGAAAUGAUGGAU
UUAUCACGAGGUAGAGCUAUCAGUCGUGAAAAUGAAACGAGUCAUACAGCGUCAGCGACUGUAAUGAAAUC
AUUGUUGAGAUUGAAUGAUACCAUGGAUGAUUCAACAAAAACUAGAUAUAAGCAAAUCGUUAAAACUUCUG
UUAAUUCUGAUUCAAGUUACAACCAAAAUAAUUAUUUAAAUUCAUAUUCAGACAUAGCUAAAAUGAAAAAG
UUAAUGAAUGAUAGUACUAUUUCUAAAAACGAUUUAACACAGCAACUUAAAAUAUAUAAUGACAUGGAUCG
UGUCACCUAUCACAAUAAAGACCUGGACUUUGCAUUUGGUUUAAGUAUGACAUCGAAAAACAUCGCACGAU
ACGAAAAUAUCAACGGAGAGAACUUAAAAGGUUGGCACACCGGUGCAGGCAUGUCUUAUUUAUAUAACAGC
GAUGUCAAACACUAUCGCGAUAACUUCUGGGCAACAGCCGAUAUGACUUGUCUUCCAGGCACUACUACUUU
AAAUGAUAUGCCAUCUACUAAUACUAAGAAUGAUAAAUCUUUUGUUGGCGGGACAAAAUUAAAUAAUAAAU
ACGCAAGCAUCGGUAUGGAUUUUGAAAAUCAGGACAAAACUUUAACUGCCAAAAAAUCAUAUUUCAUAUUA
AACGAUAAAAUUGUCUUCUUAGGAACUGGCAUUAAAAGUACUGAUUCAUCAAAGAAUCCAGUUACAAGUGU
UGAAAAUCGCAAAGCAAAUGGGUAUAAAUUAUUUAAAGAUGAUAUUGAAAUUACCACUUCAGAUGUUAAUG
CUCAGGAAACCCAUUCAGUCUUUUUAGAGUCCAACGAUACUAAAAAGAACAUUGGUUAUCAUUUCUUAGAC
AAGCCAAAAAUAACUGUAAAAAAAGAAAGUCAUACUGGUAAGUGGAGUGAAAUUAAUAAAAGUCAAAAAAA
AGAUGACAAAAAAGAUGAGUAUUAUGAAGUAACUCAAACACAUAAUACAUCUGACAGUAAAUAUGCAUAUG
UUUUGUAUCCUGGUUUAUCAAAAAGUGAUUUUAAAUCGAAGAAUAAUAAUGUAAGUAUUGUUAAACAAGAU
GAAGAUUUUCAUGUGAUAAAAGAUAAUGAUGGCGUAUUUGCUGGGGUUAAUUAUAGUGAUAAUACUAAAUC
UUUUGAUAUAAACGGAAUUACUGUUGAAUUAAAAGAAAAAGGCAUGUUUGUAAUUAAAAAGAAAGAUGAUA
AAGCAUAUAAAUGUAGCUUCUAUAAUCCUGAAACUACAAAUACCGCUUCAAAUAUAGAAUCAAAAAUUUUU
AUUAAAGGUUACACCAUAACUAAUAAAAGUGUCAUAAACUCUAAUGAUGCUGGUGUAAACUUUGAAUUAAC
UAAAUAA
SEQ IN NO: 48
AUGACAUAUAGAAUGAAGAAAUGGCAAAAAUUGUCCACCAUUACGUUAUUAAUGGCUGGUGUGAUUACUUU
GAAUGGUGGUGAAUUCAGAAGUAUUGAUAAACAUCAAAUCGCUGUGGCUGAUACGAAUGUUCAAACGACAG
AUUAUGAAAAGUUGAGGAACAUAUGGCUGGACGUUAACUAUGGUUAUGAUAAGUAUGAUGAGAAUAAUCCA
GAUAUGAAGAAGAAGUUUGAGGCUACGGAGAAUGAGGCAGAGAAAUUACUCAAGGAAAUGAAAACUGAAAG
UGAUAGGAAAUACUUGUGGGAAAGCUCAAAAGAUUUAGAUACGAAGUCUGCGGAUAUGACUCGUACCUAUC
GUAAUAUUGAGAAAAUCUCAGAAGCGAUGAAACAUAAAAAUACUAAAUUAAAAACAGAUGAAAACAAGACA
AAAGUAAAAGAUGCACUUGAGUGGCUGCAUAAAAAUGCAUAUGGAAAAGAACCAGAUAAAAAAGUUGCUGA
UUUGACCUCAAACUUUAAAAAUAAAACUUCUAGAAAUACCAACUUAAAUUGGUGGGAUUAUGAAAUUGGAA
CACCUAGAGCAUUAACAAAUACGCUUAUACUCUUACAAGAAGAUUUCACUGAUGAAGAAAAGAAAAAAUAU
ACAGCUCCUAUUAAAACUUUCGCCCCAGAUAGUGACAAAAUAUUAUCUUCUGUAGGAAAAUCUGAACCUGC
UAAAGGCGGAAAUUUAGUAGACAUUUCUAAAGUAAAACUUUUAGAAAGUAUUAUCGAAGAAGACAAAGAUA
UGAUGAAAAAGUCUAUAGAUUCAUUUAAUACAGUCUUCACUUACGCGCAAAAUUCUGCCACUGGAAAAGAA
CGUAAUGGAUUCUAUAAAGAUGGCUCUUACAUUGAUCAUCAAGACGUCCCAUACACUGGUGCUUAUGGCGU
UGUACUAUUAGAGGGUAUUUCUCAAAUGAUGCCGAUGAUAAAAGAAACACCUUUUAAUGAUAGUAACCAAA
AUGAUACAACCUUAAAAUCAUGGAUUGACGACGGAUUUAUGCCACUCAUUUAUAAAGGUGAAAUGAUGGAU
UUAUCAAGAGGUAGAGCUAUCAGUCGUGAAAAUGAAACGAGUCACUCAGCAUCUGCAACAGUAAUGAAAUC
AUUGUUGAGAUUGAGUGAUACCAUGGAUAAGUCUACAAAAGCUAAAUAUAAAAAGAUUGUCAAGACUUCAG
UAGAGUCAGAUUCAAGUUAUAAACAAACCGAUUAUUUAAGCUCUUAUUCGGAUAUAAGCAAAAUGAAGUCU
UUAAUGGAAGACAGCACUAUUUCUACUAACGGUUUAACACAACAACUUAAAAUAUAUAAUGACAUGGAUCG
UGUCACCUAUCACAAUAAAGGCUUAGACUUUGCAUUUGGUUUAAGUAUGACGUCGAAAAACGUCGCACGUU
ACGAAAGUAUCAACGGAGAGAACUUAAAAGGUUGGCACACUGGUGCUGGAAUGUCUUAUUUAUACAAUAGC
GAUGUGAAACACUACCGUGAUAACUUCUGGGCGACAGCUGAUAUGAAACGUUUAGCAGGUACUACAACUUU
AGAUAAUGAAGAACCUAAAAGUACGGAUGUUAAAAAGUCUAGUAAAACUUUUGUAGGAGGAACAAAAUUCG
AUGACCAACAUGCUAGUAUCGGAAUGGAUUUUGAAAAUCAGGACAAAACUUUAACUGCCAAAAAAUCAUAU
UUCAUAUUAAACGAUAAAAUUGUCUUCUUAGGAACUGGCAUUAAAAGUACUGAUUCAUCAAAGAAUCCAGU
UACAACGAUUGAAAAUCGCAAAGCGAAUGAUUAUAAAUUAUAUAAAGAUGAUACGCAAACAACCAAUUCCG
AUAAUCAGGAAACCAAUUCCCUCUUUUUAGAGUCAACGAAUAGCACUCAAAACAAUAUAGGUUAUCAUUUU
UUAAACGAAUCGAAAAUAACUGUAAAAAAAGAAAGUCAUACUGGUAAGUGGAGUGAUAUAAAUAAAAGCCA
AAAGGAUAUACAAAAAACUGAUGAGUAUUAUGAAGUAACUCAAAAGCAUUCUAAUACAGAUAGUAAAUAUG
CAUAUGUGUUGUAUCCAGGCUUAUCUAAAGAUGUCUUUAAAUCCAAAGCAAGCAAAGUAACUGUCGUUAAG
CAAGAAGAUGACUUCCACGUUGUGAAAGAUAAUGAAUCGGUUUGGGCUGGUAUCAAUUAUAGUGAUAGCGC
UAAAACUUUUGAAAUUAAUAACACUAAAGUCGAAGUUAAAGCCAAAGGAAUGUUUAUUCUUACAAAGAAAG
AUGAUAACACUUAUGAAUGUAGCUUCUAUAAUCCCGAAUCUACAAAUUCCGUUUCAGAUAUUGAAUCUAAA
AUUUCAAUGACUGGAUACUCUAUUAUAAACAAAAAUACGUCGACUUCUAAUGAAUCCGGCGUACGCUUUGA
AUUAACUAAAUAA
SEQ ID NO: 50
ATTGATTCAAAAAATAAACCAGCTAATTCTGATATTAAATTTGAGGTGACTCAAAAGAGTGATGCGGTCAA
AGCATTAAAAGAATTGCCTAAATCCGAAAATGTAAAAAATATTTATCAAGATTACGCTGTTACTGATGTAA
AAACTGATAAAAAAGGATTTACGCATTATACATTGCAACCGAGTGTTGATGGTGTTCATGCACCTGACAAA
GAAGTGAAAGTACACGCAGACAAATCAGGAAAAGTCGTTTTAATCAATGGGGATACTGATGCGAAGAAAGT
AAAGCCAACGAATAAAGTGACATTAAGTAAAGATGACGCAGCCGACAAAGCATTTAAAGCAGTTAAGATTG
ATAAGAATAAAGCGAAAAATCTTAAAGATAAAGTCATTAAAGAAAACAAAGTTGAAATCGATGGTGACAGT
AATAAATACGTTTATAATGTTGAGTTAATTACAGTGACACCAGAAATTTCACATTGGAAAGTTAAAATTGA
TGCTCAAACTGGCGAAATTTTAGAAAAAATGAACTTAGTTAAAGAAGCTGCAGAAACTGGTAAAGGAAAAG
GTGTACTTGGCGATACAAAAGATATCAATATCAATAGTATTGACGGTGGATTTAGCCTAGAAGATTTAACG
CATCAAGGTAAATTATCAGCATTTAGCTTTAATGATCAAACAGGTCAAGCAACATTGATTACTAATGAAGA
TGAAAACTTCGTAAAAGATGAGCAACGTGCTGGCGTAGATGCAAATTATTACGCTAAACAAACATATGATT
ATTACAAAGACACATTTGGTCGTGAATCATATGACAACCAAGGTAGTCCAATTGTTTCATTAACGCATGTT
AATAACTACGGTGGTCAAGATAACAGAAATAATGCCGCATGGATCGGTGACAAAATGATCTATGGTGATGG
TGATGGTCGCACATTCACAAGTTTATCGGGTGCAAATGACGTAGTAGCACACGAATTAACACACGGTGTGA
CACAAGAGACAGCGAACTTAGAATATAAGGACCAGTCAGGCGCTCTAAATGAAAGCTTTTCAGATGTTTTT
GGATACTTTGTAGATGACGAGGATTTCTTAATGGGTGAAGATGTCTACACACCTGGAAAAGAGGGAGACGC
TTTACGCAGCATGTCAAACCCAGAACAATTTGGTCAACCAGCTCATATGAAAGACTATGTATTCACTGAAA
AAGATAATGGTGGCGTACATACGAATTCTTAA
SEQ ID NO: 51
AUUGAUUCAAAAAAUAAACCAGCUAAUUCUGAUAUUAAAUUUGAGGUGACUCAAAAGAGUGAUGCGGUCAA
AGCAUUAAAAGAAUUGCCUAAAUCCGAAAAUGUAAAAAAUAUUUAUCAAGAUUACGCUGUUACUGAUGUAA
AAACUGAUAAAAAAGGAUUUACGCAUUAUACAUUGCAACCGAGUGUUGAUGGUGUUCAUGCACCUGACAAA
GAAGUGAAAGUACACGCAGACAAAUCAGGAAAAGUCGUUUUAAUCAAUGGGGAUACUGAUGCGAAGAAAGU
AAAGCCAACGAAUAAAGUGACAUUAAGUAAAGAUGACGCAGCCGACAAAGCAUUUAAAGCAGUUAAGAUUG
AUAAGAAUAAAGCGAAAAAUCUUAAAGAUAAAGUCAUUAAAGAAAACAAAGUUGAAAUCGAUGGUGACAGU
AAUAAAUACGUUUAUAAUGUUGAGUUAAUUACAGUGACACCAGAAAUUUCACAUUGGAAAGUUAAAAUUGA
UGCUCAAACUGGCGAAAUUUUAGAAAAAAUGAACUUAGUUAAAGAAGCUGCAGAAACUGGUAAAGGAAAAG
GUGUACUUGGCGAUACAAAAGAUAUCAAUAUCAAUAGUAUUGACGGUGGAUUUAGCCUAGAAGAUUUAACG
CAUCAAGGUAAAUUAUCAGCAUUUAGCUUUAAUGAUCAAACAGGUCAAGCAACAUUGAUUACUAAUGAAGA
UGAAAACUUCGUAAAAGAUGAGCAACGUGCUGGCGUAGAUGCAAAUUAUUACGCUAAACAAACAUAUGAUU
AUUACAAAGACACAUUUGGUCGUGAAUCAUAUGACAACCAAGGUAGUCCAAUUGUUUCAUUAACGCAUGUU
AAUAACUACGGUGGUCAAGAUAACAGAAAUAAUGCCGCAUGGAUCGGUGACAAAAUGAUCUAUGGUGAUGG
UGAUGGUCGCACAUUCACAAGUUUAUCGGGUGCAAAUGACGUAGUAGCACACGAAUUAACACACGGUGUGA
CACAAGAGACAGCGAACUUAGAAUAUAAGGACCAGUCAGGCGCUCUAAAUGAAAGCUUUUCAGAUGUUUUU
GGAUACUUUGUAGAUGACGAGGAUUUCUUAAUGGGUGAAGAUGUCUACACACCUGGAAAAGAGGGAGACGC
UUUACGCAGCAUGUCAAACCCAGAACAAUUUGGUCAACCAGCUCAUAUGAAAGACUAUGUAUUCACUGAAA
AAGAUAAUGGUGGCGUACAUACGAAUUCUUAA

The polypeptides of the present invention are also designated as follows herein:

| | |
|---|---|
| SEQ ID NO: 1: | SAR1837 |
| SEQ ID NO: 2: | SAR1608 |
| SEQ ID NO: 3: | SAR2457 |
| SEQ ID NO: 4: | SAR0839 |
| SEQ ID NO: 5: | SAR1402 |
| SEQ ID NO: 6: | SAR1022 |
| SEQ ID NO: 7: | SAR1995 |
| SEQ ID NO: 8: | SAR0436 |
| SEQ ID NO: 49: | USA300HOU_2637_28_439 |
| SEQ ID NO: 9: | SAR0277 |
| SEQ ID NO: 10: | SAR2716 |
| SEQ ID NO: 11: | SAR2496 |
| SEQ ID NO: 12: | SAR1795 |
| SEQ ID NO: 13: | SAR2723 |
| SEQ ID NO: 14: | SAR2753 |
| SEQ ID NO: 15: | SAR1892 |
| SEQ ID NO: 16: | SAR2292 |

1. A pharmaceutical composition comprising
a pharmaceutically acceptable carrier, vehicle or diluent, and an immunological adjuvant, and
a polypeptide comprising
a) SEQ NO: 13, or
b) an amino acid sequence consisting of at least or exactly or at most 35 contiguous amino acid residues from SEQ ID NO: 13, or
c) an amino acid sequence having a sequence identity of at least 60% with the amino acid sequence of a) or b).

2. The pharmaceutical composition according to claim 1, wherein the at least or exactly or at most 35 contiguous amino acids of option b are selected from at least or exactly or at most 36, at least or exactly or at most 37, at least or exactly or at most 38, at least or exactly or at most 39, at least or exactly or at most 40, at least or exactly or at most 41, at least or exactly or at most 42, at least or exactly or at most 43, at least or exactly or at most 44, at least or exactly or at most 45, at least or exactly or at most 46, at least or exactly or at most 47, at least or exactly or at most 48, at least or exactly or at most 49, at least or exactly or at most 50, at least or exactly or at most 51, at least or exactly or at most 52, at least or exactly or at most 53, at least or exactly or at most 54, at least or exactly or at most 55, at least or exactly or at most 56, at least or exactly or at most 57, at least or exactly or at most 58, at least or exactly or at most 59, at least or exactly or at most 60, at least or exactly or at most 61, at least or exactly or at most 62, at least or exactly or at most 63, at least or exactly or at most 64, at least or exactly or at most 65, at least or exactly or at most 66, at least or exactly or at most 67, at least or exactly or at most 68, at least or exactly or at most 69, at least or exactly or at most 70, at least or exactly or at most 71, at least or exactly or at most 72, at least or exactly or at most 73, at least or exactly or at most 74, at least or exactly or at most 75, at least or exactly or at most 76, at least or exactly or at most 77, at least or exactly or at most 78, at least or exactly or at most 79, at least or exactly or at most 80, at least or exactly or at most 81, at least or exactly or at most 82, at least or exactly or at most 83, at least or exactly or at most 84, at least or exactly or at most 85, at least or exactly or at most 86, at least or exactly or at most 87, at least or exactly or at most 88, at least or exactly or at most 89, at least or exactly or at most 90, at least or exactly or at most 91, at least or exactly or at most 92, at least or exactly or at most 93, at least or exactly or at most 94, at least or exactly or at most 95, at least or exactly or at most 96, at least or exactly or at most 97, at least or exactly or at most 98, at least or exactly or at most 99, at least or exactly or at most 100, at least or exactly or at most 101, at least or exactly or at most 102, at least or exactly or at most 103, at least or exactly or at most 104, at least or exactly or at most 105, at least or exactly or at most 106, at least or exactly or at most 107, at least or exactly or at most 108, at least or exactly or at most 109, at least or exactly or at most 110, at least or exactly or at most 111, at least or exactly or at most 112, at least or exactly or at most 113, at least or exactly or at most 114, at least or exactly or at most 115, at least or exactly or at most 116, at least or exactly or at most 117, at least or exactly or at most 118, at least or exactly or at most 119, at least or exactly or at most 120, at least or exactly or at most 121, at least or exactly or at most 122, at least or exactly or at most 123, at least or exactly or at most 124, at least or exactly or at most 125, at least or exactly or at most 126, at least or exactly or at most 127, at least or exactly or at most 128, at least or exactly or at most 129, at least or exactly or at most 130, at least or exactly or at most 131, at least or exactly or at most 132, at least or exactly or at most 133, at least or exactly or at most 134, at least or exactly or at most 135, at least or exactly or at most 136, at least or exactly or at most 137, at least or exactly or at most 138, at least or exactly or at most 139 contiguous amino acid residues, at least or exactly or at most 140, at least or exactly or at most 141, at least or exactly or at most 142, at least or exactly or at most 143, at least or exactly or at most 144, at least or exactly or at most 145, at least or exactly or at most 146, at least or exactly or at most 147, at least or exactly or at most 148, at least or exactly or at most 149, at least or exactly or at most 150, at least or exactly or at most 151, at least or exactly or at most 152, at least or exactly or at most 153, at least or exactly or at most 154, at least or exactly or at most 155, at least or exactly or at most 156, at least or exactly or at most 157, at least or exactly or at most 158, at least or exactly or at most 159, at least or exactly or at most 160, at least or exactly or at most 161, at least or exactly or at most 162, at least or exactly or at most 163, at least or exactly or at most 164, at least or exactly or at most 165, at least or exactly or at most 166, at least or exactly or at most 167, at least or exactly or at most 168, at least or exactly or at most 169, at least or exactly or at most 170, at least or exactly or at most 171, at least or exactly or at most 172, at least or exactly or at most 173, at least or exactly or at most 174, at least or exactly or at most 175, at least or exactly or at most 176, at least or exactly or at most 177, at least or exactly or at most 178, at least or exactly or at most 179, at least or exactly or at most 180, at least or exactly or at most 181, at least or exactly or at most 182, at least or exactly or at most 183, at least or exactly or at most 184, at least or exactly or at most 185, at least or exactly or at most 186, at least or exactly or at most 187, at least or exactly or at most 188, at least or exactly or at most 189, at least or exactly or at most 190, at least or exactly or at most 191, at least or exactly or at most 192, at least or exactly or at most 193, at least. or exactly or at most 194, at least or exactly or at most 195, at least or exactly or at most 196, at least or exactly or at most 197, at least or exactly or at most 198, at least or exactly or at most 199, at least or exactly or at most 200, at least or exactly or at most 201, at least or exactly or at most 202, at least or exactly or at most 203, at least or exactly or at most 204, at least or exactly or at most 205, at least or exactly or at most 206, at least or exactly or at most 207, at least or exactly or at most 208, at least or exactly or at most 209, at least or exactly or at most 210, at least or exactly or at most 211, at least or exactly or at most 212, at least or exactly or at most 213, at least or exactly or at most 214, at least or exactly or at most 215, at least or exactly or at most 216, at least or exactly or at most 217, at least or exactly or at most 218, at least or exactly or at most 219, at least or exactly or at most 220, at least or exactly or at most 221, at least or exactly or at most 222, at least or exactly or at most 223, at least or exactly or at most 224, at least or exactly or at most 225, at least or exactly or at most 226, at least or exactly or at most 227, at least or exactly or at most 228, at least or exactly or at most 229, at least or exactly or at most 230, at least or exactly or at most 231, at least or exactly or at most 232, at least or exactly or at most 233, at least or exactly or at most 234, at least or exactly or at most 235, at least or exactly or at most 236, at least or exactly or at most 237, at least or exactly or at most 238, at least or exactly or at most 239, at least or exactly or at most 240, at least or exactly or at most 241, at least or exactly or at most 242, at least or exactly or at most 243, at least or exactly or at most 244, at least or exactly or at most 245, at least or exactly or at most 246, at least or exactly or at most 247, at least or exactly or at most 248, at least or exactly or at most 249, at least or exactly or at most 250, at least or exactly or at most 251, at least or exactly or at most 252, at least or exactly or at most 253, at least or exactly or at most 254, at least or exactly or at most 255, at least or exactly or at most 256, at least or exactly or at most 257, at least or exactly or at most 258, at least or exactly or at most 259, at least or exactly or at most 260, at least or exactly or at most 261, at least or exactly or at most 262, at least or exactly or at most 263, at least or exactly or at most 264, at least or exactly or at most 265, at least or exactly or at most 266, at least or exactly or at most 267, at least or exactly or at most 268, at least or exactly or at most 269, at least or exactly or at most 270, at least or exactly or at most 271, at least or exactly or at most 272, at least or exactly or at most 273, at least or exactly or at most 274, at least or exactly or at most 275, at least or exactly or at most 276, at least or exactly or at most 277, at least or exactly or at most 278, at least or exactly or at most 279, at least or exactly or at most 280, at least or exactly or at most 281, at least or exactly or at most 282, at least or exactly or at most 283, at least or exactly or at most 284, at least or exactly or at most 285, at least or exactly or at most 286, at least or exactly or at most 287, at least or exactly or at most 288, at least or exactly or at most 289, at least or exactly or at most 290, at least or exactly or at most 291, at least or exactly or at most 292, at least or exactly or at most 293, at least or exactly or at most 294, at least or exactly or at most 295, at least or exactly or at most 296, at least or exactly or at most 297, at least or exactly or at most 298, at least or exactly or at most 299, at least or exactly or at most 300, at least or exactly or at most 301, at least or exactly or at most 302, at least or exactly or at most 303, at least or exactly or at most 304, at least or exactly or at most 305, at least or exactly or at most 306, at least or exactly or at most 307, at least or exactly or at most 308, at least or exactly or at most 309, at least or exactly or at most 310, at least or exactly or at most 311, at least or exactly or at most 312, at least or exactly or at most 313, at least or exactly or at most 314, at least or exactly or at most 315, at least or exactly or at most 316, at least or exactly or at most 317, at least or exactly or at most 318, at least or exactly or at most 319, at least or exactly or at most 320, at least or exactly or at most 321, at least or exactly or at most 322, at least or exactly or at most 323, at least or exactly or at most 324, at least or exactly or at most 325, at least or exactly or at most 326, at least or exactly or at most 327, at least or exactly or at most 328, at least or exactly or at most 329, at least or exactly or at most 330, at least or exactly or at most 331, at least or exactly or at most 332, at least or exactly or at most 333, at least or exactly or at most 334, at least or exactly or at most 335, at least or exactly or at most 336, at least or exactly or at most 337, at least or exactly or at most 338, at least or exactly or at most 339, at least or exactly or at most 340, at least or exactly or at most 341, at least or exactly or at most 342, at least or exactly or at most 343, at least or exactly or at most 344, at least or exactly or at most 345, at least or exactly or at most 346, at least or exactly or at most 347, at least or exactly or at most 348, at least or exactly or at most 349, at least or exactly or at most 350, at least or exactly or at most 351, at least or exactly or at most 352, at least or exactly or at most 353, at least or exactly or at most 354, at least or exactly or at most 355, at least or exactly or at most 356, at least or exactly or at most 357, at least or exactly or at most 358, at least or exactly or at most 359, at least or exactly or at most 360, at least or exactly or at most 361, at least or exactly or at most 362, at least or exactly or at most 363, at least or exactly or at most 364, at least or exactly or at most 365, at least or exactly or at most 366, at least or exactly or at most 367, at least or exactly or at most 368, at least or exactly or at most 369, at least or exactly or at most 370, at least or exactly or at most 371, at least or exactly or at most 372, at least or exactly or at most 373, at least or exactly or at most 374, at least or exactly or at most 375, at least or exactly or at most 376, at least or exactly or at most 377, at least or exactly or at most 378, at least or exactly or at most 379, at least or exactly or at most 380, at least or exactly or at most 381, at least or exactly or at most 382, at least or exactly or at most 383, at least or exactly or at most 384, at least or exactly or at most 385, at least or exactly or at most 386, at least or exactly or at most 387, at least or exactly or at most 388, at least or exactly or at most 389, at least or exactly or at most 390, at least or exactly or at most 391, at least or exactly or at most 392, at least or exactly or at most 393, at least or exactly or at most 394, at least or exactly or at most 395, at least or exactly or at most 396, at least or exactly or at most 397, at least or exactly or at most 398, at least or exactly or at most 399, at least or exactly or at most 400, at least or exactly or at most 401, at least or exactly or at most 402, at least or exactly or at most 403, at least or exactly or at most 404, at least or exactly or at most 405, at least or exactly or at most 406, at least or exactly or at most 407, at least or exactly or at most 408, at least or exactly or at most 409, at least or exactly or at most 410, at least or exactly or at most 411, at least or exactly or at most 412, at least or exactly or at most 413, at least or exactly or at most 414, at least or exactly or at most 415, at least or exactly or at most 416, at least or exactly or at most 417, at least or exactly or at most 418, at least or exactly or at most 419, at least or exactly or at most 420, at least or exactly or at most 421, at least or exactly or at most 422, at least or exactly or at most 423, at least or exactly or at most 424, at least or exactly or at most 425, at least or exactly or at most 426, at least or exactly or at most 427, at least or exactly or at most 428, at least or exactly or at most 429, at least or exactly or at most 430, at least or exactly or at most 431, at least or exactly or at most 432, at least or exactly or at most 433, at least or exactly or at most 434, at least or exactly or at most 435, at least or exactly or at most 436, at least or exactly or at most 437, at least or exactly or at most 438, at least or exactly or at most 439, at least or exactly or at most 440, at least or exactly or at most 441, at least or exactly or at most 442, at least or exactly or at most 443, at least or exactly or at most 444, at least or exactly or at most 445, at least or exactly or at most 446, at least or exactly or at most 117, at least or exactly or at most 448, at least or exactly or at most 449, at least or exactly or at most 450, at least or exactly or at most 451, at least or exactly or at most 452, at least or exactly or at most 453, at least or exactly or at most 454, at least or exactly or at most 455, at least or exactly or at most 456, at least or exactly or at most 457, at least or exactly or at most 458, at least or exactly or at most 459, at least or exactly or at most 460, at least or exactly or at most 461, at least or exactly or at most 462, at least or exactly or at most 463, at least or exactly or at most 464, at least or exactly or at most 465, at least or exactly or at most 466, at least or exactly or at most 467, at least or exactly or at most 468, at least or exactly or at most 469, at least or exactly or at most 470, at least or exactly or at most 471, at least or exactly or at most 472, at least or exactly or at most 473, at least or exactly or at most 474, at least or exactly or at most 475, at least or exactly or at most 476, at least or exactly or at most 477, at least or exactly or at most 478, at least or exactly or at most 479, at least or exactly or at most 480, at least or exactly or at most 481, at least or exactly or at roost 482, at least or exactly or at most 483, at least or exactly or at most 484, at least or exactly or at most 485, at least or exactly or at most 486, at least or exactly or at most 487, at least or exactly or at most 488, at least or exactly or at most 489, at least or exactly or at most 490, at least or exactly or at most 491, at least or exactly or at most 492, at least or exactly or at most 493, at least or exactly or at most 494, at least or exactly or at most 495, at least or exactly or at most 496, at least or exactly or at most 497, at least or exactly or at most 498, at least or exactly or at most 499, at least or exactly or at most 500, at least or exactly or at most 501, at least or exactly or at most 502, at least or exactly or at most 503, at least or exactly or at most 504, at least or exactly or at most 505, at least or exactly or at most 506, at least or exactly or at most 507 contiguous amino acid residues or at least or exactly or at most 508, at least or exactly or at most 509, at least or exactly or at most 510, at least or exactly or at most 511, at least or exactly or at most 512, at least or exactly or at most 513, at least or exactly or at most 514, at least or exactly or at most 515, at least or exactly or at most 516, at least or exactly or at most 517, at least or exactly or at most 518, at least or exactly or at most 519, at least or exactly or at most 520, at least or exactly or at most 521, at least or exactly or at most 522, at least or exactly or at most 523, at least or exactly or at most 524, at least or exactly or at most. 525, at least or exactly or at most 526, at least or exactly or at most 527, at least or exactly or at most 528, at least or exactly or at most 529, at least or exactly or at most 530, at least or exactly or at most 531, at least or exactly or at most 532, at least or exactly or at most 533, at least or exactly or at most 534, at least or exactly or at most 535, at least or exactly or at most 536, at least or exactly or at most 537, at least or exactly or at most 538, at least or exactly or at most 539, at least or exactly or at most 540, at least or exactly or at most 541, at least or exactly or at most 542, at least or exactly or at most 543, at least or exactly or at most 544, at least or exactly or at most 545, at least or exactly or at most 546, at least or exactly or at most 547, at least or exactly or at most 548, at least or exactly or at most 549, at least or exactly or at most 550, at least or exactly or at most 551, at least or exactly or at most 552, at least or exactly or at most 553, at least or exactly or at most 554, at least or exactly or at most 555, at least or exactly or at most 556, at least or exactly or at most 557, at least or exactly or at most 558, at least or exactly or at most 559, at least or exactly or at most 560, at least or exactly or at most 561, at least or exactly or at most 562, at least or exactly or at most 563, at least or exactly or at most 564, at least or exactly or at most 565, at least or exactly or at most 566, at least or exactly or at most 567, at least or exactly or at most 568, at least or exactly or at most 569, at least or exactly or at most 570, at least or exactly or at most 571, at least or exactly or at most 572, at least or exactly or at most 573, at least or exactly or at most 574, at least or exactly or at most 575, at least or exactly or at most 576, at least or exactly or at most 577, at least or exactly or at most 578, at least or exactly or at most 579, at least or exactly or at most 580, at least or exactly or at most 581, at least or exactly or at most 582, at least or exactly or at most 583, at least or exactly or at most 584, at least or exactly or at most 585, at least or exactly or at most 586, at least or exactly or at most 587, at least or exactly or at most 588, at least or exactly or at most 589, at least or exactly or at most 590, at least or exactly or at most 591, at least or exactly or at most 592, at least or exactly or at most 593, at least or exactly or at most 594, at least or exactly or at most 595, at least or exactly or at most 596, at least or exactly or at most 597, at least or exactly or at most 598, at least or exactly or at most 599, at least or exactly or at most 600, at least or exactly or at most 601, at least or exactly or at most 602, at least or exactly or at most 603, at least or exactly or at most 604, at least or exactly or at most 605. at least or exactly or at most 606, at least or exactly or at most 607, at least or exactly or at most 608, at least or exactly or at most 609, at least or exactly or at most 610, at least or exactly or at most 611, at least or exactly or at most 612, at least or exactly or at most 613, at least or exactly or at most 614, at least or exactly or at most 615, at least or exactly or at most 616, at least or exactly or at most 617, and exactly or at most 618 contiguous amino acid residues.

3. The pharmaceutical composition to claim 1, wherein the sequence identity is selected from at least 65%, at least 70%, at least 75%, at least 80%, at least 85%, at least 90%, at least 91%, at least 92%, at least 93%, at least 94%, at least 95%, at least 96%, at least 97%, at least 98%, and at least 99%.

4. The pharmaceutical composition according to claim 1, wherein the at least or exactly or at most 5 contiguous amino acid residues has an N-terminal amino acid residue corresponding to any one of amino acid residues 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 42, 43, 44, 45, 46, 47, 48, 49, 50, 51, 52, 53, 54, 55, 56, 57, 58, 59, 60, 61, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 110, 111, 112, 113, 114, 115, 116, 117, 118, 119, 120, 121, 122, 123, 124, 125, 126, 127, 128, 129, 130, 131, 132, 133, 134. 135, 136, 137, 138, 139, 140, 141, 142, 143, 144, 145, 146, 147, 148, 149, 150, 151, 152, 153, 154, 155, 156, 157, 158, 159, 160, 171, 172, 173, 174, 175, 176, 177, 178, 179, 180, 181, 182, 183, 184, 185. 186, 187. 188, 189, 190, 191, 192, 193, 194, 195, 196, 197, 198, 199, 200, 201, 202, 243.2014, 205, 206, 207, 208, 209, 210, 211, 212, 213, 214, 215, 216, 217, 218, 219, 220, 221, 222, 223, 224, 225, 226, 227, 228, 229, 230, 231, 232, 233, 234, 235, 236, 237, 238, 239, 240, 241, 242, 243, 244, 245, 246, 247, 248, 249, 250, 251, 252, 253, 254, 255, 256, 257, 258, 59, 260, 271, 272, 273, 274, 275, 276, 277, 278, 279, 280, 281, 282, 283, 284, 285, 286, 287, 288, 289, 290, 291, 292, 293, 294, 295, 296, 297, 298, 299, 300, 301, 302, 303, 304, 305, 306, 307, 308, 309, 310, 310, 311, 312, 313, 314, 315, 316, 317, 318, 319, 320, 321, 322, 323, 324, 325, 326, 327, 328, 329, 330, 331, 332, 333, 334, 335, 336, 337, 338, 339, 340, 341, 342, 343, 344, 345, 346, 347, 348, 349, 350, 351, 352, 353, 354, 355, 356, 357, 358, 359, 360, 361, 362, 363, 364, 365, 366, 367, 368, 369, 370, 371, 372, 373, 374, 375, 376, 377, 378, 379, 380, 381, 382, 383, 384, 385, 386, 387, 388, 389, 390, 391, 392, 393, 394, 395, 396, 397, 398, 399, 400, 401, 402, 403, 404, 405,406, 407, 408, 409, 410, 411, 412, 413, 414,415, 416, 417, 418, 419, 420, 421, 422, 423, 424, 425, 426, 427, 428, 429, 430, 431, 432, 433, 434, 435, 436, 437, 438, 439, 440, 441, 442, 443, 444, 445, 446, 447, 448, 449, 450, 451, 452, 453, 454, 455, 456, 457, 458, 459, 460, 461, 462, 463, 464, 465, 466, 467, 468, 469, 470, 471, 472, 473, 474, 475, 476, 477, 478, 479, 480, 481, 482, 483, 484, 485, 486, 487, 488, 489, 490, 491, 492, 493, 494, 495, 496, 497, 498, 499, 500, 501, 502, 503, 504, 505, 506, 507, 508, 509, 510, 511, 512, 513, 514, 515, 516, 517, 518, 519, 520, 521, 522, 523, 524, 525, 526, 527, 528, 529, 530, 531, 532, 533, 534, 535, 536, 537, 538, 539, 540, 541, 542, 543, 544, 545, 546, 547, 548, 549, 550, 551, 552, 553, 554, 555, 556, 557, 558, 559, 560, 561, 562, 563, 564, 565, 566, 567, 568, 569, 570, 571, 572, 573, 574, 575, 576, 577, 578, 579, 580, 581, 582, 583, 584, and 585 in SEQ ID NO: 13, wherein the N-terminal first residue will not be higher numbered than N−L+1, where N is the number of amino acid residues of the SEQ ID No: 13 and L is the number of amino acids defined for option b in claim 1 or 2.

5. The pharmaceutical composition according to claim wherein the polypeptide is fused or conjugated to an immunogenic carrier molecule.

6. The pharmaceutical composition according to claim 5, wherein the immunogenic carrier molecule is a polypeptide that induces T-helper lymphocyte responses in a majority of humans, such as immunogenic carrier proteins selected from the group consisting of keyhole limpet hemocyanin or a fragment thereof, tetanus toxoid or a fragment thereof, dipththeria toxoid or a fragment thereof.

7. The pharmaceutical composition according to claim 1, wherein the polypeptide is capable of inducing, in the mammal, a protective adaptive humoral or cellular immune response against infection with S. aureus.

8. The pharmaceutical composition according to claim 1, wherein the immunological adjuvant is an aluminium based adjuvant.

9. A method for inducing immunity in an animal by administering at least once to the animal an immunogenically effective amount of the pharmaceutical composition according to claim 1 or of the polypeptide defined in claim 1, so as to induce adaptive immunity against S. aureus in the animal.

10. The method according to claim 9, wherein the animal receives between 0.5 and 5,000 μg of the polypeptide according defined in claim 1 per administration.

11. The method according to claim 9, wherein the animal receives a priming administration and one or more booster administrations.

12. The method according claim 9, wherein the animal is a human being.

13. The method according to claim 9, wherein the administration is for the purpose of inducing protective immunity against S. aureus.

14. The method according to claim 13, wherein the protective immunity is effective in reducing the risk of attracting infection with S. aureus or is effective in treating or ameliorating infection with S. aureus.


SEQUENCE LISTING (53 sequences)

SEQ ID NO: 1 - protein, 140 aa, Staphylococcus aureus
MAKGNLFKAILGIGGAVAAVLVTRKDSRDKLKAEYNKYKQDPQSYKDNAKDKATQLGTIANETIKEVKTNPKEYANRLKNNPKAFFEEEKSKFTEYDNKTDESIEKGKFDDEGGAAPNNNLRIVTEEDLKKNKNALSDKE

SEQ ID NO: 2 - protein, 193 aa, Staphylococcus aureus
MKKLVSIVGATLLLAGCGSQNLAPLEEKTTDLREDNHQLKLDIQELNQQISDSKSKIKGLEKDKENSKKTASNNTKIKLMNVTSTYYDKVAKALKSYNDIEKDVSKNKGDKNVQSKLNQISNDIQSAHTSYKDAIDGLSLSDDDKKTSKNIDKLNSDLNHAFDDIKNGYQNKDKKQLTKGQQALSKLNLNAKS

SEQ ID NO: 3 - protein, 209 aa, Staphylococcus aureus
MKKLVTGLLALSLFLAACGQDSDQQKDSNKEKDDKAKTEQQDKKTNDSSKDKKDNKDDSKDVNKDNKDNSANDNQQQSNSNATNNDQNQTNNNQSNSGQTTNNQKSSYVAPYYGQNAAPVARQIYPFNGNKSQALQQLPNFQTALNAANNEANKFGNGHKVYNDYSIEEHNGNYKYVFSFKDPNVNGKYSIVTVDYTGQAMVTDPNYQQ

SEQ ID NO: 4 - protein, 242 aa, Staphylococcus aureus
MKKVMGILLASTLILGACGHHQDSAKKESTSHKKKENDNEELNEELKEFKSKKNMDIKIKGDTIVSDKFEAKIKEPFIINEKDEKKKYIAFKMEITAKKDDKDLNPSSISHDYINITQDDKNTVNKLRDGYLLSDKNYKDWTEHNQDQIKKGKTAQAMFIYELRGDGNINLNVHKYSEDKTVDSKSFKFSKLKTEDFSHRAETREEVEKKEKEFEEEYKKEQEREKEKEKQKDDDHSGLDEV

SEQ ID NO: 5 - protein, 327 aa, Staphylococcus aureus
MKKWQFVGTTALGATLLLGACGGGNGGSGNSDLKGEAKGDGSSTVAPIVEKLNEKWAQDHSDAKISAGQAGTGAGFQKFIAGDIDFADASRPIKDEEKQKLQDKNIKYKEFKIAQDGVTVAVNKENDFVDELDKQQLKAIYSGKAKTWKDVNSKWPDKKINAVSPNSSHGTYDFFENEVMNKEDIKAEKNADTNAIVSSVTKNKEGIGYFGYNFYVQNKDKLKEVKIKDENGKATEPTKKTIQDNSYALSRPLFIYVNEKALKDNKVMSEFIKFVLEDKGKAAEEGGYVAAPEKTYKSQLDDLKAFIDKNQKSDDKKSDDKKSEDKK

SEQ ID NO: 6 - protein, 357 aa, Staphylococcus aureus
MKGKFLKVSSLFVATLTTATLVSSPAANALSSKAMDNHPQQTQTDKQQTPKIQKGGNLKPLEQRERANVILPNNDRHQITDTTNGHYAPVTYIQVEAPTGTFIASGVVVGKDTLLTNKHIVDATHGDPHALKAFASAINQDNYPNGGFTAEQITKYSGEGDLAIVKFSPNEQNKHIGEVVKPATMSNNAETQVNQNITVTGYPGDKPVATMWESKGKITYLKGEAMQYDLSTTGGNSGSPVFNEKNEVIGIHWGGVPNQFNGAVFINENVRNFLKQNIEDINFANDDHPNNPDNPDNPNNPDNPNNPDNPNNPDNPDNPNNPDNPNNPDNPNNPDQPNNPNNPDNGDNNNSDNPDAA

SEQ ID NO: 7 - protein, 399 aa, Staphylococcus aureus
MKRTLVLLITAIFILAACGNHKDDQAGKDNQKHNNSSNQVKEIATDKNVQGDNYRTLLPFKESQARGLLQDNMANSYNGGDFEDGLLNLSKEVFPTDKYLYQDGQFLDKKTINAYLNLKYTKREIDKMSEKDKKDKKANENLGLNPSHEGETDPEKIAEKSPAYLSNILEQDFYGGGDTKGKNIKGMTIGLAMNSVYYYKKEKDGPTFSKKLDDSEVKKQGKQMASEILSRLRENDDLKDIPIHFAIYKQSSEDSITPGEFITQATAEKSQTKLNEWHNINEKSALLPSSTAADYDENLNNNFKQFNDNLQSYFSNFTQAVGKVKFVDKKPQRLVVDLPIDYYGQAETIGITQYVTEQANKYFDKIDNYEIRIKDGNQPRALISKTKDDKEPQVHIYSN

SEQ ID NO: 8 - protein, 495 aa, Staphylococcus aureus
MRENFKLRKMKVGLVSVAITMLYIMTNGQAEASEANEKPSTNQESKVVSQTEQNSKETKTVESNKNFVKLDTIKPGAQKITGTTLPNHYVLLTVDGKSADSVENGGLGFVEANDKGEFEYPLNNRKIVHNQEIEVSSSSPDLGEDEEDEEVEEASTDKAGVEEESTEAKVTYTTPRYEKAYEIPKEQLKEKDGHHQVFIEPITEGSGIIKGHTSVKGKVALSINNKFINFEESVKGGVSKEDTKASSDGIWMPIDDKGYFNFDFKTKRFDNLELKEGNDISLTFAPDDEEDALKPLIFKTKVTSLEDIDKAETKYDHTKLNKVKVLDNVKEDLHVDEIYGSLYHTDKGKGILDKEGTKVIKGKTKFANAVVKVDSELGEAQLFPDLQVNEKGEFSFDSHGAGFRLQNGEKLNFTVVDPITGDLLSNEFVSKEIDIEETPEQKADREFDEKLENTPAYYKLYGDKIVGFDTNDFPITWFYPLGEKKVERTTPKLEK

SEQ ID NO: 9 - protein, 507 aa, Staphylococcus aureus
MSKKLKIIIPIIIVLLLIGGIAWGVYAFFANTPKNTYLKSEQQTAKMYKDYFNDRFENEVKFQEKMKDNSFLSSLELSADASDEIVKGLGIPKSVVNASKIKMSYGHDPKKEKSMINLEPTIADSALGKFQLAADKDKHYFESPLFKGKYSVNNSDLLSTYSKLTGEDEETAKENGITNQQLNLNTLFSNAQAQQSDYSKIAEKYSELIVDKLDDDNFDKGKKEEIKVNGEKYKVRPVTLTLSRADTKKITLAVLEEAKKDKDLKKLMEEQGTTKDFEKDIKKAIDDVKETKKDEFAKIQSKIYTEKHTIVKREITITDKENNKTKIKGTNTLEDDKLKLDYALDFDQDKYTYAEAKYTIKGVSSKEKDNKYSDKYEFGKKTEYDESKIKLDNQEKVDGTKRQDKGKITVALDKYSDENEFTFENNIDSDVKNNTQKSTLNIGIKYAEEPINFILKSSTKLKADIDFDDSGAKDFNSLSSKDREKLEKEIEKNGGKMFESILKKASK

SEQ ID NO: 10 - protein, 509 aa, Staphylococcus aureus
MRKFSRYAFTSMATVTLLSSLTPAALASDTNHKPATSDINFEITQKSDAVKALKELPKSENVKNHYQDYSVTDVKTDKKGFTHYTLQPSVDGVHAPDKEVKVHADKSGKVVLINGDTDAKKVKPTNKVTLSKDEAADKAFNAVKIDKNKAKNLQDDVIKENKVEIDGDSNKYIYNIELITVTPEISHWKVKIDADTGAVVEKTNLVKEAAATGTGKGVLGDTKDININSIDGGFSLEDLTHQGKLSAYNFNDQTGQATLITNEDENFVKDDQRAGVDANYYAKQTYDYYKNTFGRESYDNHGSPIVSLTHVNHYGGQDNRNNAAWIGDKMIYGDGDGRTFTNLSGANDVVAHELTHGVTQETANLEYKDQSGALNESFSDVFGYFVDDEDFLMGEDVYTPGKEGDALRSMSNPEQFGQPSHMKDYVYTEKDNGGVHTNSGIPNKAAYNVIQAIGKSKSEQIYYRALTEYLTSNSNFKDCKDALYQAAKDLYDEQTAEQVYEAWNEVGVE

SEQ ID NO: 11 - protein, 515 aa, Staphylococcus aureus
MKKKLGMLLLVPAVTLSLAACGNDDGKDKDGKVTIKTTVYPLQSFAEQIGGKHVKVSSIYPAGTDLHSYEPTQKDILSASKSDLFMYTGDNLDPVAKKVASTIKDKDKKLSLEDKLDKAKLLTDQHEHGEEHEHEGHDHGKEEHHHHGGYDPHVWLDPKINQTFAKEIKDELVKKDPKHKDDYEKNYKKLNDDLKKIDNDMKQVTKDKQGNAVFISHESIGYLADRYGFVQKGIQNMNAEDPSQKELTKIVKEIRDSNAKYILYEDNVANKVTETIRKETDAKPLKFYNMESLNKEQQKKDNITYQSLMKSNIENIGKALDSGVKVKDDKAESKHDKAISDGYFKDEQVKDRELSDYAGEWQSVYPYLKDGTLDEVMEHKAENDPKKSAKDLKAYYDKGYKTDITNIDIKGNEITFTKDGTKHTGKYEYNGKKTLKYPKGNRGVRFMFKLVDGNDKDLPKFIQFSDHNIAPKKAEHFHIFMGNDNDALLKEMDNWPTYYPSKLNKDQIKEEMLAH

SEQ ID NO: 12 - protein, 564 aa, Staphylococcus aureus
MVLYIILAIIVIILIAVGVLFYLRSNKRQIIEKAIERKNEIETLPFDQNLAQLSKLNLKGETKTKYDAMKKDNVESTNKYLAPVEEKIHNAEALLDKFSFNASQCEIDDANELMDSYEQSYQQQLEDVNEIIALYKDNDELYDKCKVDYREMKRDVLANRHQFGEAASLLETEIEKFEPRLEQYEVLKADGNYVQAHNHIAALNEQMKQLRSYMEEIPELIRETQKELPGQFQDLKYGCRDLKVEGYDLDHVKVDSTLQSLKTELSFVEPLISRLELEEANDKLANINDKLDDMYDLIEHEVKAKNDVEETKDIITDNLFKAKDMNYTLQTEIEYVRENYYINESDAQSVRQFENEIQSLISVYDDILKEMSKSAVRYSEVQDNLQYLEDHVTVINDKQEKLQNHLIQLREDEAEAEDNLLRVQSKKEEVYRRLLASNLTSVPERFIIMKNEIDHEVRDVNEQFSERPIHVKQLKDKVSKIVIQMNTFEDEANDVLVNAVYAEKLIQYGNRYRKDYSNVDKSLNEAERLFKNNRYKRAIEIAEQALESVEPGVTKHIEEEVIKQ

SEQ ID NO: 13 - protein, 619 aa, Staphylococcus aureus
MPKNKILIYLLSTTLVLPTLVSPTAYADTPQKDTTAKTTSHDSKKSTDDETSKDTTSKDIDKADNNNTSNQDNNDKKVKTIDDSTSDSNNIIDFIYKNLPQTNINQLLTKNKYDDNYSLTTLIQNLFNLNSDISDYEQPRNGEKSTNDSNKNSDNSIKNDTDTQSSKQDKADNQKAPKSNNTKPSTSNKQPNSPKPTQPNQSNSQPASDDKVNQKSSSKDNQSMSDSALDSILDQYSEDAKKTQKDYASQSKKDKNEKSNTKNPQLPTQDELKHKSKPAQSFNNDVNQKDTRATSLFETDPSISNNDDSGQFNVVDSKDTRQFVKSIAKDAHRIGQDNDIYASVMIAQAILESDSGRSALAKSPNHNLFGIKGAFEGNSVPFNTLEADGNQLYSINAGFRKYPSTKESLKDYSDLIKNGIDGNRTIYKPTWKSEADSYKDATSHLSKTYATDPNYAKKLNSIIKHYQLTQFDDERMPDLDKYERSIKDYDDSSDEFKPFREVSDNMPYPHGQCTWYVYNRMKQFGTSISGDLGDAHNWNNRAQYRDYQVSHTPKRHAAVVFEAGQFGADQHYGHVAFVEKVNSDGSIVISESNVKGLGIISHRTINAAAAEELSYITGK

SEQ ID NO: 14 - protein, 681 aa, Staphylococcus aureus
MMKSQNKYSIRKFSVGASSILIATLLFLSGGQAQAAEKQVNMGNSQEDTVTAQSIGDQQTRENANYQRENGVDEQQHTENLTKNLHNDKTISEENHRKTDDLNKDQLKDDKKSSLNNKNIQRDTTKNNNANPRDVNQGLEQAINDGKQSKVASQQQSKEADNSQDLNANNNLPSQSRTKVSPSLNKSDQTSQREIVNETEIEKVQPQQKNQANDKITDHNFNNEQEVKPQKDEKTLSVSDLKNNQKSPVEPTKDNDKKNGLNLLKSSAVATLPNKGTKELTAKAKGDQTNKVAKQGQYKNQDPIVLVHGFNGFTDDINPSVLAHYWGGNKMNIRQDLEENGYKAYEASISAFGSNYDRAVELYYYIKGGRVDYGAAHAAKYGHERYGKTYEGIYKDWKPGQKVHLVGHSMGGQTIRQLEELLRNGSREEIEYQKKHSGEISPLFKGNNDNMISSITTLGTPHNGTHASDLAGNEALVRQIVFDIGKMFGNKNSRVDFGLAQWGLKQKPNESYIDYVKRVKQSNLWKSKDNGFYDLTREGATDLNRKTSLNPNIVYKTYTGEATHKALNSDRQKADLNMFFPFVITGNLIGKATEKEWRENDGLVSVISSQHPFNQAYTNATDKIQKGIWQVTPTKHDWDHVDFVGQDSSDTVRTREELQDFWHHLADDLVKTEKVTDTKQA

SEQ ID NO: 15 - protein, 806 aa, Staphylococcus aureus
MTNKMKKWQKLSTITLLMTGVIALNNGEFRNVDKHQIAVADTNVQTPDYEKLKKTWLDVNYGYDQYDENNQDMKKKFDAKEKEAKKLLDDMKTDTNRTYLWSGAENLETNSSHMTKTYRNIEKIAESMQHKNTVLKTVENKLKIKEALDWMHKNVYGKNPSQKVEDLTKNRKGQTTPKNNSLNWWDYEIGTPRALTNTLLLMDDMLTKDEMKNYSKPISTYAPSSDKILSSVGESEDAKGGNLVDISKVKLLESVIEEDVDMLKKSIDSFNKVFTYVQDSATGKGRNGFYKDGSYIDHQDVPYTGAYGVVLLEGISQMMPMIKESPFKTTQDNATLSNWIDEGFMPLIYKGEMMDLSRGRAISRENETSHTASATVMKSLLRLNDTMDDSTKTRYKQIVKTSVNSDSSYNQNNYLNSYSDIAKMKKLMNDSTISKNDLTQQLKIYNDMDRVTYHNKDLDFAFGLSMTSKNIARYENINGENLKGWHTGAGMSYLYNSDVKHYRDNFWATADMTCLPGTTTLNDMPSTNTKNDKSFVGGTKLNNKYASIGMDFENQDKTLTAKKSYFILNDKIVFLGTGIKSTDSSKNPVTSVENRKANGYKLFKDDIEITTSDVNAQETHSVFLESNDTKKNIGYHFLDKPKITVKKESHTGKWSEINKSQKKDDKKDEYYEVTQTHNTSDSKYAYVLYPGLSKSDFKSKNNNVSIVKQDEDFHVIKDNDGVFAGVNYSDNTKSFDINGITVELKEKGMFVIKKKDDKAYKCSFYNPETTNTASNIESKIFIKGYTITNKSVINSNDAGVNFELTK

SEQ ID NO: 16 - protein, 808 aa, Staphylococcus aureus
MTYRMKKWQKLSTITLLMAGVITLNGGEFRSIDKHQIAVADTNVQTTDYEKLRNIWLDVNYGYDKYDENNPDMKKKFEATENEAEKLLKEMKTESDRKYLWESSKDLDTKSADMTRTYRNIEKISEAMKHKNTKLKTDENKTKVKDALEWLHKNAYGKEPDKKVADLTSNFKNKTSRNTNLNWWDYEIGTPRALTNTLILLQEDFTDEEKKKYTAPIKTFAPDSDKILSSVGKSEPAKGGNLVDISKVKLLESIIEEDKDMMKKSIDSFNTVFTYAQNSATGKERNGFYKDGSYIDHQDVPYTGAYGVVLLEGISQMMPMIKETPFNDSNQNDTTLKSWIDDGFMPLIYKGEMMDLSRGRAISRENETSHSASATVMKSLLRLSDTMDKSTKAKYKKIVKTSVESDSSYKQTDYLSSYSDISKMKSLMEDSTISTNGLTQQLKIYNDMDRVTYHNKGLDFAFGLSMTSKNVARYESINGENLKGWHTGAGMSYLYNSDVKHYRDNFWATADMKRLAGTTTLDNEEPKSTDVKKSSKTFVGGTKFDDQHASIGMDFENQDKTLTAKKSYFILNDKIVFLGTGIKSTDSSKNPVTTIENRKANDYKLYKDDTQTTNSDNQETNSLFLESTNSTQNNIGYHFLNESKITVKKESHTGKWSDINKSQKDIQKTDEYYEVTQKHSNTDSKYAYVLYPGLSKDVFKSKASKVTVVKQEDDFHVVKDNESVWAGINYSDSAKTFEINNTKVEVKAKGMFILTKKDDNTYECSFYNPESTNSVSDIESKISMTGYSIINKNTSTSNESGVRFELTK

SEQ ID NO: 17 - DNA, 423 bp, Staphylococcus aureus
  source 1..423: mol_type = genomic DNA
atggcaaaaggtaatttatttaaagcgattttaggtataggtggcgctgtagcagctgtacttgttacacgtaaagatagtcgtgacaagctgaaagcagaatataataaatacaaacaagatcctcaaagctataaagataatgctaaggataaagcgacgcaattaggaacaattgcaaatgaaacaattaaagaagtaaaaacaaatccgaaagaatatgctaatagattaaaaaataatccaaaagcatttttcgaagaagaaaaatcaaaatttaccgaatatgacaataagactgacgaaagtattgaaaaaggtaaatttgatgatgaaggtggcgcagcaccaaataataatttacgtatcgtcactgaagaagatttaaaaaagaataaaaatgcattgtctgataaagaataa

SEQ ID NO: 18 - DNA, 582 bp, Staphylococcus aureus
  source 1..582: mol_type = genomic DNA
atgaaaaaattggtttcaattgttggcgcaacattattgttagctggatgtggatcacaaaatttagcaccattagaagaaaaaacaacagatttaagagaagataatcatcaactcaaactagatattcaagaacttaatcaacaaattagtgattctaaatctaaaattaaagggcttgaaaaggataaagaaaatagtaaaaaaactgcatctaataatacgaaaattaaattgatgaatgttacatcaacatactacgacaaagttgctaaagctttgaaatcctataacgatattgaaaaggatgtaagtaaaaacaaaggcgataagaatgttcaatcgaaattaaatcaaatttctaatgatattcaaagtgctcacacttcatacaaagatgctatcgatggtttatcacttagtgatgatgataaaaaaacgtctaaaaatatcgataaattaaactctgatttgaatcatgcatttgatgatattaaaaatggctatcaaaataaagataaaaaacaacttacaaaaggacaacaagcgttgtcaaaattaaacttaaatgcaaaatcatga

SEQ ID NO: 19 - DNA, 630 bp, Staphylococcus aureus
  source 1..630: mol_type = genomic DNA
atgaaaaaattagttacagggttattagcattatcattatttttagctgcatgtggtcaagatagtgaccaacaaaaagacagtaataaagaaaaagatgataaagcgaaaactgaacaacaagataaaaaaacaaatgattcatctaaagataagaaagacaataaagatgatagtaaagacgtaaacaaagataataaagataatagtgcaaacgataaccagcaacaatctaattcaaatgcaacaaacaatgaccaaaatcaaacgaataataaccagtcaaacagtggacaaacgactaacaatcaaaaatcaagttacgttgcaccatattatggacaaaacgcagcgccagtggctcgtcaaatttatccatttaatggtaataaatcacaagcattacaacaattgcctaatttccaaacagctttaaatgcagctaacaacgaagcaaataaatttggtaatggtcataaagtttataatgattattcaattgaagaacataatggtaactataagtatgtttttagttttaaagacccaaacgtaaatggaaaatattcaattgtaacggttgattatactggacaagcaatggttactgatccaaactaccaacaataa

SEQ ID NO: 20 - DNA, 729 bp, Staphylococcus aureus
  source 1..729: mol_type = genomic DNA
atgaaaaaagtaatggggatattattagcaagtacacttatcttaggtgcttgtggacatcatcaagatagtgcaaaaaaagagagcactagtcacaaaaagaaagaaaatgacaatgaagaattaaatgaagaacttaaagaatttaaaagcaaaaaaaatatggatataaaaattaaaggcgatactattgttagtgacaaatttgaagctaaaataaaagaaccgtttatcatcaatgaaaaagatgagaaaaagaaatatatcgcttttaaaatggaaattactgctaaaaaagacgataaagatttaaatccatcttctatttctcatgactatattaatatcactcaagatgataaaaatacagtaaataaattaagagatggttatcttttaagtgataaaaattataaagattggacagaacataaccaagatcaaattaaaaaaggcaaaactgcacaagccatgtttatctatgagttaagaggtgatggaaacattaatttaaatgtccataaatactcagaagataaaacagttgattctaaatcattcaaatttagtaaacttaaaaccgaagatttttctcatagagcggaaacaagggaagaagtagaaaagaaagaaaaagaatttgaagaagagtacaaaaaagaacaagaacgagagaaagaaaaagaaaagcaaaaagatgacgaccacagtggtttagatgaagtataa

SEQ ID NO: 21 - DNA, 984 bp, Staphylococcus aureus
  source 1..984: mol_type = genomic DNA
atgaaaaaatggcaatttgttggtactacagctttaggtgcaacactattattaggtgcttgtggtggcggtaatggtggcagtggtaatagtgatttaaaaggggaagctaaaggggatggctcatcaacagtagcaccaattgtggagaaattaaatgaaaaatgggctcaagatcactcggatgctaaaatctcagcaggacaagctggtacaggtgctggtttccaaaaattcattgcaggagatatcgacttcgctgatgcttctagaccaattaaagatgaagagaagcaaaaattacaagataagaatatcaaatacaaagaattcaaaattgcgcaagatggtgtaacggttgctgtaaataaagaaaatgattttgtagatgaattagacaaacagcaattaaaagcaatttattctggaaaagctaaaacatggaaagatgttaatagtaaatggccagataaaaaaataaatgctgtatcaccaaactcaagtcatggtacttatgacttctttgaaaatgaagtaatgaataaagaagatattaaagcagaaaaaaatgctgatacaaatgctatcgtttcttctgtaacgaaaaacaaagagggaatcggatactttggatataacttctacgtacaaaataaagataaattaaaagaagttaaaatcaaagatgaaaatggtaaagcaacagagcctacgaaaaaaacaattcaagataactcttatgcattaagtagaccattattcatttatgtaaatgaaaaagcattgaaagataataaagtaatgtcagaatttatcaaattcgtcttagaagataaaggtaaagcagctgaagaaggtggatatgtagcagcaccagagaaaacatacaaatcacaattagatgatttaaaagcatttattgataaaaatcaaaaatcagacgacaagaaatctgatgataaaaagtctgaagacaagaaataa

SEQ ID NO: 22 - DNA, 1074 bp, Staphylococcus aureus
  source 1..1074: mol_type = genomic DNA
atgaaaggtaaatttttaaaagttagttctttattcgttgcaactttgacaacagcgacacttgtgagttctccagcagcaaatgcgttatcttcaaaagctatggacaatcatccacaacaaacgcagacagacaaacagcaaacacctaagattcaaaaaggcggtaaccttaaaccattagaacaacgtgaacgcgctaatgttatattaccaaataacgatcgtcaccaaatcacagatacaacgaatggtcattatgcacctgttacttatattcaagttgaagcacctactggtacatttattgcttctggtgtagttgtaggtaaagatacacttttaacaaataaacacatcgtagatgctacgcacggtgatcctcatgctttaaaagcattcgcttctgcaattaaccaagacaattatcctaatggtggtttcactgctgaacaaatcactaaatattcaggcgaaggtgatttagcaatcgttaaattctcccctaatgagcaaaacaaacatattggcgaagtagttaaaccagcaacaatgagtaataatgctgaaacacaagttaaccaaaatattactgtaacaggatatcctggtgataaacctgtcgcaacaatgtgggaaagtaaaggaaaaataacgtacttaaaaggtgaagcaatgcaatatgatttaagtacaactggtggtaactcaggttcacctgtatttaatgaaaaaaatgaagtcattggcattcattggggtggcgttccaaatcaatttaacggtgcagtatttattaatgaaaatgtacgcaacttcttaaaacaaaatattgaagatatcaatttcgcaaatgatgaccaccctaacaaccctgataatccagacaatccaaataatccggacaatcctaacaaccctgataaccctaacaaccctgataatccagacaatcctaataatcctgataaccctaacaacccggacaatccaaataaccctgaccaacctaacaacccaaataacccggacaatggcgataacaataattcagacaaccctgacgctgcataa

SEQ ID NO: 23 - DNA, 1200 bp, Staphylococcus aureus
  source 1..1200: mol_type = genomic DNA
atgaagcgtacattagtattattgattacagctatctttatactcgctgcttgtggtaaccataaggatgaccaggctggaaaagataatcaaaaacataacaatagttcaaatcaagtaaaagaaattgctacggataaaaatgtacaaggtgataactatcgtacattgttaccatttaaagaaagccaggcaagaggacttttacaagataacatggcaaatagttataatggcggcgactttgaagatggtttattgaacttaagtaaagaagtgtttccaacagacaaatatttgtatcaagatggtcaatttttggacaagaaaacaattaatgcctatttaaatcttaagtatacaaaacgtgaaatcgataaaatgtctgaaaaagataaaaaagacaagaaagcgaatgaaaatttaggacttaatccatcacacgaaggtgaaacagatcctgaaaagattgcagaaaaatcaccagcctatttatctaacattttagagcaagatttttatggtggtggagatacaaaaggtaagaatattaaaggtatgacgattggtttagctatgaatagtgtttattactataaaaaagaaaaagatggaccgacttttagtaaaaaactagatgatagcgaagttaaaaagcaaggtaaacaaatggctagtgagatattatcaaggttacgtgaaaatgatgatttaaaagatataccaattcattttgcaatttataagcaatcaagtgaagattcaatcacaccaggtgaatttatcactcaagcgactgcagaaaagagtcaaacaaagcttaatgaatggcataatatcaatgaaaaatcagctttattaccttcttcaacagcagcagattatgatgaaaatttaaataataatttcaagcaatttaatgataatttgcaatcatatttttctaatttcacacaagcagtaggaaaagttaaatttgttgataaaaagccacaacgattagtagtagatttaccaatcgattactatggacaagctgaaacaattggtattacacagtacgttactgaacaagcgaataaatatttcgataaaatcgataactatgaaattcggattaaagatggtaaccaaccacgtgctttaattagtaagacaaaagatgacaaagaaccgcaagttcatatttacagtaattaa

SEQ ID NO: 24 - DNA, 1488 bp, Staphylococcus aureus
  source 1..1488: mol_type = genomic DNA
atgagggaaaattttaagttacgtaaaatgaaagtcgggttagtatctgttgcaattacaatgttatatatcatgacaaacggacaagcagaagcatcagaggctaatgagaagccaagtacaaatcaagaatcaaaagttgtttcacagactgaacaaaattcaaaagaaacaaaaacagtagaatctaataagaactttgttaaattagatactattaaacctggagctcaaaagataacgggaactactttaccaaatcactatgttttattaacagttgatgggaaaagtgcggattcagtagaaaatggcggtttgggttttgttgaagcaaatgacaaaggagaatttgagtaccctttaaataatcgtaaaattgttcataatcaagaaattgaggtttcgtcgtcaagccctgatttaggtgaagatgaagaagatgaagaggtggaagaagcttcaactgataaagctggcgttgaggaagaaagtacagaagctaaagttacttacacaacaccgcgatatgaaaaagcgtatgaaataccgaaagaacaactaaaagaaaaagatggacatcaccaagtttttatcgaacctattactgaaggatcaggtattattaaagggcatacgtctgtaaaaggtaaagttgctttatctattaataataaatttattaattttgaagagagcgttaagggcggagttagtaaagaagacactaaagctagttcagatggtatctggatgcctattgatgacaaaggatactttaactttgacttcaaaacgaaacgtttcgataatttagagttaaaagaaggtaatgacatttcactaacatttgcacctgatgatgaagaagatgcattaaaacctttaattttcaaaactaaagtaacgagcttagaagatatcgataaagcagaaactaaatatgaccatactaaactcaacaaagtgaaagttttagataatgttaaagaagatttacatgttgatgaaatatatggaagcttatatcatacagacaaaggtaaaggtattcttgataaagaaggtactaaagtaattaaaggaaagactaaattcgcgaatgcagtagtgaaggtagactctgaactaggtgaagcacaattattccctgatttacaagtaaatgaaaaaggtgaatttagctttgactcacatggtgctggttttagattacaaaatggagaaaaattaaacttcacagtggttgatcctattacaggtgacttgttaagtaatgagtttgtttctaaagagattgatattgaagaaacacctgaacaaaaagcggatcgtgagtttgacgaaaaacttgaaaatacgcctgcttactacaagttatacggcgataaaatagttggattcgatactaacgatttcccgattacttggttctatccattgggtgaaaagaaagttgaacgtacaacacctaaattagaaaaataa

SEQ ID NO: 25 - DNA, 1524 bp, Staphylococcus aureus
  source 1..1524: mol_type = genomic DNA
atgtctaaaaagttaaaaattataattcctattattattgtcttattattaataggtggaatcgcatggggagtttatgcattttttgcaaacacaccgaaaaatacatacttaaaaagtgaacaacaaactgcaaaaatgtataaagattattttaatgaccgttttgaaaacgaagtgaagttccaagaaaagatgaaagataattcatttttatcttcattagaattaagcgcagatgcatctgatgaaattgttaaagggcttggtattcctaaatctgttgttaatgcttcgaaaattaaaatgtcatatggacatgatcctaaaaaagagaaatcaatgattaatcttgaaccaacaatagcagactctgcattagggaaattccagttagctgcagataaagataagcattatttcgaatcaccattatttaaagggaaatatagtgttaataattctgatttattatcaacttattcaaaacttacaggtgaagatgaagaaacagcaaaagaaaatggtattacaaaccaacaactaaatttaaatactcttttcagtaatgctcaagcacaacaaagtgactacagcaaaattgccgaaaaatattccgaacttattgtcgacaaattagatgacgataattttgataaaggtaaaaaagaagaaattaaggttaatggtgaaaagtacaaagttagacctgtcacgttaacacttagcagagctgacactaaaaaaattacattagctgtattagaagaagctaaaaaggataaagaccttaaaaaattaatggaagaacaaggtactacaaaagactttgaaaaagacattaaaaaagcaattgacgatgtcaaagaaactaaaaaggatgaatttgctaaaattcaatctaaaatttataccgaaaaacatacgattgtaaaacgagaaattactattacagacaaagaaaataataaaactaaaatcaaaggtactaatactttagaagacgataagttaaaactagattacgcacttgatttcgatcaagataaatacacgtatgctgaagcgaaatatacaattaaaggcgtatcttctaaggaaaaagacaataaatacagtgataaatacgaatttggtaaaaagacagaatatgatgaatcaaaaatcaaattagataaccaagaaaaagtagatggcacaaaacgtcaagataaaggtaaaatcactgtcgcgttagataaatatagcgacgaaaatgaattcacttttgaaaataatatagattctgacgtaaaaaataacactcagaaatctacgttaaatatcggcatcaaatatgctgaagaaccaattaatttcattttaaaatctagcacaaaattgaaagcagatattgattttgatgatagtggtgcgaaagatttcaatagtctatcttcaaaagaccgtgaaaaacttgaaaaagaaatcgaaaaaaatggcggcaaaatgtttgaatcaattttaaaaaaggcatctaaataa

SEQ ID NO: 26 - DNA, 1530 bp, Staphylococcus aureus
  source 1..1530: mol_type = genomic DNA
gtgaggaaattttcaagatatgcatttacaagtatggcaacagtaacgttgctgagctctttgacacctgcagcactagcgagtgatacgaatcacaaaccagcaacttcagatattaattttgaaatcacgcaaaagagtgatgcagttaaagcattaaaagagttacctaaatctgaaaatgtgaaaaatcattatcaagattactctgttacagatgtaaaaacagataagaaaggattcacgcattacacgttacaaccgagtgtggatggtgtgcatgcgcctgacaaagaagtgaaagtgcatgcggacaaatcgggtaaagtcgttttaatcaacggtgatactgatgcgaagaaagtaaagccgacaaataaagtgacattaagcaaggatgaagcggctgacaaagcatttaacgcagttaagattgataaaaataaagctaaaaacctccaagatgacgttatcaaagaaaataaagtcgaaatcgatggtgacagtaataaatacatttacaatattgaattaattacagtaacaccagaaatttcacattggaaagttaaaattgatgcagacacaggagcagttgttgaaaaaacgaacttagttaaagaagcagcagcaactggcacaggtaaaggtgtgcttggagatacaaaagatatcaatatcaatagtattgatggtggatttagtttagaggatttgacgcatcaaggtaaattatcagcatacaattttaacgatcaaacaggtcaagcgacattaattactaatgaagatgaaaacttcgtcaaagatgatcaacgtgctggtgtagatgcgaattattatgctaaacaaacatatgattactacaaaaatacatttggtcgtgagtcttacgataaccatggtagtccaatagtctcattaacacatgtaaatcattatggtggacaagataacagaaataacgctgcatggattggagacaaaatgatttatggtgatggcgatggccgcacgtttacaaatttatcaggtgcaaatgacgtagtagcacatgagttaacacatggcgtgacacaagaaacggcgaatttagagtataaagatcaatctggtgcgttaaatgaaagcttttcagatgtttttggatactttgtagatgatgaggatttcttgatgggtgaagatgtttacacaccaggaaaagagggagatgctttacgaagcatgtcaaacccagaacaatttggtcaaccatctcatatgaaagactatgtatacactgaaaaagataacggtggtgtgcatacgaattctggcattccaaataaagcagcttataacgtaattcaagcaatagggaaatctaaatcagaacaaatttactaccgagcattaacggaatacttaacaagtaattcaaacttcaaagattgtaaagatgcattataccaagcggctaaagatttatatgacgagcaaacagctgaacaagtatatgaagcatggaacgaagttggcgtcgagtaa

SEQ ID NO: 27 - DNA, 1548 bp, Staphylococcus aureus
  source 1..1548: mol_type = genomic DNA
atgaaaaagaaattaggtatgttacttcttgtaccagccgtaactttatcattagccgcatgtgggaatgatgatggaaaagataaagatggcaaggtaacaattaaaacgacagtttatccattgcaatcatttgcagagcaaattggtggaaaacacgtgaaggtatcatcaatctatccagcagggacagatttacatagctatgaaccaacacaaaaagatatattaagtgcaagcaagtcagacttgtttatgtatacaggggataatttagatccggttgctaagaaagttgcatctactattaaagataaagataaaaaactgtctttagaagataaattagataaagcaaagcttttaactgatcaacacgaacatggtgaagagcatgaacatgagggacatgatcatgggaaagaagaacatcatcatcatggcggatatgatccacacgtatggttagatcctaaaattaaccaaactttcgctaaagaaattaaagatgaattagtgaagaaagatccaaaacataaagatgactatgagaaaaactacaaaaaattaaacgacgatcttaagaaaattgataacgatatgaagcaagttactaaagataagcaaggtaatgcagtattcatttcacatgaatcaattggatacttagctgatcgttatggttttgttcaaaaaggtattcaaaacatgaatgctgaagatccatcacaaaaagaattgactaaaattgttaaagaaattagagatagcaatgctaaatatattctttacgaagataatgttgcgaataaagtgactgaaacaattcgtaaagaaacagatgcgaagcctttaaaattctacaacatggagtctttaaataaagaacaacagaaaaaagataatattacctatcaatcattaatgaaatcgaatattgaaaatatcggtaaagctttagacagtggtgttaaagtgaaagacgacaaagctgaaagtaaacacgacaaagcaatttctgatgggtattttaaagatgagcaagttaaagaccgtgaattaagcgattatgctggtgaatggcaatctgtttacccttacttaaaagacggtacgcttgatgaagtgatggaacataaagctgaaaatgatccgaagaaatctgctaaagatttaaaagcttattatgacaaaggatataaaactgatattactaacattgatataaaaggaaatgaaattacatttactaaagatggtacgaaacacactggtaaatatgaatacaatggtaagaaaacattgaaatatcctaaaggtaaccgtggcgtgagatttatgtttaaattggtcgatggtaatgataaagacttaccgaaattcatccaatttagcgatcacaacattgcacctaaaaaggcagaacacttccatatctttatgggtaatgataatgacgcgttattaaaagaaatggataactggccaacatattatccttcaaaattaaataaagaccaaatcaaagaagaaatgttagcgcattaa

SEQ ID NO: 28 - DNA, 1695 bp, Staphylococcus aureus
  source 1..1695: mol_type = genomic DNA
atggtgttatatatcattttggcaataattgtgattatattgattgctgtaggtgtattattctatttacgttcaaataaaagacaaataatagaaaaagcaatcgaacgtaaaaatgaaattgaaacgttaccttttgatcaaaaccttgcacaattatctaagttgaatttaaaaggtgaaacaaaaacgaaatacgatgcaatgaaaaaggacaacgtagaaagtacaaataagtatctagctcctgtggaagaaaaaatccataatgctgaggctttattagataaatttagtttcaacgcatctcaatgtgaaattgatgatgcaaatgagttgatggatagttacgaacaaagctatcagcaacaattagaagatgtaaatgaaattattgcgttatacaaagataatgatgaattatatgacaaatgtaaggttgattatcgtgaaatgaaacgtgatgttttagcaaatcgtcatcaatttggtgaggcagcaagtcttcttgaaactgaaattgaaaaattcgagccaaggttagagcaatatgaagtactaaaagctgatggtaattatgtacaagcgcacaaccatatagctgccttgaatgaacaaatgaaacagctaagatcttatatggaagaaataccagaattaattagagaaactcaaaaagaattacctggtcaattccaagatttaaaatatggttgccgtgatcttaaagttgaagggtatgatctggatcacgtaaaagtagacagtacattacaaagcttaaaaacagagcttagtttcgttgaaccattaattagtcgcttagaattagaagaagctaatgataaactagctaatatcaatgataagttagatgacatgtatgatttaattgaacatgaagttaaagctaaaaatgatgtcgaagaaacaaaagatatcattacggataacttattcaaagcaaaagacatgaattatacattgcaaacagaaattgaatatgtacgtgaaaactactatataaatgaatctgatgctcagagtgttcgtcaatttgaaaatgaaattcaaagtttaatttctgtatatgatgatattttaaaagaaatgtctaaatctgctgtacgatatagcgaggttcaggataatttacaatatttagaagatcatgtcacagttattaatgacaaacaagaaaagctacaaaatcatctgattcaattgcgtgaagatgaagcagaagcagaagacaatctgttacgagtacaatcgaagaaagaagaagtgtatcgtcgattacttgcttctaacttaacaagcgttcctgaaaggtttatcatcatgaaaaatgaaattgatcatgaagttcgtgatgttaacgaacaatttagtgaacgtccaatacacgttaaacagttaaaagataaagtgtctaaaattgtgattcaaatgaatacatttgaagatgaagcaaatgatgttcttgttaatgctgtttatgcagagaaattaattcaatatggaaatagatatcgtaaggactatagcaatgttgataagagcttaaatgaagctgaacgattatttaaaaataatcgctataagcgtgcgattgaaattgcagagcaagctcttgaaagtgttgagccaggtgtcactaaacatattgaagaagaagttattaagcaatag

SEQ ID NO: 29 - DNA, 1860 bp, Staphylococcus aureus
  source 1..1860: mol_type = genomic DNA
atgcctaaaaataaaattttaatttatttgctatcaactacgctcgtattacctactttagtttcacctaccgcttatgctgacacacctcaaaaagatactacagctaagacaacatctcatgattccaaaaaatctactgatgatgaaacttctaaggatactacaagtaaagatattgataaagcagacaacaataatactagtaaccaagacaataacgacaaaaaagttaaaactatagacgacagcacttcagactctaacaatatcattgattttatttataagaatttaccacaaaccaatataaaccaattgctaaccaaaaataaatacgatgataattactcattaacaactttaatccaaaacttattcaatttaaattcggatatttctgattacgaacaacctcgtaatggtgaaaagtcaacaaatgattcgaataaaaacagtgataatagcatcaaaaatgatacggatacgcaatcatctaaacaagataaagcagacaatcaaaaagcacctaaatcaaacaatacaaaaccaagtacatctaataagcaaccaaattcgccaaagccaacacaaccaaatcaatcaaatagtcaaccagcaagtgacgataaagtaaatcaaaaatcttcatcgaaagataatcaatcaatgtcagattcggctttagattctattttggatcaatacagtgaagatgcaaagaaaacacaaaaagattacgcatctcaatctaaaaaagacaaaaatgaaaaatctaatacaaagaatccacagttaccaacacaagatgaattgaaacataaatctaaacctgctcaatcattcaataacgatgttaatcaaaaggatacacgtgcaacatcactattcgaaacagatcctagtatatctaacaatgatgatagtggacaatttaacgttgttgactcaaaagatacacgtcaatttgtcaaatcaattgctaaagatgcacaccgcattggtcaagataacgatatttatgcgtctgtcatgattgcccaagcaatcttagaatctgactcaggtcgtagtgctttagctaagtcaccaaaccataatttattcggtatcaaaggtgcttttgaagggaattctgttccttttaacacattagaagctgatggtaatcaattgtatagtattaatgctggattccgaaaatatccaagcacgaaagaatcactaaaagattactctgaccttattaaaaatggtattgatggcaatcgaacaatttataaaccaacatggaaatcggaagccgattcttataaagatgcaacatcacacttatctaaaacatatgctacagatccaaactatgctaagaaattaaacagtattattaaacactatcaattaactcagtttgacgatgaacgtatgccagatttagataaatatgaacgttctatcaaggattatgatgattcatcagatgaattcaaacctttccgcgaggtatctgataatatgccatatccacatggccaatgtacttggtacgtatataaccgtatgaaacaatttggtacatctatctcaggtgatttaggtgatgcacataattggaataatcgagctcaataccgtgattatcaagtaagtcatacaccaaaacgtcatgctgctgttgtatttgaggctggacaatttggtgcagatcaacattacggtcatgtagcatttgttgaaaaagttaacagtgatggttctatcgttatttcagaatccaatgttaaaggattaggtatcatttctcatagaactatcaatgcagctgccgctgaagaattatcatatattacaggtaaataa

SEQ ID NO: 30 - DNA, 2046 bp, Staphylococcus aureus
  source 1..2046: mol_type = genomic DNA
atgatgaaaagtcaaaataagtatagtattcgtaaatttagtgtaggtgcatcttccattttaatagctacattactatttttaagtggtggacaagcacaagcagctgagaagcaagtgaatatgggaaattcacaggaggatacagttacagcacaatctattggggatcaacaaactagggaaaatgctaattatcaacgtgaaaacggtgttgacgaacagcaacatactgaaaatttaactaagaacttgcataatgataaaacaatatcagaagaaaatcatcgtaaaacagatgatttgaataaagatcaactaaaggatgataaaaaatcatcgcttaataataaaaatattcaacgtgatacaacaaaaaataacaatgctaatcctagggatgtaaatcaagggttagaacaggctattaatgatggcaaacaaagtaaagtggcgtcacagcaacagtcaaaagaggcagataatagtcaagacttaaacgctaataacaatctaccttcacaaagtcgaacaaaggtatcaccatcattaaataagtcagatcaaacaagtcaacgagaaattgttaatgagacagaaatagagaaagtacaaccgcaacaaaagaatcaagcgaatgataaaattactgaccacaattttaacaatgaacaagaagtgaaacctcaaaaagacgaaaaaacactatcagtttcagatttaaaaaacaatcaaaaatcaccagttgaaccaacaaaggacaatgacaagaaaaatggattaaatttattaaaaagtagtgcagtagcaacgttaccaaacaaagggacaaaggaacttactgcaaaagcgaaaggtgatcaaacgaataaagttgccaaacaagggcagtataaaaatcaagatcctatagttttagtgcatggtttcaatgggtttacagatgatattaatccttcagtgttagctcattattggggcggtaataaaatgaacattcgccaagatttagaagaaaatggttacaaagcttatgaagcaagtataagtgcttttggaagtaactatgaccgcgcagttgaactttattattatatcaaaggcggtcgtgtagattatggtgcagcacatgcagcaaaatatggacatgaacgttatggaaaaacatacgaaggaatttacaaagactggaaaccaggacagaaggtacaccttgttggacatagtatgggtggtcaaacgatacgtcaactagaagaattactgcgtaatggtagtcgtgaagaaatagagtatcaaaagaaacatagtggcgaaatttctccactattcaaaggtaataatgacaatatgatttcatcaattactactttaggaacgccacataatggaacgcatgcttcagatttagctggtaatgaagctttagtgagacaaattgtatttgatatcggtaaaatgtttggtaataaaaattcaagagtagacttcgggttggctcaatggggtctaaaacagaagccaaatgaatcatatattgattatgtcaaacgcgttaaacaatctaatttatggaaatcaaaagataatggattttacgatctgacgcgtgagggtgcaacagatttaaatcgtaaaacgtcgttgaaccctaacattgtgtataaaacatacactggtgaagcaacgcacaaagcattaaatagcgatagacaaaaagcagacttaaatatgtttttcccatttgtgattactggtaacttaatcggtaaagctactgaaaaagaatggcgagaaaacgatggtttagtatccgttatttcttctcaacatccatttaatcaagcttatacaaatgcgacggataaaattcaaaaaggcatttggcaagtaacgcctacaaaacatgattgggatcatgttgattttgtcggacaagatagttctgatacagtgcgcacaagagaagaattacaagatttttggcatcatttagcagacgatttagtgaaaactgaaaaggtgactgatactaagcaagcataa

SEQ ID NO: 31 - DNA, 2421 bp, Staphylococcus aureus
  source 1..2421: mol_type = genomic DNA
atgacaaataaaatgaagaaatggcaaaaattatccaccattacgttattaatgaccggagtgattgctttaaataatggtgaatttagaaatgttgataaacatcaaatcgctgtggctgatacgaatgttcaaacgccagattatgaaaaattgaagaagacgtggctcgacgttaactacggttatgatcagtatgatgagaataatcaagatatgaagaagaagtttgatgctaaagaaaaagaagccaagaagttacttgatgacatgaaaactgatacgaatagaacatatttgtggtcaggagctgaaaaccttgaaactaattcttctcacatgacaaaaacctatcgtaatatcgagaaaatcgcagaatcaatgcaacataagaatacggtattaaaaacagttgaaaacaagttgaaaataaaagaagccctagattggatgcacaaaaatgtttatggcaagaatccttctcaaaaagtcgaggatttaactaaaaatcgtaaggggcaaactacacccaagaataactcattgaattggtgggattatgaaattggtacgccaagagcattaacaaatacactacttctaatggatgatatgctcactaaagatgaaatgaaaaattattcaaaacctattagtacatatgcaccatccagtgacaaaattttatcttctgttggtgaatcagaagatgctaaaggtggaaatttagtggacatttctaaagtaaaacttttagaaagtgttattgaagaagatgtagatatgttgaaaaagtctatagattcttttaataaagtgttcacttatgttcaagattctgccactggtaaaggtcgcaatggattctataaagatggctcttacattgatcatcaagatgtcccttacactggtgcttatggtgttgtactattagagggtatttctcaaatgatgccgatgataaaagaatctccttttaaaactacacaagataatgctacattaagcaattggattgacgaagggtttatgccattaatctataaaggtgaaatgatggatttatcacgaggtagagctatcagtcgtgaaaatgaaacgagtcatacagcgtcagcgactgtaatgaaatcattgttgagattgaatgataccatggatgattcaacaaaaactagatataagcaaatcgttaaaacttctgttaattctgattcaagttacaaccaaaataattatttaaattcatattcagacatagctaaaatgaaaaagttaatgaatgatagtactatttctaaaaacgatttaacacagcaacttaaaatatataatgacatggatcgtgtcacctatcacaataaagacctggactttgcatttggtttaagtatgacatcgaaaaacatcgcacgatacgaaaatatcaacggagagaacttaaaaggttggcacaccggtgcaggcatgtcttatttatataacagcgatgtcaaacactatcgcgataacttctgggcaacagccgatatgacttgtcttccaggcactactactttaaatgatatgccatctactaatactaagaatgataaatcttttgttggcgggacaaaattaaataataaatacgcaagcatcggtatggattttgaaaatcaggacaaaactttaactgccaaaaaatcatatttcatattaaacgataaaattgtcttcttaggaactggcattaaaagtactgattcatcaaagaatccagttacaagtgttgaaaatcgcaaagcaaatgggtataaattatttaaagatgatattgaaattaccacttcagatgttaatgctcaggaaacccattcagtctttttagagtccaacgatactaaaaagaacattggttatcatttcttagacaagccaaaaataactgtaaaaaaagaaagtcatactggtaagtggagtgaaattaataaaagtcaaaaaaaagatgacaaaaaagatgagtattatgaagtaactcaaacacataatacatctgacagtaaatatgcatatgttttgtatcctggtttatcaaaaagtgattttaaatcgaagaataataatgtaagtattgttaaacaagatgaagattttcatgtgataaaagataatgatggcgtatttgctggggttaattatagtgataatactaaatcttttgatataaacggaattactgttgaattaaaagaaaaaggcatgtttgtaattaaaaagaaagatgataaagcatataaatgtagcttctataatcctgaaactacaaataccgcttcaaatatagaatcaaaaatttttattaaaggttacaccataactaataaaagtgtcataaactctaatgatgctggtgtaaactttgaattaactaaataa

SEQ ID NO: 32 - DNA, 2427 bp, Staphylococcus aureus
  source 1..2427: mol_type = genomic DNA
atgacatatagaatgaagaaatggcaaaaattgtccaccattacgttattaatggctggtgtgattactttgaatggtggtgaattcagaagtattgataaacatcaaatcgctgtggctgatacgaatgttcaaacgacagattatgaaaagttgaggaacatatggctggacgttaactatggttatgataagtatgatgagaataatccagatatgaagaagaagtttgaggctacggagaatgaggcagagaaattactcaaggaaatgaaaactgaaagtgataggaaatacttgtgggaaagctcaaaagatttagatacgaagtctgcggatatgactcgtacctatcgtaatattgagaaaatctcagaagcgatgaaacataaaaatactaaattaaaaacagatgaaaacaagacaaaagtaaaagatgcacttgagtggctgcataaaaatgcatatggaaaagaaccagataaaaaagttgctgatttgacctcaaactttaaaaataaaacttctagaaataccaacttaaattggtgggattatgaaattggaacacctagagcattaacaaatacgcttatactcttacaagaagatttcactgatgaagaaaagaaaaaatatacagctcctattaaaactttcgccccagatagtgacaaaatattatcttctgtaggaaaatctgaacctgctaaaggcggaaatttagtagacatttctaaagtaaaacttttagaaagtattatcgaagaagacaaagatatgatgaaaaagtctatagattcatttaatacagtcttcacttacgcgcaaaattctgccactggaaaagaacgtaatggattctataaagatggctcttacattgatcatcaagacgtcccatacactggtgcttatggcgttgtactattagagggtatttctcaaatgatgccgatgataaaagaaacaccttttaatgatagtaaccaaaatgatacaaccttaaaatcatggattgacgacggatttatgccactcatttataaaggtgaaatgatggatttatcaagaggtagagctatcagtcgtgaaaatgaaacgagtcactcagcatctgcaacagtaatgaaatcattgttgagattgagtgataccatggataagtctacaaaagctaaatataaaaagattgtcaagacttcagtagagtcagattcaagttataaacaaaccgattatttaagctcttattcggatataagcaaaatgaagtctttaatggaagacagcactatttctactaacggtttaacacaacaacttaaaatatataatgacatggatcgtgtcacctatcacaataaaggcttagactttgcatttggtttaagtatgacgtcgaaaaacgtcgcacgttacgaaagtatcaacggagagaacttaaaaggttggcacactggtgctggaatgtcttatttatacaatagcgatgtgaaacactaccgtgataacttctgggcgacagctgatatgaaacgtttagcaggtactacaactttagataatgaagaacctaaaagtacggatgttaaaaagtctagtaaaacttttgtaggaggaacaaaattcgatgaccaacatgctagtatcggaatggattttgaaaatcaggacaaaactttaactgccaaaaaatcatatttcatattaaacgataaaattgtcttcttaggaactggcattaaaagtactgattcatcaaagaatccagttacaacgattgaaaatcgcaaagcgaatgattataaattatataaagatgatacgcaaacaaccaattccgataatcaggaaaccaattccctctttttagagtcaacgaatagcactcaaaacaatataggttatcattttttaaacgaatcgaaaataactgtaaaaaaagaaagtcatactggtaagtggagtgatataaataaaagccaaaaggatatacaaaaaactgatgagtattatgaagtaactcaaaagcattctaatacagatagtaaatatgcatatgtgttgtatccaggcttatctaaagatgtctttaaatccaaagcaagcaaagtaactgtcgttaagcaagaagatgacttccacgttgtgaaagataatgaatcggtttgggctggtatcaattatagtgatagcgctaaaacttttgaaattaataacactaaagtcgaagttaaagccaaaggaatgtttattcttacaaagaaagatgataacacttatgaatgtagcttctataatcccgaatctacaaattccgtttcagatattgaatctaaaatttcaatgactggatactctattataaacaaaaatacgtcgacttctaatgaatccggcgtacgctttgaattaactaaataa

SEQ ID NO: 33 - RNA, 423 nt, Staphylococcus aureus
  source 1..423: mol_type = mRNA
atggcaaaaggtaatttatttaaagcgattttaggtataggtggcgctgtagcagctgtacttgttacacgtaaagatagtcgtgacaagctgaaagcagaatataataaatacaaacaagatcctcaaagctataaagataatgctaaggataaagcgacgcaattaggaacaattgcaaatgaaacaattaaagaagtaaaaacaaatccgaaagaatatgctaatagattaaaaaataatccaaaagcatttttcgaagaagaaaaatcaaaatttaccgaatatgacaataagactgacgaaagtattgaaaaaggtaaatttgatgatgaaggtggcgcagcaccaaataataatttacgtatcgtcactgaagaagatttaaaaaagaataaaaatgcattgtctgataaagaataa

SEQ ID NO: 34 - RNA, 582 nt, Staphylococcus aureus
  source 1..582: mol_type = mRNA
atgaaaaaattggtttcaattgttggcgcaacattattgttagctggatgtggatcacaaaatttagcaccattagaagaaaaaacaacagatttaagagaagataatcatcaactcaaactagatattcaagaacttaatcaacaaattagtgattctaaatctaaaattaaagggcttgaaaaggataaagaaaatagtaaaaaaactgcatctaataatacgaaaattaaattgatgaatgttacatcaacatactacgacaaagttgctaaagctttgaaatcctataacgatattgaaaaggatgtaagtaaaaacaaaggcgataagaatgttcaatcgaaattaaatcaaatttctaatgatattcaaagtgctcacacttcatacaaagatgctatcgatggtttatcacttagtgatgatgataaaaaaacgtctaaaaatatcgataaattaaactctgatttgaatcatgcatttgatgatattaaaaatggctatcaaaataaagataaaaaacaacttacaaaaggacaacaagcgttgtcaaaattaaacttaaatgcaaaatcatga

SEQ ID NO: 35 - RNA, 630 nt, Staphylococcus aureus
  source 1..630: mol_type = mRNA
atgaaaaaattagttacagggttattagcattatcattatttttagctgcatgtggtcaagatagtgaccaacaaaaagacagtaataaagaaaaagatgataaagcgaaaactgaacaacaagataaaaaaacaaatgattcatctaaagataagaaagacaataaagatgatagtaaagacgtaaacaaagataataaagataatagtgcaaacgataaccagcaacaatctaattcaaatgcaacaaacaatgaccaaaatcaaacgaataataaccagtcaaacagtggacaaacgactaacaatcaaaaatcaagttacgttgcaccatattatggacaaaacgcagcgccagtggctcgtcaaatttatccatttaatggtaataaatcacaagcattacaacaattgcctaatttccaaacagctttaaatgcagctaacaacgaagcaaataaatttggtaatggtcataaagtttataatgattattcaattgaagaacataatggtaactataagtatgtttttagttttaaagacccaaacgtaaatggaaaatattcaattgtaacggttgattatactggacaagcaatggttactgatccaaactaccaacaataa

SEQ ID NO: 36 - RNA, 729 nt, Staphylococcus aureus
  source 1..729: mol_type = mRNA
atgaaaaaagtaatggggatattattagcaagtacacttatcttaggtgcttgtggacatcatcaagatagtgcaaaaaaagagagcactagtcacaaaaagaaagaaaatgacaatgaagaattaaatgaagaacttaaagaatttaaaagcaaaaaaaatatggatataaaaattaaaggcgatactattgttagtgacaaatttgaagctaaaataaaagaaccgtttatcatcaatgaaaaagatgagaaaaagaaatatatcgcttttaaaatggaaattactgctaaaaaagacgataaagatttaaatccatcttctatttctcatgactatattaatatcactcaagatgataaaaatacagtaaataaattaagagatggttatcttttaagtgataaaaattataaagattggacagaacataaccaagatcaaattaaaaaaggcaaaactgcacaagccatgtttatctatgagttaagaggtgatggaaacattaatttaaatgtccataaatactcagaagataaaacagttgattctaaatcattcaaatttagtaaacttaaaaccgaagatttttctcatagagcggaaacaagggaagaagtagaaaagaaagaaaaagaatttgaagaagagtacaaaaaagaacaagaacgagagaaagaaaaagaaaagcaaaaagatgacgaccacagtggtttagatgaagtataa

SEQ ID NO: 37 - RNA, 984 nt, Staphylococcus aureus
  source 1..984: mol_type = mRNA
atgaaaaaatggcaatttgttggtactacagctttaggtgcaacactattattaggtgcttgtggtggcggtaatggtggcagtggtaatagtgatttaaaaggggaagctaaaggggatggctcatcaacagtagcaccaattgtggagaaattaaatgaaaaatgggctcaagatcactcggatgctaaaatctcagcaggacaagctggtacaggtgctggtttccaaaaattcattgcaggagatatcgacttcgctgatgcttctagaccaattaaagatgaagagaagcaaaaattacaagataagaatatcaaatacaaagaattcaaaattgcgcaagatggtgtaacggttgctgtaaataaagaaaatgattttgtagatgaattagacaaacagcaattaaaagcaatttattctggaaaagctaaaacatggaaagatgttaatagtaaatggccagataaaaaaataaatgctgtatcaccaaactcaagtcatggtacttatgacttctttgaaaatgaagtaatgaataaagaagatattaaagcagaaaaaaatgctgatacaaatgctatcgtttcttctgtaacgaaaaacaaagagggaatcggatactttggatataacttctacgtacaaaataaagataaattaaaagaagttaaaatcaaagatgaaaatggtaaagcaacagagcctacgaaaaaaacaattcaagataactcttatgcattaagtagaccattattcatttatgtaaatgaaaaagcattgaaagataataaagtaatgtcagaatttatcaaattcgtcttagaagataaaggtaaagcagctgaagaaggtggatatgtagcagcaccagagaaaacatacaaatcacaattagatgatttaaaagcatttattgataaaaatcaaaaatcagacgacaagaaatctgatgataaaaagtctgaagacaagaaataa

SEQ ID NO: 38 - RNA, 1074 nt, Staphylococcus aureus
  source 1..1074: mol_type = mRNA
atgaaaggtaaatttttaaaagttagttctttattcgttgcaactttgacaacagcgacacttgtgagttctccagcagcaaatgcgttatcttcaaaagctatggacaatcatccacaacaaacgcagacagacaaacagcaaacacctaagattcaaaaaggcggtaaccttaaaccattagaacaacgtgaacgcgctaatgttatattaccaaataacgatcgtcaccaaatcacagatacaacgaatggtcattatgcacctgttacttatattcaagttgaagcacctactggtacatttattgcttctggtgtagttgtaggtaaagatacacttttaacaaataaacacatcgtagatgctacgcacggtgatcctcatgctttaaaagcattcgcttctgcaattaaccaagacaattatcctaatggtggtttcactgctgaacaaatcactaaatattcaggcgaaggtgatttagcaatcgttaaattctcccctaatgagcaaaacaaacatattggcgaagtagttaaaccagcaacaatgagtaataatgctgaaacacaagttaaccaaaatattactgtaacaggatatcctggtgataaacctgtcgcaacaatgtgggaaagtaaaggaaaaataacgtacttaaaaggtgaagcaatgcaatatgatttaagtacaactggtggtaactcaggttcacctgtatttaatgaaaaaaatgaagtcattggcattcattggggtggcgttccaaatcaatttaacggtgcagtatttattaatgaaaatgtacgcaacttcttaaaacaaaatattgaagatatcaatttcgcaaatgatgaccaccctaacaaccctgataatccagacaatccaaataatccggacaatcctaacaaccctgataaccctaacaaccctgataatccagacaatcctaataatcctgataaccctaacaacccggacaatccaaataaccctgaccaacctaacaacccaaataacccggacaatggcgataacaataattcagacaaccctgacgctgcataa

SEQ ID NO: 39 - RNA, 1200 nt, Staphylococcus aureus
  source 1..1200: mol_type = mRNA
atgaagcgtacattagtattattgattacagctatctttatactcgctgcttgtggtaaccataaggatgaccaggctggaaaagataatcaaaaacataacaatagttcaaatcaagtaaaagaaattgctacggataaaaatgtacaaggtgataactatcgtacattgttaccatttaaagaaagccaggcaagaggacttttacaagataacatggcaaatagttataatggcggcgactttgaagatggtttattgaacttaagtaaagaagtgtttccaacagacaaatatttgtatcaagatggtcaatttttggacaagaaaacaattaatgcctatttaaatcttaagtatacaaaacgtgaaatcgataaaatgtctgaaaaagataaaaaagacaagaaagcgaatgaaaatttaggacttaatccatcacacgaaggtgaaacagatcctgaaaagattgcagaaaaatcaccagcctatttatctaacattttagagcaagatttttatggtggtggagatacaaaaggtaagaatattaaaggtatgacgattggtttagctatgaatagtgtttattactataaaaaagaaaaagatggaccgacttttagtaaaaaactagatgatagcgaagttaaaaagcaaggtaaacaaatggctagtgagatattatcaaggttacgtgaaaatgatgatttaaaagatataccaattcattttgcaatttataagcaatcaagtgaagattcaatcacaccaggtgaatttatcactcaagcgactgcagaaaagagtcaaacaaagcttaatgaatggcataatatcaatgaaaaatcagctttattaccttcttcaacagcagcagattatgatgaaaatttaaataataatttcaagcaatttaatgataatttgcaatcatatttttctaatttcacacaagcagtaggaaaagttaaatttgttgataaaaagccacaacgattagtagtagatttaccaatcgattactatggacaagctgaaacaattggtattacacagtacgttactgaacaagcgaataaatatttcgataaaatcgataactatgaaattcggattaaagatggtaaccaaccacgtgctttaattagtaagacaaaagatgacaaagaaccgcaagttcatatttacagtaattaa

SEQ ID NO: 40 - RNA, 1488 nt, Staphylococcus aureus
  source 1..1488: mol_type = mRNA
atgagggaaaattttaagttacgtaaaatgaaagtcgggttagtatctgttgcaattacaatgttatatatcatgacaaacggacaagcagaagcatcagaggctaatgagaagccaagtacaaatcaagaatcaaaagttgtttcacagactgaacaaaattcaaaagaaacaaaaacagtagaatctaataagaactttgttaaattagatactattaaacctggagctcaaaagataacgggaactactttaccaaatcactatgttttattaacagttgatgggaaaagtgcggattcagtagaaaatggcggtttgggttttgttgaagcaaatgacaaaggagaatttgagtaccctttaaataatcgtaaaattgttcataatcaagaaattgaggtttcgtcgtcaagccctgatttaggtgaagatgaagaagatgaagaggtggaagaagcttcaactgataaagctggcgttgaggaagaaagtacagaagctaaagttacttacacaacaccgcgatatgaaaaagcgtatgaaataccgaaagaacaactaaaagaaaaagatggacatcaccaagtttttatcgaacctattactgaaggatcaggtattattaaagggcatacgtctgtaaaaggtaaagttgctttatctattaataataaatttattaattttgaagagagcgttaagggcggagttagtaaagaagacactaaagctagttcagatggtatctggatgcctattgatgacaaaggatactttaactttgacttcaaaacgaaacgtttcgataatttagagttaaaagaaggtaatgacatttcactaacatttgcacctgatgatgaagaagatgcattaaaacctttaattttcaaaactaaagtaacgagcttagaagatatcgataaagcagaaactaaatatgaccatactaaactcaacaaagtgaaagttttagataatgttaaagaagatttacatgttgatgaaatatatggaagcttatatcatacagacaaaggtaaaggtattcttgataaagaaggtactaaagtaattaaaggaaagactaaattcgcgaatgcagtagtgaaggtagactctgaactaggtgaagcacaattattccctgatttacaagtaaatgaaaaaggtgaatttagctttgactcacatggtgctggttttagattacaaaatggagaaaaattaaacttcacagtggttgatcctattacaggtgacttgttaagtaatgagtttgtttctaaagagattgatattgaagaaacacctgaacaaaaagcggatcgtgagtttgacgaaaaacttgaaaatacgcctgcttactacaagttatacggcgataaaatagttggattcgatactaacgatttcccgattacttggttctatccattgggtgaaaagaaagttgaacgtacaacacctaaattagaaaaataa

SEQ ID NO: 41 - RNA, 1524 nt, Staphylococcus aureus
  source 1..1524: mol_type = mRNA
atgtctaaaaagttaaaaattataattcctattattattgtcttattattaataggtggaatcgcatggggagtttatgcattttttgcaaacacaccgaaaaatacatacttaaaaagtgaacaacaaactgcaaaaatgtataaagattattttaatgaccgttttgaaaacgaagtgaagttccaagaaaagatgaaagataattcatttttatcttcattagaattaagcgcagatgcatctgatgaaattgttaaagggcttggtattcctaaatctgttgttaatgcttcgaaaattaaaatgtcatatggacatgatcctaaaaaagagaaatcaatgattaatcttgaaccaacaatagcagactctgcattagggaaattccagttagctgcagataaagataagcattatttcgaatcaccattatttaaagggaaatatagtgttaataattctgatttattatcaacttattcaaaacttacaggtgaagatgaagaaacagcaaaagaaaatggtattacaaaccaacaactaaatttaaatactcttttcagtaatgctcaagcacaacaaagtgactacagcaaaattgccgaaaaatattccgaacttattgtcgacaaattagatgacgataattttgataaaggtaaaaaagaagaaattaaggttaatggtgaaaagtacaaagttagacctgtcacgttaacacttagcagagctgacactaaaaaaattacattagctgtattagaagaagctaaaaaggataaagaccttaaaaaattaatggaagaacaaggtactacaaaagactttgaaaaagacattaaaaaagcaattgacgatgtcaaagaaactaaaaaggatgaatttgctaaaattcaatctaaaatttataccgaaaaacatacgattgtaaaacgagaaattactattacagacaaagaaaataataaaactaaaatcaaaggtactaatactttagaagacgataagttaaaactagattacgcacttgatttcgatcaagataaatacacgtatgctgaagcgaaatatacaattaaaggcgtatcttctaaggaaaaagacaataaatacagtgataaatacgaatttggtaaaaagacagaatatgatgaatcaaaaatcaaattagataaccaagaaaaagtagatggcacaaaacgtcaagataaaggtaaaatcactgtcgcgttagataaatatagcgacgaaaatgaattcacttttgaaaataatatagattctgacgtaaaaaataacactcagaaatctacgttaaatatcggcatcaaatatgctgaagaaccaattaatttcattttaaaatctagcacaaaattgaaagcagatattgattttgatgatagtggtgcgaaagatttcaatagtctatcttcaaaagaccgtgaaaaacttgaaaaagaaatcgaaaaaaatggcggcaaaatgtttgaatcaattttaaaaaaggcatctaaataa

SEQ ID NO: 42 - RNA, 1530 nt, Staphylococcus aureus
  source 1..1530: mol_type = mRNA
gtgaggaaattttcaagatatgcatttacaagtatggcaacagtaacgttgctgagctctttgacacctgcagcactagcgagtgatacgaatcacaaaccagcaacttcagatattaattttgaaatcacgcaaaagagtgatgcagttaaagcattaaaagagttacctaaatctgaaaatgtgaaaaatcattatcaagattactctgttacagatgtaaaaacagataagaaaggattcacgcattacacgttacaaccgagtgtggatggtgtgcatgcgcctgacaaagaagtgaaagtgcatgcggacaaatcgggtaaagtcgttttaatcaacggtgatactgatgcgaagaaagtaaagccgacaaataaagtgacattaagcaaggatgaagcggctgacaaagcatttaacgcagttaagattgataaaaataaagctaaaaacctccaagatgacgttatcaaagaaaataaagtcgaaatcgatggtgacagtaataaatacatttacaatattgaattaattacagtaacaccagaaatttcacattggaaagttaaaattgatgcagacacaggagcagttgttgaaaaaacgaacttagttaaagaagcagcagcaactggcacaggtaaaggtgtgcttggagatacaaaagatatcaatatcaatagtattgatggtggatttagtttagaggatttgacgcatcaaggtaaattatcagcatacaattttaacgatcaaacaggtcaagcgacattaattactaatgaagatgaaaacttcgtcaaagatgatcaacgtgctggtgtagatgcgaattattatgctaaacaaacatatgattactacaaaaatacatttggtcgtgagtcttacgataaccatggtagtccaatagtctcattaacacatgtaaatcattatggtggacaagataacagaaataacgctgcatggattggagacaaaatgatttatggtgatggcgatggccgcacgtttacaaatttatcaggtgcaaatgacgtagtagcacatgagttaacacatggcgtgacacaagaaacggcgaatttagagtataaagatcaatctggtgcgttaaatgaaagcttttcagatgtttttggatactttgtagatgatgaggatttcttgatgggtgaagatgtttacacaccaggaaaagagggagatgctttacgaagcatgtcaaacccagaacaatttggtcaaccatctcatatgaaagactatgtatacactgaaaaagataacggtggtgtgcatacgaattctggcattccaaataaagcagcttataacgtaattcaagcaatagggaaatctaaatcagaacaaatttactaccgagcattaacggaatacttaacaagtaattcaaacttcaaagattgtaaagatgcattataccaagcggctaaagatttatatgacgagcaaacagctgaacaagtatatgaagcatggaacgaagttggcgtcgagtaa

SEQ ID NO: 43 - RNA, 1548 nt, Staphylococcus aureus
  source 1..1548: mol_type = mRNA
atgaaaaagaaattaggtatgttacttcttgtaccagccgtaactttatcattagccgcatgtgggaatgatgatggaaaagataaagatggcaaggtaacaattaaaacgacagtttatccattgcaatcatttgcagagcaaattggtggaaaacacgtgaaggtatcatcaatctatccagcagggacagatttacatagctatgaaccaacacaaaaagatatattaagtgcaagcaagtcagacttgtttatgtatacaggggataatttagatccggttgctaagaaagttgcatctactattaaagataaagataaaaaactgtctttagaagataaattagataaagcaaagcttttaactgatcaacacgaacatggtgaagagcatgaacatgagggacatgatcatgggaaagaagaacatcatcatcatggcggatatgatccacacgtatggttagatcctaaaattaaccaaactttcgctaaagaaattaaagatgaattagtgaagaaagatccaaaacataaagatgactatgagaaaaactacaaaaaattaaacgacgatcttaagaaaattgataacgatatgaagcaagttactaaagataagcaaggtaatgcagtattcatttcacatgaatcaattggatacttagctgatcgttatggttttgttcaaaaaggtattcaaaacatgaatgctgaagatccatcacaaaaagaattgactaaaattgttaaagaaattagagatagcaatgctaaatatattctttacgaagataatgttgcgaataaagtgactgaaacaattcgtaaagaaacagatgcgaagcctttaaaattctacaacatggagtctttaaataaagaacaacagaaaaaagataatattacctatcaatcattaatgaaatcgaatattgaaaatatcggtaaagctttagacagtggtgttaaagtgaaagacgacaaagctgaaagtaaacacgacaaagcaatttctgatgggtattttaaagatgagcaagttaaagaccgtgaattaagcgattatgctggtgaatggcaatctgtttacccttacttaaaagacggtacgcttgatgaagtgatggaacataaagctgaaaatgatccgaagaaatctgctaaagatttaaaagcttattatgacaaaggatataaaactgatattactaacattgatataaaaggaaatgaaattacatttactaaagatggtacgaaacacactggtaaatatgaatacaatggtaagaaaacattgaaatatcctaaaggtaaccgtggcgtgagatttatgtttaaattggtcgatggtaatgataaagacttaccgaaattcatccaatttagcgatcacaacattgcacctaaaaaggcagaacacttccatatctttatgggtaatgataatgacgcgttattaaaagaaatggataactggccaacatattatccttcaaaattaaataaagaccaaatcaaagaagaaatgttagcgcattaa

SEQ ID NO: 44 - RNA, 1695 nt, Staphylococcus aureus
  source 1..1695: mol_type = mRNA
atggtgttatatatcattttggcaataattgtgattatattgattgctgtaggtgtattattctatttacgttcaaataaaagacaaataatagaaaaagcaatcgaacgtaaaaatgaaattgaaacgttaccttttgatcaaaaccttgcacaattatctaagttgaatttaaaaggtgaaacaaaaacgaaatacgatgcaatgaaaaaggacaacgtagaaagtacaaataagtatctagctcctgtggaagaaaaaatccataatgctgaggctttattagataaatttagtttcaacgcatctcaatgtgaaattgatgatgcaaatgagttgatggatagttacgaacaaagctatcagcaacaattagaagatgtaaatgaaattattgcgttatacaaagataatgatgaattatatgacaaatgtaaggttgattatcgtgaaatgaaacgtgatgttttagcaaatcgtcatcaatttggtgaggcagcaagtcttcttgaaactgaaattgaaaaattcgagccaaggttagagcaatatgaagtactaaaagctgatggtaattatgtacaagcgcacaaccatatagctgccttgaatgaacaaatgaaacagctaagatcttatatggaagaaataccagaattaattagagaaactcaaaaagaattacctggtcaattccaagatttaaaatatggttgccgtgatcttaaagttgaagggtatgatctggatcacgtaaaagtagacagtacattacaaagcttaaaaacagagcttagtttcgttgaaccattaattagtcgcttagaattagaagaagctaatgataaactagctaatatcaatgataagttagatgacatgtatgatttaattgaacatgaagttaaagctaaaaatgatgtcgaagaaacaaaagatatcattacggataacttattcaaagcaaaagacatgaattatacattgcaaacagaaattgaatatgtacgtgaaaactactatataaatgaatctgatgctcagagtgttcgtcaatttgaaaatgaaattcaaagtttaatttctgtatatgatgatattttaaaagaaatgtctaaatctgctgtacgatatagcgaggttcaggataatttacaatatttagaagatcatgtcacagttattaatgacaaacaagaaaagctacaaaatcatctgattcaattgcgtgaagatgaagcagaagcagaagacaatctgttacgagtacaatcgaagaaagaagaagtgtatcgtcgattacttgcttctaacttaacaagcgttcctgaaaggtttatcatcatgaaaaatgaaattgatcatgaagttcgtgatgttaacgaacaatttagtgaacgtccaatacacgttaaacagttaaaagataaagtgtctaaaattgtgattcaaatgaatacatttgaagatgaagcaaatgatgttcttgttaatgctgtttatgcagagaaattaattcaatatggaaatagatatcgtaaggactatagcaatgttgataagagcttaaatgaagctgaacgattatttaaaaataatcgctataagcgtgcgattgaaattgcagagcaagctcttgaaagtgttgagccaggtgtcactaaacatattgaagaagaagttattaagcaatag

SEQ ID NO: 45 - RNA, 1860 nt, Staphylococcus aureus
  source 1..1860: mol_type = mRNA
atgcctaaaaataaaattttaatttatttgctatcaactacgctcgtattacctactttagtttcacctaccgcttatgctgacacacctcaaaaagatactacagctaagacaacatctcatgattccaaaaaatctactgatgatgaaacttctaaggatactacaagtaaagatattgataaagcagacaacaataatactagtaaccaagacaataacgacaaaaaagttaaaactatagacgacagcacttcagactctaacaatatcattgattttatttataagaatttaccacaaaccaatataaaccaattgctaaccaaaaataaatacgatgataattactcattaacaactttaatccaaaacttattcaatttaaattcggatatttctgattacgaacaacctcgtaatggtgaaaagtcaacaaatgattcgaataaaaacagtgataatagcatcaaaaatgatacggatacgcaatcatctaaacaagataaagcagacaatcaaaaagcacctaaatcaaacaatacaaaaccaagtacatctaataagcaaccaaattcgccaaagccaacacaaccaaatcaatcaaatagtcaaccagcaagtgacgataaagtaaatcaaaaatcttcatcgaaagataatcaatcaatgtcagattcggctttagattctattttggatcaatacagtgaagatgcaaagaaaacacaaaaagattacgcatctcaatctaaaaaagacaaaaatgaaaaatctaatacaaagaatccacagttaccaacacaagatgaattgaaacataaatctaaacctgctcaatcattcaataacgatgttaatcaaaaggatacacgtgcaacatcactattcgaaacagatcctagtatatctaacaatgatgatagtggacaatttaacgttgttgactcaaaagatacacgtcaatttgtcaaatcaattgctaaagatgcacaccgcattggtcaagataacgatatttatgcgtctgtcatgattgcccaagcaatcttagaatctgactcaggtcgtagtgctttagctaagtcaccaaaccataatttattcggtatcaaaggtgcttttgaagggaattctgttccttttaacacattagaagctgatggtaatcaattgtatagtattaatgctggattccgaaaatatccaagcacgaaagaatcactaaaagattactctgaccttattaaaaatggtattgatggcaatcgaacaatttataaaccaacatggaaatcggaagccgattcttataaagatgcaacatcacacttatctaaaacatatgctacagatccaaactatgctaagaaattaaacagtattattaaacactatcaattaactcagtttgacgatgaacgtatgccagatttagataaatatgaacgttctatcaaggattatgatgattcatcagatgaattcaaacctttccgcgaggtatctgataatatgccatatccacatggccaatgtacttggtacgtatataaccgtatgaaacaatttggtacatctatctcaggtgatttaggtgatgcacataattggaataatcgagctcaataccgtgattatcaagtaagtcatacaccaaaacgtcatgctgctgttgtatttgaggctggacaatttggtgcagatcaacattacggtcatgtagcatttgttgaaaaagttaacagtgatggttctatcgttatttcagaatccaatgttaaaggattaggtatcatttctcatagaactatcaatgcagctgccgctgaagaattatcatatattacaggtaaataa

SEQ ID NO: 46 - RNA, 2046 nt, Staphylococcus aureus
  source 1..2046: mol_type = mRNA
atgatgaaaagtcaaaataagtatagtattcgtaaatttagtgtaggtgcatcttccattttaatagctacattactatttttaagtggtggacaagcacaagcagctgagaagcaagtgaatatgggaaattcacaggaggatacagttacagcacaatctattggggatcaacaaactagggaaaatgctaattatcaacgtgaaaacggtgttgacgaacagcaacatactgaaaatttaactaagaacttgcataatgataaaacaatatcagaagaaaatcatcgtaaaacagatgatttgaataaagatcaactaaaggatgataaaaaatcatcgcttaataataaaaatattcaacgtgatacaacaaaaaataacaatgctaatcctagggatgtaaatcaagggttagaacaggctattaatgatggcaaacaaagtaaagtggcgtcacagcaacagtcaaaagaggcagataatagtcaagacttaaacgctaataacaatctaccttcacaaagtcgaacaaaggtatcaccatcattaaataagtcagatcaaacaagtcaacgagaaattgttaatgagacagaaatagagaaagtacaaccgcaacaaaagaatcaagcgaatgataaaattactgaccacaattttaacaatgaacaagaagtgaaacctcaaaaagacgaaaaaacactatcagtttcagatttaaaaaacaatcaaaaatcaccagttgaaccaacaaaggacaatgacaagaaaaatggattaaatttattaaaaagtagtgcagtagcaacgttaccaaacaaagggacaaaggaacttactgcaaaagcgaaaggtgatcaaacgaataaagttgccaaacaagggcagtataaaaatcaagatcctatagttttagtgcatggtttcaatgggtttacagatgatattaatccttcagtgttagctcattattggggcggtaataaaatgaacattcgccaagatttagaagaaaatggttacaaagcttatgaagcaagtataagtgcttttggaagtaactatgaccgcgcagttgaactttattattatatcaaaggcggtcgtgtagattatggtgcagcacatgcagcaaaatatggacatgaacgttatggaaaaacatacgaaggaatttacaaagactggaaaccaggacagaaggtacaccttgttggacatagtatgggtggtcaaacgatacgtcaactagaagaattactgcgtaatggtagtcgtgaagaaatagagtatcaaaagaaacatagtggcgaaatttctccactattcaaaggtaataatgacaatatgatttcatcaattactactttaggaacgccacataatggaacgcatgcttcagatttagctggtaatgaagctttagtgagacaaattgtatttgatatcggtaaaatgtttggtaataaaaattcaagagtagacttcgggttggctcaatggggtctaaaacagaagccaaatgaatcatatattgattatgtcaaacgcgttaaacaatctaatttatggaaatcaaaagataatggattttacgatctgacgcgtgagggtgcaacagatttaaatcgtaaaacgtcgttgaaccctaacattgtgtataaaacatacactggtgaagcaacgcacaaagcattaaatagcgatagacaaaaagcagacttaaatatgtttttcccatttgtgattactggtaacttaatcggtaaagctactgaaaaagaatggcgagaaaacgatggtttagtatccgttatttcttctcaacatccatttaatcaagcttatacaaatgcgacggataaaattcaaaaaggcatttggcaagtaacgcctacaaaacatgattgggatcatgttgattttgtcggacaagatagttctgatacagtgcgcacaagagaagaattacaagatttttggcatcatttagcagacgatttagtgaaaactgaaaaggtgactgatactaagcaagcataa

SEQ ID NO: 47 - RNA, 2421 nt, Staphylococcus aureus
  source 1..2421: mol_type = mRNA
atgacaaataaaatgaagaaatggcaaaaattatccaccattacgttattaatgaccggagtgattgctttaaataatggtgaatttagaaatgttgataaacatcaaatcgctgtggctgatacgaatgttcaaacgccagattatgaaaaattgaagaagacgtggctcgacgttaactacggttatgatcagtatgatgagaataatcaagatatgaagaagaagtttgatgctaaagaaaaagaagccaagaagttacttgatgacatgaaaactgatacgaatagaacatatttgtggtcaggagctgaaaaccttgaaactaattcttctcacatgacaaaaacctatcgtaatatcgagaaaatcgcagaatcaatgcaacataagaatacggtattaaaaacagttgaaaacaagttgaaaataaaagaagccctagattggatgcacaaaaatgtttatggcaagaatccttctcaaaaagtcgaggatttaactaaaaatcgtaaggggcaaactacacccaagaataactcattgaattggtgggattatgaaattggtacgccaagagcattaacaaatacactacttctaatggatgatatgctcactaaagatgaaatgaaaaattattcaaaacctattagtacatatgcaccatccagtgacaaaattttatcttctgttggtgaatcagaagatgctaaaggtggaaatttagtggacatttctaaagtaaaacttttagaaagtgttattgaagaagatgtagatatgttgaaaaagtctatagattcttttaataaagtgttcacttatgttcaagattctgccactggtaaaggtcgcaatggattctataaagatggctcttacattgatcatcaagatgtcccttacactggtgcttatggtgttgtactattagagggtatttctcaaatgatgccgatgataaaagaatctccttttaaaactacacaagataatgctacattaagcaattggattgacgaagggtttatgccattaatctataaaggtgaaatgatggatttatcacgaggtagagctatcagtcgtgaaaatgaaacgagtcatacagcgtcagcgactgtaatgaaatcattgttgagattgaatgataccatggatgattcaacaaaaactagatataagcaaatcgttaaaacttctgttaattctgattcaagttacaaccaaaataattatttaaattcatattcagacatagctaaaatgaaaaagttaatgaatgatagtactatttctaaaaacgatttaacacagcaacttaaaatatataatgacatggatcgtgtcacctatcacaataaagacctggactttgcatttggtttaagtatgacatcgaaaaacatcgcacgatacgaaaatatcaacggagagaacttaaaaggttggcacaccggtgcaggcatgtcttatttatataacagcgatgtcaaacactatcgcgataacttctgggcaacagccgatatgacttgtcttccaggcactactactttaaatgatatgccatctactaatactaagaatgataaatcttttgttggcgggacaaaattaaataataaatacgcaagcatcggtatggattttgaaaatcaggacaaaactttaactgccaaaaaatcatatttcatattaaacgataaaattgtcttcttaggaactggcattaaaagtactgattcatcaaagaatccagttacaagtgttgaaaatcgcaaagcaaatgggtataaattatttaaagatgatattgaaattaccacttcagatgttaatgctcaggaaacccattcagtctttttagagtccaacgatactaaaaagaacattggttatcatttcttagacaagccaaaaataactgtaaaaaaagaaagtcatactggtaagtggagtgaaattaataaaagtcaaaaaaaagatgacaaaaaagatgagtattatgaagtaactcaaacacataatacatctgacagtaaatatgcatatgttttgtatcctggtttatcaaaaagtgattttaaatcgaagaataataatgtaagtattgttaaacaagatgaagattttcatgtgataaaagataatgatggcgtatttgctggggttaattatagtgataatactaaatcttttgatataaacggaattactgttgaattaaaagaaaaaggcatgtttgtaattaaaaagaaagatgataaagcatataaatgtagcttctataatcctgaaactacaaataccgcttcaaatatagaatcaaaaatttttattaaaggttacaccataactaataaaagtgtcataaactctaatgatgctggtgtaaactttgaattaactaaataa

SEQ ID NO: 48 - RNA, 2427 nt, Staphylococcus aureus
  source 1..2427: mol_type = mRNA
atgacatatagaatgaagaaatggcaaaaattgtccaccattacgttattaatggctggtgtgattactttgaatggtggtgaattcagaagtattgataaacatcaaatcgctgtggctgatacgaatgttcaaacgacagattatgaaaagttgaggaacatatggctggacgttaactatggttatgataagtatgatgagaataatccagatatgaagaagaagtttgaggctacggagaatgaggcagagaaattactcaaggaaatgaaaactgaaagtgataggaaatacttgtgggaaagctcaaaagatttagatacgaagtctgcggatatgactcgtacctatcgtaatattgagaaaatctcagaagcgatgaaacataaaaatactaaattaaaaacagatgaaaacaagacaaaagtaaaagatgcacttgagtggctgcataaaaatgcatatggaaaagaaccagataaaaaagttgctgatttgacctcaaactttaaaaataaaacttctagaaataccaacttaaattggtgggattatgaaattggaacacctagagcattaacaaatacgcttatactcttacaagaagatttcactgatgaagaaaagaaaaaatatacagctcctattaaaactttcgccccagatagtgacaaaatattatcttctgtaggaaaatctgaacctgctaaaggcggaaatttagtagacatttctaaagtaaaacttttagaaagtattatcgaagaagacaaagatatgatgaaaaagtctatagattcatttaatacagtcttcacttacgcgcaaaattctgccactggaaaagaacgtaatggattctataaagatggctcttacattgatcatcaagacgtcccatacactggtgcttatggcgttgtactattagagggtatttctcaaatgatgccgatgataaaagaaacaccttttaatgatagtaaccaaaatgatacaaccttaaaatcatggattgacgacggatttatgccactcatttataaaggtgaaatgatggatttatcaagaggtagagctatcagtcgtgaaaatgaaacgagtcactcagcatctgcaacagtaatgaaatcattgttgagattgagtgataccatggataagtctacaaaagctaaatataaaaagattgtcaagacttcagtagagtcagattcaagttataaacaaaccgattatttaagctcttattcggatataagcaaaatgaagtctttaatggaagacagcactatttctactaacggtttaacacaacaacttaaaatatataatgacatggatcgtgtcacctatcacaataaaggcttagactttgcatttggtttaagtatgacgtcgaaaaacgtcgcacgttacgaaagtatcaacggagagaacttaaaaggttggcacactggtgctggaatgtcttatttatacaatagcgatgtgaaacactaccgtgataacttctgggcgacagctgatatgaaacgtttagcaggtactacaactttagataatgaagaacctaaaagtacggatgttaaaaagtctagtaaaacttttgtaggaggaacaaaattcgatgaccaacatgctagtatcggaatggattttgaaaatcaggacaaaactttaactgccaaaaaatcatatttcatattaaacgataaaattgtcttcttaggaactggcattaaaagtactgattcatcaaagaatccagttacaacgattgaaaatcgcaaagcgaatgattataaattatataaagatgatacgcaaacaaccaattccgataatcaggaaaccaattccctctttttagagtcaacgaatagcactcaaaacaatataggttatcattttttaaacgaatcgaaaataactgtaaaaaaagaaagtcatactggtaagtggagtgatataaataaaagccaaaaggatatacaaaaaactgatgagtattatgaagtaactcaaaagcattctaatacagatagtaaatatgcatatgtgttgtatccaggcttatctaaagatgtctttaaatccaaagcaagcaaagtaactgtcgttaagcaagaagatgacttccacgttgtgaaagataatgaatcggtttgggctggtatcaattatagtgatagcgctaaaacttttgaaattaataacactaaagtcgaagttaaagccaaaggaatgtttattcttacaaagaaagatgataacacttatgaatgtagcttctataatcccgaatctacaaattccgtttcagatattgaatctaaaatttcaatgactggatactctattataaacaaaaatacgtcgacttctaatgaatccggcgtacgctttgaattaactaaataa

SEQ ID NO: 49 - protein, 412 aa, Staphylococcus aureus
IDSKNKPANSDIKFEVTQKSDAVKALKELPKSENVKNIYQDYAVTDVKTDKKGFTHYTLQPSVDGVHAPDKEVKVHADKSGKVVLINGDTDAKKVKPTNKVTLSKDDAADKAFKAVKIDKNKAKNLKDKVIKENKVEIDGDSNKYVYNVELITVTPEISHWKVKIDAQTGEILEKMNLVKEAAETGKGKGVLGDTKDININSIDGGFSLEDLTHQGKLSAFSFNDQTGQATLITNEDENFVKDEQRAGVDANYYAKQTYDYYKDTFGRESYDNQGSPIVSLTHVNNYGGQDNRNNAAWIGDKMIYGDGDGRTFTSLSGANDVVAHELTHGVTQETANLEYKDQSGALNESFSDVFGYFVDDEDFLMGEDVYTPGKEGDALRSMSNPEQFGQPAHMKDYVFTEKDNGGVHTNS

SEQ ID NO: 50 - DNA, 1239 bp, Staphylococcus aureus
  source 1..1239: mol_type = genomic DNA
attgattcaaaaaataaaccagctaattctgatattaaatttgaggtgactcaaaagagtgatgcggtcaaagcattaaaagaattgcctaaatccgaaaatgtaaaaaatatttatcaagattacgctgttactgatgtaaaaactgataaaaaaggatttacgcattatacattgcaaccgagtgttgatggtgttcatgcacctgacaaagaagtgaaagtacacgcagacaaatcaggaaaagtcgttttaatcaatggggatactgatgcgaagaaagtaaagccaacgaataaagtgacattaagtaaagatgacgcagccgacaaagcatttaaagcagttaagattgataagaataaagcgaaaaatcttaaagataaagtcattaaagaaaacaaagttgaaatcgatggtgacagtaataaatacgtttataatgttgagttaattacagtgacaccagaaatttcacattggaaagttaaaattgatgctcaaactggcgaaattttagaaaaaatgaacttagttaaagaagctgcagaaactggtaaaggaaaaggtgtacttggcgatacaaaagatatcaatatcaatagtattgacggtggatttagcctagaagatttaacgcatcaaggtaaattatcagcatttagctttaatgatcaaacaggtcaagcaacattgattactaatgaagatgaaaacttcgtaaaagatgagcaacgtgctggcgtagatgcaaattattacgctaaacaaacatatgattattacaaagacacatttggtcgtgaatcatatgacaaccaaggtagtccaattgtttcattaacgcatgttaataactacggtggtcaagataacagaaataatgccgcatggatcggtgacaaaatgatctatggtgatggtgatggtcgcacattcacaagtttatcgggtgcaaatgacgtagtagcacacgaattaacacacggtgtgacacaagagacagcgaacttagaatataaggaccagtcaggcgctctaaatgaaagcttttcagatgtttttggatactttgtagatgacgaggatttcttaatgggtgaagatgtctacacacctggaaaagagggagacgctttacgcagcatgtcaaacccagaacaatttggtcaaccagctcatatgaaagactatgtattcactgaaaaagataatggtggcgtacatacgaattcttaa

SEQ ID NO: 51 - RNA, 1239 nt, Staphylococcus aureus
  source 1..1239: mol_type = mRNA
attgattcaaaaaataaaccagctaattctgatattaaatttgaggtgactcaaaagagtgatgcggtcaaagcattaaaagaattgcctaaatccgaaaatgtaaaaaatatttatcaagattacgctgttactgatgtaaaaactgataaaaaaggatttacgcattatacattgcaaccgagtgttgatggtgttcatgcacctgacaaagaagtgaaagtacacgcagacaaatcaggaaaagtcgttttaatcaatggggatactgatgcgaagaaagtaaagccaacgaataaagtgacattaagtaaagatgacgcagccgacaaagcatttaaagcagttaagattgataagaataaagcgaaaaatcttaaagataaagtcattaaagaaaacaaagttgaaatcgatggtgacagtaataaatacgtttataatgttgagttaattacagtgacaccagaaatttcacattggaaagttaaaattgatgctcaaactggcgaaattttagaaaaaatgaacttagttaaagaagctgcagaaactggtaaaggaaaaggtgtacttggcgatacaaaagatatcaatatcaatagtattgacggtggatttagcctagaagatttaacgcatcaaggtaaattatcagcatttagctttaatgatcaaacaggtcaagcaacattgattactaatgaagatgaaaacttcgtaaaagatgagcaacgtgctggcgtagatgcaaattattacgctaaacaaacatatgattattacaaagacacatttggtcgtgaatcatatgacaaccaaggtagtccaattgtttcattaacgcatgttaataactacggtggtcaagataacagaaataatgccgcatggatcggtgacaaaatgatctatggtgatggtgatggtcgcacattcacaagtttatcgggtgcaaatgacgtagtagcacacgaattaacacacggtgtgacacaagagacagcgaacttagaatataaggaccagtcaggcgctctaaatgaaagcttttcagatgtttttggatactttgtagatgacgaggatttcttaatgggtgaagatgtctacacacctggaaaagagggagacgctttacgcagcatgtcaaacccagaacaatttggtcaaccagctcatatgaaagactatgtattcactgaaaaagataatggtggcgtacatacgaattcttaa

SEQ ID NO: 52 - DNA, 10 bp, synthetic construct
  source 1..10: mol_type = other DNA
attcggaacc

SEQ ID NO: 53 - DNA, 10 bp, synthetic construct
  source 1..10: mol_type = other DNA
atacgggacc